SEQUENCE LISTING (33 sequences)

SEQ ID NO: 1 - RNA, 19 nt, Artificial Sequence
  Oligomeric Compound
aaguaaggaccagagacaa

SEQ ID NO: 2 - RNA, 19 nt, Artificial Sequence
  Oligomeric Compound
uugucucugguccuuacuu

SEQ ID NO: 3 - DNA, 18 bp, Artificial Sequence
  Oligomeric Compound
tgtctctggtccttactt

SEQ ID NO: 4 - RNA, 20 nt, Artificial Sequence
  Oligomeric Compound
aaguaaggaccagagacaaa

SEQ ID NO: 5 - RNA, 20 nt, Artificial Sequence
  Oligomeric Compound
uucauuccuggucucuguua

SEQ ID NO: 6 - RNA, 19 nt, Artificial Sequence
  Oligomeric Compound
ggagaucaacauuuucaaa

SEQ ID NO: 7 - DNA, 20 bp, Artificial Sequence
  Oligomeric Compound
tccgtcatcgctcctcaggg

SEQ ID NO: 8 - DNA, 20 bp, Artificial Sequence
  Oligomeric Compound
gtgcgcgcgagcccgaaatc

SEQ ID NO: 9 - DNA, 20 bp, Artificial Sequence
  Oligomeric Compound
atgcattctgcccccaagga

SEQ ID NO: 10 - protein, 16 aa, Artificial Sequence
  Delivery peptide
RQIKIWFQNRRMKWKK

SEQ ID NO: 11 - protein, 13 aa, Artificial Sequence
  Delivery peptide
GRKKRRQRRRPPQ

SEQ ID NO: 12 - protein, 27 aa, Artificial Sequence
  Delivery peptide
GWTLNSAGYLLGPINLKALAALAKKIL

SEQ ID NO: 13 - protein, 34 aa, Artificial Sequence
  Delivery peptide
DAATATRGRSAASRPTERPRAPARSASRPRRPVE

SEQ ID NO: 14 - protein, 18 aa, Artificial Sequence
  Delivery peptide
KLALKLALKALKAALKLA

SEQ ID NO: 15 - protein, 27 aa, Artificial Sequence
  Delivery peptide
GALFLGWLGAAGSTMGAWSQPKKKRKV

SEQ ID NO: 16 - protein, 16 aa, Artificial Sequence
  Delivery peptide
AAVALLPAVLLALLAP

SEQ ID NO: 17 - protein, 7 aa, Artificial Sequence
  Delivery peptide
PKKKRKV

SEQ ID NO: 18 - protein, 4 aa, Artificial Sequence
  Delivery peptide
MLFY

SEQ ID NO: 19 - protein, 15 aa, Artificial Sequence
  Delivery peptide
PQRRNRSRRRRFRGQ

SEQ ID NO: 20 - protein, 7 aa, Artificial Sequence
  Delivery peptide
IMRRRGL

SEQ ID NO: 21 - protein, 11 aa, Artificial Sequence
  Delivery peptide
LQLPPLERLTL

SEQ ID NO: 22 - protein, 11 aa, Artificial Sequence
  Delivery peptide
ELALKLAGLDI

SEQ ID NO: 23 - protein, 11 aa, Artificial Sequence
  Delivery peptide
DLQKKLEELEL

SEQ ID NO: 24 - protein, 12 aa, Artificial Sequence
  Delivery peptide
ALPHAIMRLDLA

SEQ ID NO: 25 - protein, 7 aa, Artificial Sequence
  Delivery peptide
PKKKRKV

SEQ ID NO: 26 - protein, 13 aa, Artificial Sequence
  Delivery peptide
ALWKTLLKKVLKA

SEQ ID NO: 27 - protein, 8 aa, Artificial Sequence
  Delivery peptide
FCFWKTCT

SEQ ID NO: 28 - protein, 9 aa, Artificial Sequence
  Delivery peptide
CGNKRTRGC

SEQ ID NO: 29 - protein, 9 aa, Artificial Sequence
  Delivery peptide
GHKAKGPRK

SEQ ID NO: 30 - protein, 4 aa, Artificial Sequence
  Delivery peptide
KDEL

SEQ ID NO: 31 - DNA, 26 bp, Artificial Sequence
  PCR Primer
aatggctaagtgaagatgacaatcat

SEQ ID NO: 32 - DNA, 25 bp, Artificial Sequence
  PCR Primer
tgcacatatcattacaccagttcgt

SEQ ID NO: 33 - DNA, 30 bp, Artificial Sequence
  PCR Primer
ttgcagcaattcactgtaaagctggaaagg

(19) United States
(12) Patent Application Publication
(10) Pub. No.: US 2009/0203132 A1
(43) Pub. Date: Aug. 13, 2009

(54) PYRROLIDINYL GROUPS FOR ATTACHING CONJUGATES TO OLIGOMERIC COMPOUNDS

(76) Inventors: Eric E. Swayze, Carlsbad, CA (US); Dale E. Robinson, JR., San Marcos, CA (US); Elizabeth Anne Jefferson, La Jolla, CA (US); Prasad Dande, Chicago, IL (US); Thazha P. Prakash, Carlsbad, CA (US); Charles Allerson, San Diego, CA (US); Balkrishen Bhat, Carlsbad, CA (US)

Correspondence Address: WOODCOCK WASHBURN LLP, CIRA CENTRE, 12TH FLOOR, 2929 ARCH STREET, PHILADELPHIA, PA, 19104-2891, US

(21) Appl. No.: 11/574,396

(22) PCT Filed: Sep. 1, 2005
(86) PCT No.: PCT/US05/31269

§ 371 Date: Dec. 10, 2007

Related U.S. Application Data
(60) Provisional application No. 60/608,201, filed on Sep. 9, 2004.

(51) Int. Cl. C12N 5/06; C07H 21/02

(52) U.S. Cl. 435/375; 536/22.1; 435/325

(57) ABSTRACT

The present invention provides pyrrolidinyl compounds that are useful for preparing conjugated oligomeric compounds. The conjugated pyrrolidinyl compounds can be attached to support medium and provide a free hydroxyl for oligomer synthesis to prepare an oligmeric compound having a 3′-conjugate. Alternatively, the pyrrolidinyl compound can be prepared as a phosphoramidite which can be placed internally or at the 5′-position of an oligomeric compound. These two strategies can be used together to prepare oligomeric compounds having 2 or more conjugates at any selected positions. The present invention also provides methods for modulating gene expression using the conjugated oligomeric compounds.

### CROSS-REFERENCE TO RELATED APPLICATIONS

This application claims priority to U.S. Provisional Application No. 60/608,201 filed Sep. 9, 2004, which is incorporated herein by reference in its entirety.

### FIELD OF THE INVENTION

The present invention relates to compounds useful for attaching conjugates to oligomeric compounds, methods of their synthesis and applications thereof. More particularly the invention provides novel pyrrolidinyl groups that are useful for attaching conjugate groups to oligomeric compounds. In one aspect of the invention pyrrolidinyl groups attach conjugate groups to support medium and provide further free hydroxyl groups for oligomer synthesis. In a further aspect of the invention conjugated pyrrolidinyl groups are prepared having an activated phosphite moiety for incorporation at any selected position(s) during the synthesis of oligomeric compounds.

### BACKGROUND OF THE INVENTION

Research to discover reagent, diagnostic and therapeutic applications for antisense oligomeric compounds continues to grow with initial focus on single stranded compounds now expanded to single and double stranded compositions. As potential therapeutics, many modifications have been made to native RNA and DNA compositions in an attempt to improve desired properties such as stability to nucleases, specificity and potency. One modification that can impart a number of desired properties to oligomeric compounds is conjugation. Conjugates have been used to improve many properties including cellular targeting and cellular uptake. The ability to target a specific group of cells within a tissue reduces the toxicity to normal cells and increases the activity of an oligoeric compound within the targeted cells.

Oligomeric compounds can be conjugated to groups that bind cell surface receptors thereby resulting in receptor mediated endocytosis. A further useful family of conjugates includes lipids which are able to penetrate cellular membranes and can facilitate cellular uptake of oligomeric compounds. The targeting of cell surface receptors is an example of an active transport mechanism of cellular uptake while the use of lipids to facilitate uptake is a passive mechanism. Many other classes of conjugates have been examined for improving properties of oligomeric compounds such as but not limited to vitamins, peptides, fatty acid side chains, hormones and carbohydrates.

A large genus of molecules including pyrrolidinyl groups are disclosed in published U.S. application US 2005/0107325 for attachment of conjugates to oligomeric compounds however the orientation of incorporation is different from the present invention.

A genus of heterocyclic amines used to attach a limited number of conjugate groups to oligomeric compounds are disclosed in published PCT Appplication WO 03/104249 A1. The pyrrolidinyl group is exemplified as one of the heterocyclic amines however the orientation of incorporation is different from the present invention.

### SUMMARY OF THE INVENTION

The present invention provides of the formula:

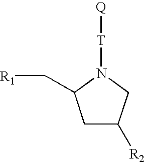

wherein:

R₁ is hydroxyl, a protected hydroxyl, an activated phosphite group, X₁-Y or J-SM;
J is a bivalent linking moiety;
SM is a support medium;

R₂ is hydroxyl, a protected hydroxyl or X₂-Y;
X₁ is an internucleoside linking group connecting a 5′-position of a nucleoside, nucleotide, an oligonucleoside, oligonucleotide or an oligomeric compound;
X₁ is an internucleoside linking group connecting a 3′-position of a nucleoside, nucleotide, an oligonucleoside, oligonucleotide or an oligomeric compound;
Y is a nucleoside, nucleotide, an oligonucleoside, oligonucleotide or an oligomeric compound;

T is a bivalent tethering moiety; and

Q is a conjugate group.

In one embodiment R₁ is hydroxyl or a protected hydroxyl wherein preferred protecting groups trityl, monomethoxytrityl, dimethoxytrityl, trimethoxytrityl, 9-phenylxanthin-9-yl (Pixyl) or 9-(p-methoxyphenyl)xanthin-9-yl (MOX). In a further embodiment R₁ is J-SM where a preferred J-SM has the formula:

C(═O)—R₄—C(═O)—SM

wherein
R₄ is C₁-C₁₂ alkyl or substituted C₁-C₁₂ alkyl, wherein said alkyl group can be interrupted by one or more heteroatoms selected from N(Rₐ), S and O;
   Rₐ is H, C₁-C₁₂ alkyl or substituted C₁-C₁₂ alkyl; and
SM is a support medium. In a preferred embodiment R₄ is C₁-C₁₂ alkyl with a more preferred alkyl being CH₂CH₂.

In one embodiment R₁ is X₁-Y where a preferred X₁ is phosphodiester, phosphorothioate or chiral phosphorothioate. In another embodiment Y is an oligomeric compound.

In one embodiment the SM is aminoalkyl controlled pore glass (CPG).

In one embodiment R₂ is hydroxyl or a protected hydroxyl. In another embodiment R₂ is X₂-Y wherein a preferred X₂ is a phosphodiester, phosphorothioate or a chiral phosphorothioate. In another embodiment X₂ is a phosphodiester, phosphorothioate or a chiral phosphorothioate and Y is an oligomeric compound.

In one embodiment the bivalent tethering moiety has the formula:

*-C(═O)-E-N(Rₐ)—

wherein

* is attached to the N atom of the pyrrolidinyl group; and

E is a C₁-C₁₂ alkyl or substituted C₁-C₁₂ alkyl, wherein said alkyl groups are optionally further interrupted with from 1 to 5 heteroatoms selected from O, S or N(Rₐ), said substituent groups are selected from ═O and N(Rₐ).

Rₐ is H or C₁-C₁₂ alkyl. Wherein a preferred bivalent tethering moiety is selected from —C(═O)—CH₂—O—(CH₂)₂—O—(CH₂)₂—N(Rₐ)— and —C(═O)—(CH₂)₅—N(Rₐ)—.

In one embodiment the activated phosphite moiety comprises a phosphoramidite, H-phosphonate, phosphate triester or a chiral auxiliary.

In one embodiment at least one Y group is an oligomeric compound.

The present invention also provides for compounds having formula II:

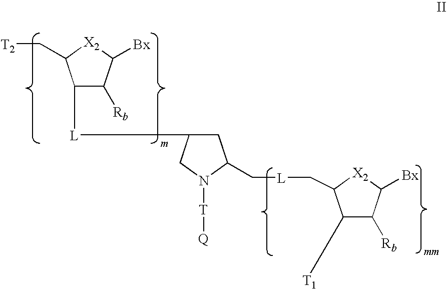

wherein:

T₁ and T₂ are each independently, hydroxyl, a protected hydroxyl or a linkage to a conjugate group;

each L is an internucleoside linking group;

each X₂ is independently, O or S;

each Bₓ is a heterocyclic base moiety;

each R_{b} is independently, H, OH or a 2′-sugar substituent group;

T is a bivalent tethering moiety; and

Q is a conjugate group

m is 0 or from 1 to about 80;

mm is 0 or from 1 to about 80 and

wherein the sum of m plus mm is from 1 to about 80.

In one embodiment m is 0 and in another embodiment mm is 0. In a further embodiment m is at least 1 and mm is at least 1.

In one embodiment each L is independently, a phosphodiester or phosphorothioate internucleoside linking group.

In one embodiment the compound of formula II comprises a first oligomeric compound and further comprising a second oligomeric compound wherein:

at least a portion of said first oligomeric compound is capable of hybridizing with at least a portion of said second oligomeric compound;

at least a portion of one of said first and said second oligomeric compounds is complementary to and capable of hybridizing to a selected nucleic acid target; and

said first and said second oligomeric compounds optionally further comprise one or more overhangs, phosphate moieties or capping groups.

In one embodiment the first and the second oligomeric compounds comprise a siRNA duplex.

In one embodiment Q is a lipophilic moiety, vitamin, polymer, peptide, protein, nucleic acid, small molecule, oligosaccharide, carbohydrate cluster, intercalator, minor groove binder, cleaving agent, or cross-linking agent.

In one embodiment Q is a steroid. In another embodiment Q is cholesterol or a cholesterol derivative. In a further embodiment Q binds to low-density lipoprotein. In even a further embodiment Q is folate or folate derivative. In another embodiment Q is a water-soluble polymer. In a further embodiment Q comprises polyethylene glycol or copolymer thereof where a preferred polyentylene glycol or copolymer thereof has a molecular weight of about 20,000 daltons.

In one embodiment Q comprises a fusogenic peptide or delivery peptide. In a further embodiment Q comprises a drug. In another embodiment Q binds to human serum albumin. In a further embodiment Q comprises a reporter group.

The present invention also provides methods of using the compounds and compositions in therapy.

### DETAILED DESCRIPTION OF THE INVENTION

The present invention provides pyrrolidinyl groups useful for attaching conjugate groups to oligomeric compounds. More particularly pyrrolidinyl groups are orthogonally protected and functionalized with conjugate groups. Deprotection provides a site for attachment of a support medium. Further deprotection provides a free hydroxyl group ready for oligomerization. Alternatively, the pyrrolidinyl can be prepared as a monomer for oligomer synthesis, such as a conjugated phosphoramidite. The orthogonal protection scheme provides for performing the steps in any desired order and provide for the placement of the conjugated pyrrolidinyl group at either the 5′ or 3′-terminus or at any internal position. The method also provides for putting identical or different conjugate groups at multiple selected sites within an oligomeric compound. The conjugated pyrrolidinyl groups are also amenable to solution phase synthesis. The oligomerization process can utilize any number of different monomer synthons to synthesize oligomeric compounds that have a wide variety of chemical modifications which define a variety of motifs. Cleavage from a support medium or purification from a reaction mixture provides the conjugated oligomeric compound.

In one aspect of the present invention conjugated oligomeric compounds are useful for the modulation of gene expression. The conjugated oligomeric compounds can be used as a single stranded compound or can be used as a double stranded composition. In one aspect of the present invention a targeted cell, group of cells, a tissue or an animal is contacted with a compound or composition of the invention to effect reduction of message that can directly inhibit gene expression. In another aspect, the reduction of message indirectly upregulates a non-targeted gene through a pathway that relates the targeted gene to the non-targeted gene. Methods and models for the regulation of genes using oligomeric compounds of the invention are illustrated in the examples.

In another aspect a method of inhibiting gene expression is disclosed comprising contacting one or more cells, a tissue or an animal with a conjugated oligomeric compound of the invention. Numerous procedures of how to use the compositions of the present invention are illustrated herein.

The pyrrolidinyl group has the formula:

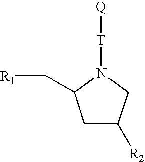

wherein:

R₁ is hydroxyl, a protected hydroxyl, an activated phosphite group or J-SM;
J is a bivalent linking moiety;
SM is a support medium;

R₂ is hydroxyl, a protected hydroxyl or X-Y;
X is an internucleoside linking group;
Y is a nucleoside, nucleotide, an oligonucleoside, oligonucleotide or an oligomeric compound;

T is a bivalent tethering moiety; and

Q is a conjugate group.

The present invention is amenable to all manner of conjugate groups including but not limited to those known in the art. Conjugate groups are attached to oligomeric compounds to enhance desired properties or for tracking of the oligomeric compound or its metabolites. Properties that are typically enhanced include without limitation activity, cellular distribution and cellular uptake. Some representative conjugate groups amenable to the present invention include but are not limited to intercalators, reporter molecules, polyamines, polyamides, polyethylene glycols, polyethers, cholesterols, lipids, phospholipids, biotin, phenazine, folate, phenanthridine, anthraquinone, acridine, fluoresceins, rhodamines, coumarins, dyes, groups that enhance the pharmacodynamic properties of oligomers and groups that enhance the pharmacokinetic properties of oligomers. Groups that enhance the pharmacodynamic properties, in the context of this invention, include groups that improve properties including but not limited to cellular uptake, resistance to degradation and hybridization with RNA. Groups that enhance the pharmacokinetic properties, in the context of this invention, include groups that improve properties including but not limited to oligomer uptake, distribution, metabolism and excretion.

The present invention is amenable to all manner of conjugate groups including but not limited to those known in the art. In one aspect of the invention conjugate groups are attached to oligomeric compounds to enhance desired properties or for tracking of the oligomeric compound or its metabolites. Properties that are typically enhanced include without limitation activity, cellular distribution and cellular uptake. Some representative conjugate groups amenable to the present invention include but are not limited to intercalators, reporter molecules, polyamines, polyamides, polyethylene glycols, polyethers, cholesterols, lipids, phospholipids, biotin, phenazine, folate, phenanthridine, anthraquinone, acridine, fluoresceins, rhodamines, coumarins, dyes, groups that enhance the pharmacodynamic properties of oligomers and groups that enhance the pharmacokinetic properties of oligomers. Groups that enhance the pharmacodynamic properties, in the context of this invention, include groups that improve properties including but not limited to cellular uptake, resistance to degradation and hybridization with RNA. Groups that enhance the pharmacokinetic properties, in the context of this invention, include groups that improve properties including but not limited to oligomer uptake, distribution, metabolism and excretion.

Further representative conjugate moieties can include lipophilic molecules (aromatic and non-aromatic) including steroid molecules; proteins (e.g., antibodies, enzymes, serum proteins); peptides; vitamins (water-soluble or lipid-soluble); polymers (water-soluble or lipid-soluble); small molecules including drugs, toxins, reporter molecules, and receptor ligands; carbohydrate complexes; nucleic acid cleaving complexes; metal chelators (e.g., porphyrins, texaphyrins, crown ethers, etc.); intercalators including hybrid photonuclease/intercalators; crosslinking agents (e.g., photoactive, redox active), and combinations and derivatives thereof. Numerous suitable conjugate moieties, their preparation and linkage to oligomeric compounds are provided, for example, in WO 93/07883 and U.S. Pat. No. 6,395,492, each of which is incorporated herein by reference in its entirety. Oligonucleotide conjugates and their syntheses are also reported in comprehensive reviews by Manoharan in Antisense Drug Technology, Principles, Strategies, and Applications, S. T. Crooke, ed., Ch. 16, Marcel Dekker, Inc., 2001 and Manoharan, Antisense & Nucleic Acid Drug Development, 2002, 12, 103, each of which is incorporated herein by reference in its entirety.

Other lipophilic conjugate moieties include aliphatic groups, such as, for example, straight chain, branched, and cyclic alkyls, alkenyls, and alkynyls. The aliphatic groups can have, for example, 5 to about 50, 6 to about 50, 8 to about 50, or 10 to about 50 carbon atoms. Example aliphatic groups include undecyl, dodecyl, hexadecyl, heptadecyl, octadecyl, nonadecyl, terpenes, bornyl, adamantyl, derivatives thereof and the like. In some embodiments, one or more carbon atoms in the aliphatic group can be replaced by a heteroatom such as O, S, or N (e.g., geranyloxyhexyl). Further suitable lipophilic conjugate moieties include aliphatic derivatives of glycerols such as alkylglycerols, bis(alkyl)glycerols, tris(alkyl)glycerols, monoglycerides, diglycerides, and triglycerides. In some embodiments, the lipophilic conjugate is di-hexyldecyl-rac-glycerol or 1,2-di-O-hexyldecyl-rac-glycerol (Manoharan et al., Tetrahedron Lett., 1995, 36, 3651; Shea, et al., Nuc. Acids Res., 1990, 18, 3777) or phosphonates thereof. Saturated and unsaturated fatty functionalities, such as, for example, fatty acids, fatty alcohols, fatty esters, and fatty amines, can also serve as lipophilic conjugate moieties. In some embodiments, the fatty functionalities can contain from about 6 carbons to about 30 or about 8 to about 22 carbons. Example fatty acids include, capric, caprylic, lauric, palmitic, myristic, stearic, oleic, linoleic, linolenic, arachidonic, eicosenoic acids and the like.

In further embodiments, lipophilic conjugate groups can be polycyclic aromatic groups having from 6 to about 50, 10 to about 50, or 14 to about 40 carbon atoms. Example polycyclic aromatic groups include pyrenes, purines, acridines, xanthenes, fluorenes, phenanthrenes, anthracenes, quinolines, isoquinolines, naphthalenes, derivatives thereof and the like.

Other suitable lipophilic conjugate moieties include menthols, trityls (e.g., dimethoxytrityl (DMT)), phenoxazines, lipoic acid, phospholipids, ethers, thioethers (e.g., hexyl-5-tritylthiol), derivatives thereof and the like. Preparation of lipophilic conjugates of oligomeric compounds are well-described in the art, such as in, for example, Saison-Behmoaras et al., EMBO J., 1991, 10, 1111; Kabanov et al., FEBS Lett., 1990, 259, 327; Svinarchuk et al., Biochimie, 1993, 75, 49; (Mishra et al., Biochim. Biophys. Acta, 1995, 1264, 229, and Manoharan et al., Tetrahedron Lett., 1995, 36, 3651.

Oligomeric compounds containing conjugate moieties with affinity for low density lipoprotein (LDL) can help provide an effective targeted delivery system. High expression levels of receptors for LDL on tumor cells makes LDL an attractive carrier for selective delivery of drugs to these cells (Rump, et al., Bioconjugate Chem., 1998, 9, 341; Firestone, Bioconjugate Chem., 1994, 5, 105; Mishra, et al., Biochim. Biophys. Acta, 1995, 1264, 229). Moieties having affinity for LDL include many lipophilic groups such as steroids (e.g., cholesterol), fatty acids, derivatives thereof and combinations thereof. In some embodiments, conjugate moieties having LDL affinity can be dioleyl esters of cholic acids such as chenodeoxycholic acid and lithocholic acid.

Further cholesterol and related conjugate groups amenable to the present invention include but are not limited to lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-5-tritylthiol (Manoharan et al., Ann. N.Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237) and an octadecylamine or hexylamino-carbonyl-oxycholesterol moiety (Crooke et al., J. Pharmacol. Exp. Ther., 1996, 277, 923-937).

Conjugate moieties can also include vitamins. Vitamins are known to be transported into cells by numerous cellular transport systems. Typically, vitamins can be classified as water soluble or lipid soluble. Water soluble vitamins include thiamine, riboflavin, nicotinic acid or niacin, the vitamin B₆ pyridoxal group, pantothenic acid, biotin, folic acid, the B₁₂ cobamide coenzymes, inositol, choline and ascorbic acid. Lipid soluble vitamins include the vitamin A family, vitamin D, the vitamin E tocopherol family and vitamin K (and phytols). Related compounds include retinoid derivatives such as tazarotene and etretinate.

In some embodiments, the conjugate moiety includes folic acid (folate) and/or one or more of its various forms, such as dihydrofolic acid, tetrahydrofolic acid, folinic acid, pteropolyglutamic acid, dihydrofolates, tetrahydrofolates, tetrahydropterins, 1-deaza, 3-deaza, 5-deaza, 8-deaza, 10-deaza, 1,5-dideaza, 5,10-dideaza, 8,10-dideaza and 5,8-dideaza folate analogs, and antifolates. Folate is involved in the biosynthesis of nucleic acids and therefore impacts the survival and proliferation of cells. Folate cofactors play a role in the one-carbon transfers that are needed for the biosynthesis of pyrimidine nucleosides. Cells therefore have a system of transporting folates into the cytoplasm. Folate receptors also tend to be overexpressed in many human cancer cells, and folate-mediated targeting of oligonucleotides to ovarian cancer cells has been reported (Li, et al., Pharm. Res. 1998, 15, 1540, which is incorporated herein by reference in its entirety). Preparation of Folic Acid Conjugates of Nucleic Acids are Described in, for Example, U.S. Pat. No. 6,528,631, which is incorporated herein by reference in its entirety.

Vitamin conjugate moieties include, for example, vitamin A (retinol) and/or related compounds. The vitamin A family (retinoids), including retinoic acid and retinol, are typically absorbed and transported to target tissues through their interaction with specific proteins such as cytosol retinol-binding protein type II (CRBP-II), retinol-binding protein (RBP), and cellular retinol-binding protein (CRBP). The vitamin A family of compounds can be attached to oligomeric compounds via acid or alcohol functionalities found in the various family members. For example, conjugation of an N-hydroxy succinimide ester of an acid moiety of retinoic acid to an amine function on a linker pendant to an oligonucleotide can result in linkage of vitamin A compound to the oligomeric compound via an amide bond. Also, retinol can be converted to its phosphoramidite, which is useful for 5′ conjugation.

α-Tocopherol (vitamin E) and the other tocopherols (beta through zeta) can be conjugated to oligomeric compounds to enhance uptake because of their lipophilic character. Also, vitamin D, and its ergosterol precursors, can be conjugated to oligomeric compounds through their hydroxyl groups by first activating the hydroxyl groups to, for example, hemisuccinate esters. Conjugation can then be effected directly to the oligomeric compound or to an aminolinker pendant from the oliogmeric compound. Other vitamins that can be conjugated to oligomeric compounds in a similar manner on include thiamine, riboflavin, pyridoxine, pyridoxamine, pyridoxal, deoxypyridoxine. Lipid soluble vitamin K's and related quinone-containing compounds can be conjugated via carbonyl groups on the quinone ring. The phytol moiety of vitamin K can also serve to enhance binding of the oligomeric compounds to cells.

Pyridoxal (vitamin B₆) has specific B₆-binding proteins. The role of these proteins in pyridoxal transport has been studied by Zhang et al., Proc. Natl. Acad. Sci. USA, 1991, 88, 10407. Other pyridoxal family members include pyridoxine, pyridoxamine, pyridoxal phosphate, and pyridoxic acid. Pyridoxic acid, niacin, pantothenic acid, biotin, folic acid and ascorbic acid can be conjugated to oligomeric compounds, for example, using N-hydroxysuccinimide esters that are reactive with aminolinkers located on the oliogmeric compound, as described above for retinoic acid.

Vitamin conjugate moieties can also be used to facilitate the targeting of specific cells or tissues. For example, vitamin D and analogs thereof can assist in transporting conjugated oligomeric compounds to keratinocytes, dermal fibroblasts, and other cells containing vitamin D₃ nuclear receptors. Additionally, Vitamin A and other retinoids can be used to target cells with retinoid X receptors. Accordingly, vitamin-containing conjugate moieties can be useful in treating, for example, skin disorders such as psoriasis.

Conjugate moieties can also include polymers. Polymers can provide added bulk and various functional groups to affect permeation, cellular transport, and localization of the conjugated oligomeric compound. For example, increased hydrodynamic radius caused by conjugation of an oligomeric compound with a polymer can help prevent entry into the nucleus and encourage localization in the cytoplasm. In some embodiments, the polymer does not substantially reduce cellular uptake or interfere with hybridization to a complementary strand or other target. In further embodiments, the conjugate polymer moiety has, for example, a molecular weight of less than about 40, less than about 30, or less than about 20 kDa. Additionally, polymer conjugate moieties can be water-soluble and optionally further comprise other conjugate moieties such as peptides, carbohydrates, drugs, reporter groups, or further conjugate moieties.

In some embodiments, polymer conjugates include polyethylene glycol (PEG) and copolymers and derivatives thereof. Conjugation to PEG has been shown to increase nuclease stability of an oligomeric compound. PEG conjugate moieties can be of any molecular weight including for example, about 100, about 500, about 1000, about 2000, about 5000, about 10,000 and higher. In one particularly preferred embodiment the molecular weight of the PEG group is 20,000 daltons. In some embodiments, the PEG conjugate moieties contains at least 4, at least 5, at least 6, at least 7, at least 8, at least 9, at least 10, at least 15, at least 20, or at least 25 ethylene glycol residues. In further embodiments, the PEG conjugate moiety contains from about 4 to about 10, about 4 to about 8, about 5 to about 7, or about 6 ethylene glycol residues. The PEG conjugate moiety can also be modified such that a terminal hydroxyl is replaced by alkoxy, carboxy, acyl, amido, or other functionality. Other conjugate moieties, such as reporter groups including, for example, biotin or fluorescein can also be attached to a PEG conjugate moiety. Copolymers of PEG are also suitable as conjugate moieties.

Preparation and biological activity of polyethylene glycol conjugates of oligonucleotides are described, for example, in Bonora, et al., Nucleosides Nucleotides, 1999, 18, 1723; Bonora, et al., Farmaco, 1998, 53, 634; Efimov, Bioorg. Khim. 1993, 19, 800; and Jaschke, et al., Nucleic Acids Res., 1994, 22, 4810. Further example PEG conjugate moieties and preparation of corresponding conjugated oligomeric compounds is described in, for example, U.S. Pat. Nos. 4,904,582 and 5,672,662, each of which is incorporated by reference herein in its entirety. Oligomeric compounds conjugated to one or more PEG moieties are available commercially.

Other polymers suitable as conjugate moieties include polyamines, polypeptides, polymethacrylates (e.g., hydroxylpropyl methacrylate (HPMA)), poly(L-lactide), poly(DL lactide-co-glycolide (PGLA), polyacrylic acids, polyethylenimines (PEI), polyalkylacrylic acids, polyurethanes, polyacrylamides, N-alkylacrylamides, polyspermine (PSP), polyethers, cyclodextrins, derivatives thereof and co-polymers thereof. Many polymers, such as PEG and polyamines have receptors present in certain cells, thereby facilitating cellular uptake. Polyamines and other amine-containing polymers can exist in protonated form at physiological pH, effectively countering an anionic backbone of some oligomeric compounds, effectively enhancing cellular permeation. Some example polyamines include polypeptides (e.g., polylysine, polyornithine, polyhistadine, polyarginine, and copolymers thereof), triethylenetetraamine, spermine, polyspermine, spermidine, synnorspermidine, C-branched spermidine, and derivatives thereof. Preparation and biological activity of polyamine conjugates are described, for example, in Guzaev, et al., Bioorg. Med. Chem. Lett., 1998, 8, 3671; Corey, et al., J. Am. Chem. Soc., 1995, 117, 9373; and Prakash, et al., Bioorg. Med. Chem. Lett. 1994, 4, 1733. Example polypeptide conjugates of oligonucleotides are provided in, for example, Wei, et al., Nucleic Acids Res., 1996, 24, 655 and Zhu, et al., Antisense Res. Dev., 1993, 3, 265. Dendrimeric polymers can also be used as conjugate moieties, such as described in U.S. Pat. No. 5,714,166, which is incorporated herein by reference in its entirety.

As discussed above for polyamines and related polymers, other amine-containing moieties can also serve as suitable conjugate moieties due to, for example, the formation of cationic species at physiological conditions. Example amine-containing moieties include 3-aminopropyl, 3-(N,N-dimethylamino)propyl, 2-(2-(N,N-dimethylamino)ethoxy)ethyl, 2-(N-(2-aminoethyl)-N-methylaminooxy)ethyl, 2-(1-imidazolyl)ethyl, and the like. The G-clamp moiety can also serve as an amine-containing conjugate moiety (Lin, et al., J. Am. Chem. Soc., 1998, 120, 8531).

Conjugate moieties can also include peptides. Suitable peptides can have from 2 to about 30, 2 to about 20, 2 to about 15, or 2 to about 10 amino acid residues. Amino acid residues can be naturally or non-naturally occurring, including both D and L isomers.

In some embodiments, peptide conjugate moieties are pH sensitive peptides such as fusogenic peptides. Fusogenic peptides can facilitate endosomal release of agents such as oligomeric compounds to the cytoplasm. It is believed that fusogenic peptides change conformation in acidic pH, effectively destabilizing the endosomal membrane thereby enhancing cytoplasmic delivery of endosomal contents. Examples of fusogenic peptides include peptides derived from polymyxin B, influenza HA2, GAL4, KALA, EALA, melittin-derived peptide, α-helical peptide or Alzheimer β-amyloid peptide, and the like. Preparation and biological activity of oligonucleotides conjugated to fusogenic peptides are described in, for example, Bongartz, et al., Nucleic Acids Res., 1994, 22, 4681 and U.S. Pat. Nos. 6,559,279 and 6,344,436.

Other peptides that can serve as conjugate moieties include delivery peptides which have the ability to transport relatively large, polar molecules (including peptides, oligonucleotides, and proteins) across cell membranes. Examples of delivery peptides include Tat peptide from HIV Tat protein and Ant peptide from Drosophila antenna protein. Conjugation of Tat and Ant with oligonucleotides is described in, for example, Astriab-Fisher, et al., Biochem. Pharmacol, 2000, 60, 83. These and other delivery peptides that can be used as conjugate moieties are provided below in Table I.

TABLE I

| SEQ ID | Delivery Peptide | |
|---|---|---|
| NO: | Sequence | Source |
| 10 | RQIKIWFQNRRMKWKK | Antennapodia helix 3 Antp-HD |
| 11 | GRKKRRQRRRPPQ | HIV Tat fragment |
| 12 | GWTLNSAGYLLGPINLKALAAL | Transporton: chimeric galanin |
| | AKKIL | and mastoporan |
| 13 | DAATATRGRSAASRPTERPRAP | HSV VP22 |
| | ARSASRPRRPVE | |
| 14 | KLALKLALKALKAALKLA | Amphiphilic peptide |
| 15 | GALFLGWLGAAGSTMGAWSQP | Signal sequence based peptide I |
| | KKKRKV | |
| 16 | AAVALLPAVLLALLAP | Signal sequence based peptide II |
| 17 | PKKKRKV | SV40 antigen T nuclear |
| | localization signal | |
| 18 | MLFY | Platelet activating factor |
| | | receptor of neutrophils |
| 19 | PQRRNRSRRRRFRGQ | FXR2P |
| 20 | IMRRRGL | angiogenin |
| 21 | LQLPPLERLTL | HIV-1 Rev |
| 22 | ELALKLAGLDI | PKI-α |
| 23 | DLQKKLEELEL | MAPKK |
| 24 | ALPHAIMRLDLA | actin |
| 25 | PKLKKRKV | simian virus 40 large tumor |
| | | antigen |
| 26 | ALWKTLLKKVLKA | Dermaseptin |
| 27 | dPheCysPhedTrpLysThrCysThr | Octatrate (Cys-Cys linked) |
| 28 | CysGlyAsnLysArgThrArgGlyCys | Lyp-1 (Cys-Cys linked) |
| 29 | GlyHisLysAlaLysGlyProArgLys | B-6 |

Conjugated delivery peptides can help control localization of oligomeric compounds to specific regions of a cell, including, for example, the cytoplasm, nucleus, nucleolus, and endoplasmic reticulum (ER). Nuclear localization can be effected by conjugation of a nuclear localization signal (NLS). In contrast, cytoplasmic localization can be facilitated by conjugation of a nuclear export signal (NES).

Peptides suitable for localization of conjugated oligomeric compounds in the nucleus include, for example, N,N-dipalmitylglycyl-apo E peptide or N,N-dipalmitylglycyl-apolipoprotein E peptide (dpGapoE) (Liu, et al., Arterioscler. Thromb. Vasc. Biol., 1999, 19, 2207; Chaloin, et al., Biochem. Biophys. Res. Commun., 1998, 243, 601). Nucleus or nucleolar localization can also be facilitated by peptides having arginine and/or lysine rich motifs, such as in HIV-1 Tat, FXR2P, and angiogenin derived peptides (Lixin, et al., Biochem. Biophys. Res. Commun., 2001, 284, 185). Additionally, the nuclear localization signal (NLS) peptide derived from SV40 antigen T (Branden, et al., Nature Biotech, 1999, 17, 784) can be used to deliver conjugated oligomeric compounds to the nucleus of a cell. Other suitable peptides with nuclear or nucleolar localization properties are described in, for example, Antopolsky, et al., Bioconjugate Chem., 1999, 10, 598; Zanta, et al., Proc. Natl. Acad. Sci. USA, 1999 (simian virus 40 large tumor antigen); Hum. Mol. Genetics, 2000, 9, 1487; and FEBS Lett., 2002, 532, 36).

In some embodiments, the delivery peptide for nucleus or nucleolar localization comprises at least three consecutive arginine residues or at least four consecutive arginine residues. Nuclear localization can also be facilitated by peptide conjugates containing RS, RE, or RD repeat motifs (Cazalla, et al., Mol. Cell. Biol., 2002, 22, 6871). In some embodiments, the peptide conjugate contains at least two RS, RE, or RD motifs.

Localization of oligomeric compounds to the ER can be effected by, for example, conjugation to the signal peptide KDEL (SEQ ID NO: 30) (Arar, et al., Bioconjugate Chem., 1995, 6, 573; Pichon, et al., Mol. Pharmacol. 1997, 51, 431).

Cytoplasmic localization of oligomeric compounds can be facilitated by conjugation to peptides having, for example, a nuclear export signal (NES) (Meunier, et al., Nucleic Acids Res., 1999, 27, 2730). NES peptides include the leucine-rich NES peptides derived from HIV-1 Rev (Henderson, et al., Exp. Cell Res., 2000, 256, 213), transcription factor III A, MAPKK, PKI-alpha, cyclin BI, and actin (Wada, et al., EMBO J., 1998, 17, 1635) and related proteins. Antimicrobial peptides, such as dermaseptin derivatives, can also facilitate cytoplasmic localization (Hariton-Gazal, et al., Biochemistry, 2002, 41, 9208). Peptides containing RG and/or KS repeat motifs can also be suitable for directing oligomeric compounds to the cytoplasm. In some embodiments, the peptide conjugate moieties contain at least two RG motifs, at least two KS motifs, or at least one RG and one KS motif.

As used throughout, “peptide” includes not only the specific molecule or sequence recited herein (if present), but also includes fragments thereof and molecules comprising all or part of the recited sequence, where desired functionality is retained. In some embodiments, peptide fragments contain no fewer than 6 amino acids. Peptides can also contain conservative amino acid substitutions that do not substantially change its functional characteristics. Conservative substitution can be made among the following sets of functionally similar amino acids: neutral-weakly hydrophobic (A, G, P, S, T), hydrophilic-acid amine (N, D, Q, E), hydrophilic-basic (I, M, L, V), and hydrophobic-aromatic (F, W, Y). Peptides also include homologous peptides. Homology can be measured according to percent identify using, for example, the BLAST algorithm (default parameters for short sequences). For example, homologous peptides can have greater than 50, 60, 70, 80, 90, 95, or 99 percent identity. Methods for conjugating peptides to oligomeric compounds such as oligonucleotides are described in, for example, U.S. Pat. No. 6,559,279, which is incorporated herein by reference in its entirety.

Like delivery peptides, nucleic acids can also serve as conjugate moieties that can affect localization of conjugated oligomeric compounds in a cell. For example, nucleic acid conjugate moieties can contain poly A, a motif recognized by poly A binding protein (PABP), which can localize poly A-containing molecules in the cytoplasm (Gorlach, et al., Exp. Cell Res., 1994, 211, 400. In some embodiments, the nucleic acid conjugate moiety contains at least 3, at least 4, at least 5, at least 6, at least 7, at least 8, at least 9, at least 10, at least 15, at least 20, and at least 25 consecutive A bases. The nucleic acid conjugate moiety can also contain one or more AU-rich sequence elements (AREs). AREs are recognized by ELAV family proteins which can facilitate localization to the cytoplasm (Bollig, et al., Biochem. Bioophys. Res. Commun., 2003, 301, 665). Example AREs include UUAUUUAUU and sequences containing multiple repeats of this motif. In other embodiments, the nucleic acid conjugate moiety contains two or more AU or AUU motifs. Similarly, the nucleic acid conjugate moiety can also contain one or more CU-rich sequence elements (CREs) (Wein, et al., Eur. J. Biochem., 2003, 270, 350) which can bind to proteins HuD and/or HuR of the ELAV family of proteins. As with AREs, CREs can help localize conjugated oligomeric compounds to the cytoplasm. In some embodiments, the nucleic acid conjugate moiety contains the motif (CUUU)ₙ, wherein, for example, n can be 1 to about 20, 1 to about 15, or 1 to about 11. The (CUUU)ₙ motif can optionally be followed or preceded by one or more U. In some embodiments, n is about 9 to about 12 or about 11.

The nucleic acid conjugate moiety can also include substrates of hnRNP proteins (heterogeneous nuclear ribonucleoprotein), some of which are involved in shuttling nucleic acids between the nucleus and cytoplasm. (e.g., nhRNP A1 and nhRNP K; see, e.g., Mili, et al., Mol. Cell. Biol., 2001, 21, 7307). Some example hnRNP substrates include nucleic acids containing the sequence UAGGA/U or (GG)ACUAGC(A). Other nucleic acid conjugate moieties can include Y strings or other tracts that can bind to, for example, hnRNP I. In some embodiments, the nucleic acid conjugate can contain at least 3, at least 4, at least 5, at least 6, at least 7, at least 8, at least 9, at least 10, at least 15, at least 20, and at least 25 consecutive pyrimidine bases. In other embodiments the nucleic acid conjugate can contain greater than 50, greater than 60, greater than 70, greater than 80, greater than 90, or greater than 95 percent pyrimidine bases.

Other nucleic acid conjugate moieties can include pumilio (puf protein) recognition sequences such as described in Wang, et al., Cell, 2002, 110, 501. Example pumilio recognition sequences can include UGUANAUR, where N can be any base and R can be a purine base.

Localization to the cytoplasm can be facilitated by nucleic acid conjugate moieties containing AREs and/or CREs. Nucleic acid conjugate moieties serving as substrates of hnRNPs can facilitate localization of conjugated oligomeric compounds to the cytoplasm (e.g., hnRNP A1 or K) or nucleus (e.g., hnRNP I). Additionally, nucleus localization can be facilitated by nucleic acid conjugate moieties containing polypyrimidine tracts.

Many drugs, receptor ligands, toxins, reporter molecules, and other small molecules can serve as conjugate moieties. Small molecule conjugate moieties often have specific interactions with certain receptors or other biomolecules, thereby allowing targeting of conjugated oligomeric compounds to specific cells or tissues. Example small molecule conjugate moieties include mycophenolic acid (inhibitor of inosine-5′-monophosphate dihydrogenase; useful for treating psoriasis and other skin disorders), curcumin (has therapeutic applications to psoriasis, cancer, bacterial and viral diseases). In further embodiments, small molecule conjugate moieties can be ligands of serum proteins such as human serum albumin (HSA). Numerous ligands of HSA are known and include, for example, arylpropionic acids, ibuprofen, warfarin, phenylbutazone, suprofen, carprofen, fenfufen, ketoprofen, aspirin, indomethacin, (S)-(+)-pranoprofen, dansylsarcosine, 2,3,5-triiodobenzoic acid, flufenamic acid, folinic acid, benzothiadiazide, chlorothiazide, diazepines, indomethicin, barbituates, cephalosporins, sulfa drugs, antibacterials, antibiotics (e.g., puromycin and pamamycin), and the like. Oligonucleotide-drug conjugates and their preparation are described in, for example, WO 00/76554, which is incorporated herein by reference in its entirety. Oligonucleotide-drug conjugates and their preparation are also described in U.S. patent application Ser. No. 09/334,130, which is incorporated herein by reference in its entirety.

In yet further embodiments, small molecule conjugates can target or bind certain receptors or cells. T-cells are known to have exposed amino groups that can form Schiff base complexes with appropriate molecules. Thus, small molecules containing functional groups such as aldehydes that can interact or react with exposed amino groups can also be suitable conjugate moieties. Tucaresol and related compounds can be conjugated to oligomeric compounds in such as way as to leave the aldehyde free to interact with T-cell targets. Interaction of tucaresol with T-cells in believed to result in therapeutic potentiation of the immune system by Schiff-base formation (Rhodes, et al., Nature, 1995, 377, 6544).

Reporter groups that are suitable as conjugate moieties include any moiety that can be detected by, for example, spectroscopic means. Example reporter groups include dyes, fluorophores, phosphors, radiolabels, and the like. In some embodiments, the reporter group is biotin, flourescein, rhodamine, coumarin, or related compounds. Reporter groups can also be attached to other conjugate moieties.

Other conjugate moieties can include proteins, subunits, or fragments thereof. Proteins include, for example, enzymes, reporter enzymes, antibodies, receptors, and the like. In some embodiments, protein conjugate moieties can be antibodies or fragments thereof (Kuijpers, et al., Bioconjugate Chem., 1993, 4, 94). Antibodies can be designed to bind to desired targets such as tumor and other disease-related antigens. In further embodiments, protein conjugate moieties can be serum proteins such as HAS or glycoproteins such as asialoglycoprotein (Rajur, et al., Bioconjugate Chem., 1997, 6, 935). In yet further embodiments, oligomeric compounds can be conjugated to RNAi-related proteins, RNAi-related protein complexes, subunits, and fragments thereof. For example, oligomeric compounds can be conjugated to Dicer or RISC.

Other conjugate moieties can include, for example, oligosaccharides and carbohydrate clusters such as Tyr-Glu-Glu-(aminohexyl GalNAc)₃ (YEE(ahGalNAc)₃; a glycotripeptide that binds to Gal/GalNAc receptors on hepatocytes, see, e.g., Duff, et al., Methods Eanzymol., 2000, 313, 297); lysine-based galactose clusters (e.g., L₃G₄; Biessen, et al., Dev. Cardovasc. Med., 1999, 214); and cholane-based galactose clusters (e.g., carbohydrate recognition motif for asialoglycoprotein receptor). Further suitable conjugates can include oligosaccharides that can bind to carbohydrate recognition domains (CRD) found on the asiologlycoprotein-receptor (ASGP-R). Example conjugate moieties containing oligosaccharides and/or carbohydrate complexes are provided in U.S. Pat. No. 6,525,031, which is incorporated herein by reference in its entirey.

Intercalators and minor groove binders (MGBs) can also be suitable as conjugate moieties. In some embodiments, the MGB can contain repeating DPI (1,2-dihydro-3H-pyrrolo[2,3-e]indole-7-carboxylate) subunits or derivatives thereof (Lukhtanov, et al., Bioconjugate Chem., 1996, 7, 564 and Afonina, et al., Proc. Natl. Acad. Sci. USA, 1996, 93, 3199). Suitable intercalators include, for example, polycyclic aromatics such as naphthalene, pyrene, phenanthridine, benzophenanthridine, phenazine, anthraquinone, acridine, and derivatives thereof. Hybrid intercalator/ligands include the photonuclease/intercalator ligand 6-[[[9-[[6-(4-nitrobenzamido)hexyl]amino]acridin-4-yl]carbonyl]amino]hexan oyl-pentafluorophenyl ester. This compound is both an acridine moiety that is an intercalator and a p-nitro benzamido group that is a photonuclease.

In further embodiments, cleaving agents can serve as conjugate moieties. Cleaving agents can facilitate degradation of target, such as target nucleic acids, by hydrolytic or redox cleavage mechamisms. Cleaving groups that can be suitable as conjugate moieties include, for example, metallocomplexes, peptides, amines, enzymes, and constructs containing constituents of the active sites of nucleases such as imidazole, guanidinium, carboxyl, amino groups, etx.). Example metallocomplexes include, for example, Cu-terpyridyl complexes, Fe-porphyrin complexes, Ru-complexes, and lanthanide complexes such as various Eu(III) complexes (Hall, et al., Chem. Biol., 1994, 1, 185; Huang, et al., J. Biol. Inorg. Chem., 2000, 5, 85; and Baker, et al., Nucleic Acids Res., 1999, 27, 1547). Other metallocomplexes with cleaving properties include metalloporphyrins and derivatives thereof. Example peptides with target cleaving properties include zinc fingers (U.S. Pat. No. 6,365,379; Lima, et al., Proc. Natl. Acad. Sci. USA, 1999, 96, 10010). Example constructs containing nuclease active site constituents include bisimiazole and histamine.

Cross-linking agents can also serve as conjugate moieties. Cross-linking agents facilitate the covalent linkage of the conjugated oligomeric compounds with other compounds. In some embodiments, cross-linking agents can covalently link double-stranded nucleic acids, effectively increasing duplex stability and modulating pharmacokinetic properties. In some embodiments, cross-linking agents can be photoactive or redox active. Example cross-linking agents include psoralens which can facilitate interstrand cross-linking of nucleic acids by photoactivation (Lin, et al., Faseb J., 1995, 9, 1371). Other cross-linking agents include, for example, mitomycin C and analogs thereof (Maruenda, et al., Bioconjugate Chem., 1996, 7, 541; Maruenda, et al., Anti-Cancer Drug Des., 1997, 12, 473; and Huh, et al., Bioconjugate Chem., 1996, 7, 659). Cross-linking mediated by mitomycin C can be effected by reductive activation, such as, for example, with biological reductants (e.g., NADPH-cytochrome c reductase/NADPH system). Further photo-crosslinking agents include aryl azides such as, for example, N-hydroxysucciniimidyl-4-azidobenzoate (HSAB) and N-succinimidyl-6(-4′-azido-2′-nitrophenyl-amino)hexanoate (SANPAH). Aryl azides conjugated to oligonucleotides effect crosslinking with nucleic acids and proteins upon irradiation. They can also crosslink with carrier proteins (such as KLH or BSA).

Other suitable conjugate moieties include, for example, polyboranes, carboranes, metallopolyboranes, metallocarborane, derivatives thereof and the like (see, e.g., U.S. Pat. No. 5,272,250, which is incorporated herein by reference in its entirety).

The term “bivalent tethering moiety” “tether” as used herein describes the connecting group that derives from a tether precursor having two reactive functional groups that covalently attaches a conjugate group to a pyrrolidinyl group of the invention. Any compound having one group that can react with and form a covalent bond with the endocyclic amino group of the pyrrolidinyl group and having a second group that can react with and form a covalent bond with a selected conjugate group can function as the tether precursor. In one aspect the tether precursor reacts with the pyrrolidinyl endocyclic amino group to form an amide bond (pyrrolidinyl amine-C(═O)-tether). In another aspect the tether precursor reacts with a conjugate group to form an amide linkage having the formula (tether-N(Rₐ)—C(═O)-conjugate) where the C(═O) is part of a reactive group on the conjugate and the N(Rₐ) is from a reactive group on the tether precursor) wherein Rₐ is H, lower alkyl or substituted lower alkyl. Preferred reactive sites on the tether precursor include carboxyl for reaction with the pyrrolidinyl endocyclic amino group to form an amide bond and amino for reacting with a carboxyl group supplied by the conjugate group to form an amide bond.

In addition to the two reactive sites preferably on the two ends of the tether precursor there is also a central region connecting the two reactive sites that comprises essentially an alkyl region or a substituted alkyl region where the alkyl methylene groups can be separated by heteroatoms selected from N(Rₐ), O and S. A couple of non limiting examples of some tethering groups include: —C(═O)—CH₂—O—(CH₂)₂—O—(CH₂)₂—N(Rₐ)— and —C(═O)—(CH₂)₅—N(Rₐ)—.

Conjugate moieties can be attached to the pyrrolidinyl groups of the invention directly or through a tether. In some embodiments, the tether comprises a chain structure or an oligomer of repeating units such as ethylene glyol or amino acid units. The linker can have at least two functionalities, one for attaching to the pyrrolidinyl group and the other for attaching to the conjugate moiety. Examples of tether chemistries amenable to attachment of conjugate groups to pyrrolidinyl groups include functional groups on the ends of the tether that are electrophilic for reacting with nucleophilic groups on the pyrrolidinyl group and the conjugate group or alternatively the nucleophilic groups can be located on the tether and the electrophilic groups on the pyrrolidinyl group and conjugate group. In some embodiments, tether functional groups include amino, hydroxyl, carboxylic acid, thiol, phosphoramidate, phosphate, phosphite, unsaturations (e.g., double or triple bonds), and the like. Some example tethers include 8-amino-3,6-dioxaoctanoic acid (ADO), succinimidyl 4-(N-maleimidomethyl)cyclohexane-1-carboxylate (SMCC), 6-aminohexanoic acid (AHEX or AHA), 6-aminohexyloxy, 4-aminobutyric acid, 4-aminocyclohexylcarboxylic acid, succinimidyl 4-(N-maleimidomethyl)cyclohexane-1-carboxy-(6-amido-caproate) (LCSMCC), succinimidyl m-maleimido-benzoylate (MBS), succinimidyl N-ε-maleimido-caproylate (EMCS), succinimidyl 6-(β-maleimido-propionamido) hexanoate (SMPH), succinimidyl N-(α-maleimido acetate) (AMAS), succinimidyl 4-(p-maleimidophenyl)butyrate (SMPB), β-alanine (β-ALA), phenylglycine (PHG), 4-aminocyclohexanoic acid (ACHC), β-(cyclopropyl) alanine (β-CYPR), amino dodecanoic acid (ADC), alylene diols, polyethylene glycols, amino acids, and the like.

Any of the above groups can be used as a single linker or in combination with one or more further tethers.

Tethers and their use in preparation of conjugates of oligomeric compounds are provided throughout the art such as in WO 96/11205 and WO 98/52614 and U.S. Pat. Nos. 4,948,882; 5,525,465; 5,541,313; 5,545,730; 5,552,538; 5,580,731; 5,486,603; 5,608,046; 4,587,044; 4,667,025; 5,254,469; 5,245,022; 5,112,963; 5,391,723; 5,510,475; 5,512,667; 5,574,142; 5,684,142; 5,770,716; 6,096,875; 6,335,432; and 6,335,437, each of which is incorporated by reference in its entirety.

Representative United States patents that teach the preparation of oligonucleotide conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,149,782; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928; 5,672,662; 5,688,941; 5,714,166; 6,153,737; 6,172,208; 6,300,319; 6,335,434; 6,335,437; 6,395,437; 6,444,806; 6,486,308; 6,525,031; 6,528,631; 6,559,279; each of which is herein incorporated by reference.

In some embodiments, conjugate moieties can be attached to heterocyclic base moieties (e.g., purines and pyrimidines), monomeric subunits (e.g., sugar moieties), or monomeric subunit linkages (e.g., phosphodiester linkages) of nucleic acid molecules. Conjugation to purines or derivatives thereof can occur at any position including, endocyclic and exocyclic atoms. In some embodiments, the 2-, 6-, 7-, or 8-positions of a purine base are attached to a conjugate moiety. Conjugation to pyrimidines or derivatives thereof can also occur at any position. In some embodiments, the 2-, 5-, and 6-positions of a pyrimidine base can be substituted with a conjugate moiety. Conjugation to sugar moieties of nucleosides can occur at any carbon atom. Example carbon atoms of a sugar moiety that can be attached to a conjugate moiety include the 2′, 3′, and 5′ carbon atoms. The 1′ position can also be attached to a conjugate moiety, such as in an abasic residue. Internucleosidic linkages can also bear conjugate moieties. For phosphorus-containing linkages (e.g., phosphodiester, phosphorothioate, phosphorodithiotate, phosphoroamidate, and the like), the conjugate moiety can be attached directly to the phosphorus atom or to an O, N, or S atom bound to the phosphorus atom. For amine- or amide-containing internucleosidic linkages (e.g., PNA), the conjugate moiety can be attached to the nitrogen atom of the amine or amide or to an adjacent carbon atom.

The term “bifunctional linking moiety,” as used herein describes a bifunctional linker that covalently attaches the pyrrolidinyl group to a support medium. A number of bifunctional linking moieties are know in the art such as for example a succinyl —C(═O)—(CH₂)₂—C(═O)— group. The precursor of the bifunctional linking moiety has one reactive site that reacts and forms a covalent bond with a support medium and a second reactive site that reacts and forms a covalent bond with a pyrrolidinyl group. A wide variety of precursor bifunctional linking moieties are amenable to the present invention. A preferred precursor bifunctional linking moiety includes a carboxyl, ester or anhydride group for forming an ester linkage with a hydroxyl group on the pyrrolidinyl group and further includes a second reactive group such as a carboxyl or ester group for forming an amide or ester linkage with a support medium. A preferred precursor bifunctional linking moiety includes succinic anhydride.

Suitable reagents for preparing “support medium-OCO-Q-CO-pyrrolidinyl” include diacids (HO₂C-Q-CO₂H). Particularly suitable diacids include malonic acid (Q is methylene), succinic acid (Q is 1,2-ethylene), glutaric acid, adipic acid, pimelic acid, and phthalic acid. Other suitable reagents include diacid anhydrides. Particularly suitable diacid anhydrides include malonic anhydride, succinic anhydride, glutaric anhydride, adipic anhydride, pimelic anhydride, and phthalic anhydride. Other suitable reagents include diacid esters, diacid halides, etc. One especially preferred reagent is succinic anhydride.

In one aspect the bifunctional linking moiety makes a covalent attachment to a support medium via a terminal carboxylic acid thereby forming an amide linkage with an amine reagent on the support surface. In other aspects, the terminal carboxylic acid forms an ester with an OH group on the support medium. In some embodiments, the terminal carboxylic acid may be replaced with a terminal acid halide, acid ester, acid anhydride, etc. Specific acid halides include carboxylic chlorides, bromides and iodides. Specific esters include methyl, ethyl, and other C₁-C₁₀ alkyl esters. Specific anhydrides include formyl, acetyl, propanoyl, and other C₁-C₁₀ alkanoyl esters.

Oligomeric compounds of the present invention can also be modified to have one or more stabilizing groups that are generally attached to one or both termini to enhance properties such as for example nuclease stability. Included in stabilizing groups are cap structures. The terms “cap structure” or “terminal cap moiety,” as used herein, refer to chemical modifications, which can be attached to one or both of the termini of an oligomeric compound. These terminal modifications protect the oligomeric compounds having terminal nucleic acid moieties from exonuclease degradation, and can help in delivery and/or localization within a cell. The cap can be present at the 5′-terminus (5′-cap) or at the 3′-terminus (3′-cap) or can be present on both termini. In non-limiting examples, the 5′-cap includes inverted abasic residue (moiety), 4′,5′-methylene nucleotide; 1-(beta-D-erythrofuranosyl) nucleotide, 4′-thio nucleotide, carbocyclic nucleotide; 1,5-anhydrohexitol nucleotide; L-nucleotides; alpha-nucleotides; modified base nucleotide; phosphorodithioate linkage; threo-pentofuranosyl nucleotide; acyclic 3′,4′-seco nucleotide; acyclic 3,4-dihydroxybutyl nucleotide; acyclic 3,5-dihydroxypentyl riucleotide, 3′-3′-inverted nucleotide moiety; 3′-3′-inverted abasic moiety; 3′-2′-inverted nucleotide moiety; 3′-2′-inverted abasic moiety; 1,4-butanediol phosphate; 3′-phosphoramidate; hexylphosphate; aminohexyl phosphate; 3′-phosphate; 3′-phosphorothioate; phosphorodithioate; or bridging or non-bridging methylphosphonate moiety (for more details see Wincott et al., International PCT publication No. WO 97/26270, incorporated by reference herein).

Particularly preferred 3′-cap structures of the present invention include, for example 4′,5′-methylene nucleotide; 1-(beta-D-erythrofuranosyl) nucleotide; 4′-thio nucleotide, carbocyclic nucleotide; 5′-amino-alkyl phosphate; 1,3-diamino-2-propyl phosphate, 3-aminopropyl phosphate; 6-aminohexyl phosphate; 1,2-aminododecyl phosphate; hydroxypropyl phosphate; 1,5-anhydrohexitol nucleotide; L-nucleotide; alpha-nucleotide; modified base nucleotide; phosphorodithioate; threo-pentofuranosyl nucleotide; acyclic 3′,4′-seco nucleotide; 3,4-dihydroxybutyl nucleotide; 3,5-dihydroxy-pentyl nucleotide, 5′-5′-inverted nucleotide moiety; 5′-5′-inverted abasic moiety; 5′-phosphoramidate; 5′-phosphorothioate; 1,4-butanediol phosphate; 5′-amino; bridging and/or non-bridging 5′-phosphoramidate, phosphorothioate and/or phosphorodithioate, bridging or non bridging methylphosphonate and 5′-mercapto moieties (for more details see Beaucage and Tyer, 1993, Tetrahedron 49, 1925).

Further 3′ and 5′-stabilizing groups that can be used to cap one or both ends of an oligomeric compound to impart nuclease stability include those disclosed in WO 03/004602.

The term “alkyl,” as used herein, refers to a saturated straight or branched hydrocarbon radical containing up to twenty four carbon atoms. Examples of alkyl groups include, but are not limited to, methyl, ethyl, propyl, butyl, isopropyl, n-hexyl, octyl, decyl, dodecyl and the like. Alkyl groups typically include from 1 to about 24 carbon atoms, more typically from 1 to about 12 carbon atoms with from 1 to about 6 carbon atoms being more preferred. The term “lower alkyl” as used herein includes from 1 to about 12 carbon atoms. Alkyl groups as used herein may optionally include one or more further substitutent groups.

The term “alkenyl,” as used herein, refers to a straight or branched hydrocarbon chain radical containing up to twenty four carbon atoms having at least one carbon-carbon double bond. Examples of alkenyl groups include, but are not limited to, ethenyl, propenyl, butenyl, 1-methyl-2-buten-1-yl, dienes such as 1,3-butadiene and the like. Alkenyl groups typically include from 2 to about 24 carbon atoms, more typically from 2 to about 12 carbon atoms with from 2 to about 6 carbon atoms being more preferred. Alkenyl groups as used herein may optionally include one or more further substitutent groups.

The term “alkynyl,” as used herein, refers to a straight or branched hydrocarbon radical containing up to twenty four carbon atoms and having at least one carbon-carbon triple bond. Examples of alkynyl groups include, but are not limited to, ethynyl, 1-propynyl, 1-butynyl, and the like. Alkynyl groups typically include from 2 to about 24 carbon atoms, more typically from 2 to about 12 carbon atoms with from 2 to about 6 carbon atoms being more preferred. Alkynyl groups as used herein may optionally include one or more further substitutent groups.

The term “aliphatic,” as used herein, refers to a straight or branched hydrocarbon radical containing up to twenty four carbon atoms wherein the saturation between any two carbon atoms is a single, double or triple bond. An aliphatic group preferably contains from 1 to about 24 carbon atoms, more typically from 1 to about 12 carbon atoms with from 1 to about 6 carbon atoms being more preferred. The straight or branched chain of an aliphatic group may be interrupted with one or more heteroatoms that include nitrogen, oxygen, sulfur and phosphorus. Such aliphatic groups interrupted by heteroatoms include without limitation polyalkoxys, such as polyalkylene glycols, polyamines, and polyimines, for example. Aliphatic groups as used herein may optionally include further substitutent groups.

The term “alkoxy,” as used herein, refers to a radical formed between an alkyl group and an oxygen atom wherein the oxygen atom is used to attach the alkoxy group to a parent molecule. Examples of alkoxy groups include, but are not limited to, methoxy, ethoxy, propoxy, isopropoxy, n-butoxy, sec-butoxy, tert-butoxy, n-pentoxy, neopentoxy, n-hexoxy and the like. Alkoxy groups as used herein may optionally include further substitutent groups.

The terms “halo” and “halogen,” as used herein, refer to an atom selected from fluorine, chlorine, bromine and iodine.

The terms “aryl” and “aromatic,” as used herein, refer to a mono- or polycyclic carbocyclic ring system radical having one or more aromatic rings. Examples of aryl groups include, but not limited to, phenyl, naphthyl, tetrahydronaphthyl, indanyl, idenyl and the like. Aryl groups as used herein may optionally include further substitutent groups.

The terms “aralkyl” and “arylalkyl,” as used herein, refer to a radical formed between an alkyl group and an aryl group wherein the alkyl group is used to attach the aralkyl group to a parent molecule. Examples include, but are not limited to, benzyl, phenethyl and the like. Aralkyl groups as used herein may optionally include further substitutent groups attached to the alkyl, the aryl or both groups that form the radical group.

The term “alicyclic,” as used herein, refers to a radical monocyclic or polycyclic saturated hydrocarbon ring or ring system. Examples include, but are not limited to, cyclopropyl, cyclobutyl, cyclopentyl, cyclohexyl, bicyclo [2.2.1]heptyl, bicyclo [2.2.2] octyl and the like. Alicyclic groups as used herein may optionally include further substitutent groups.

The term “heterocyclic,” as used herein, refers to a radical mono-, or poly-cyclic ring system that includes at least one heteroatom and is unsaturated, partially saturated or fully saturated, thereby including heteroaryl groups. Heterocyclic is also meant to include fused ring systems wherein one or more of the fused rings contain no heteroatoms. A heterocyclic group typically includes at least one atom selected from sulfur, nitrogen or oxygen. Examples of heterocyclic groups include, [1,3]dioxolane, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, quinoxalinyl, pyridazinonyl, tetrahydrofuryl and the like. Heterocyclic groups as used herein may optionally include further substitutent groups.

The terms “heteroaryl,” and “heteroaromatic,” as used herein, refer to a radical comprising a mono- or poly-cyclic aromatic ring, ring system or fused ring system wherein at least one of the rings is aromatic and includes a heteroatom. Heteroaryl is also meant to include fused ring systems including systems where one or more of the fused rings contain no heteroatoms. Heteroaryl groups typically include one ring atom selected from sulfur, nitrogen or oxygen. Examples of heteroaryl groups include, but are not limited to, pyridinyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, benzimidazolyl, benzooxazolyl, quinoxalinyl, and the like. Heteroaryl radicals can be attached to a parent molecule directly or through a linking moiety such as an aliphatic group or hetero atom. Heteroaryl groups as used herein may optionally include further substitutent groups.

The term “heteroarylalkyl,” as used herein, refers to a heteroaryl group as previously defined, attached to a parent molecule via an alkyl group. Examples include, but are not limited to, pyridinylmethyl, pyrimidinylethyl and the like. Heteroarylalkyl groups as used herein may optionally include further substitutent groups.

The term “acyl,” as used herein, refers to a radical formed by removal of a hydroxyl group from an organic acid and has the general formula —C(O)Rₐ where R is typically aliphatic, alicyclic or aromatic. Examples include aliphatic carbonyls, aromatic carbonyls, aliphatic sulfonyls, aromatic sulfinyls, aliphatic sulfinyls, aromatic phosphates, aliphatic phosphates and the like. Acyl groups as used herein may optionally include further substitutent groups.

The terms “substituent and substituent group,” as used herein, are meant to include groups that are typically added to other groups or parent compounds to enhance desired properties or give desired effects. Substituent groups can be protected or unprotected and can be added to one available site or to many available sites in a parent compound. Substituent groups may also be further substituted with other substituent groups and may be attached directly or via a linking group such as an alkyl or hydrocarbyl group to the parent compound. Such groups include without limitation, halogen, hydroxyl, alkyl, alkenyl, alkynyl, acyl (—C(O)Rₐ), carboxyl (—C(O)O—Rₐ), aliphatic, alicyclic, alkoxy, substituted oxo (—O—Rₐ), aryl, aralkyl, heterocyclic, heteroaryl, heteroarylalkyl, amino (—NR_{b}R_{c}), imino(═NR_{b}), amido (—C(O)NR_{b}R_{c} or —N(R_{b})C(O)Rₐ), azido (—N₃), nitro (—NO₂), cyano (—CN), carbamido (—OC(O)NR_{b}R_{c} or —N(R_{b})C(O)ORₐ), ureido (—N(R_{b})C(O)—NR_{b}R_{c}), thioureido (—N(R_{b})C(S)NR_{b}R_{c}), guanidinyl (—N(R_{b})C(═NR_{b})NR_{b}R_{c}), amidinyl (—C(═NR_{b})NR_{b}R_{c} or —N(R_{b})C(NR_{b})Rₐ), thiol (—SR_{b}), sulfinyl (—S(O)R_{b}), sulfonyl (—S(O)₂R_{b}) and sulfonamidyl (—S(O)₂NR_{b}R_{c} or —N(R_{b})S(O)₂R_{b}). Wherein each Rₐ, R_{b} and R_{c} is a further substituent group with a preferred list including without limitation alkyl, alkenyl, alkynyl, aliphatic, alkoxy, acyl, aryl, aralkyl, heteroaryl, alicyclic, heterocyclic and heteroarylalkyl.

The methods of the present invention illustrate the use of activated phosphite groups (e.g. compounds having activated phosphite containing substituent groups) in coupling reactions. As used herein, the term activated phosphorus groups include compounds, particularly monomer synthons for oligomer synthesis, that have an activated phosphite containing substituent group that is reactive with a free hydroxyl group to form a phosphorus-containing linkage. Such activated phosphite groups containing activated phosphorus atoms in the P^{III} valence state are known in the art and include, but are not limited to, phosphoramidite, H-phosphonate, phosphate triesters and phosphite containing chiral auxiliaries. A preferred synthetic solid phase synthesis utilizes phosphoramidites as activated phosphates. The phosphoramidites utilize P^{III} chemistry. The intermediate phosphite compounds are subsequently oxidized to the P^{V} state using known methods to yield, in a preferred embodiment, phosphodiester or phosphorothioate internucleotide linkages. Additional activated phosphates and phosphites are disclosed in Tetrahedron Report Number 309 (Beaucage and Iyer, Tetrahedron, 1992, 48, 2223-2311).

Activated phosphite groups are useful in the preparation of a wide range of oligomeric compounds including but not limited to oligonucleosides and oligonucleotides as well as oligonucleotides that have been modified or conjugated with other groups at the base or sugar or both. Also included are oligonucleotide mimetics including but not limited to peptide nucleic acids (PNA), morpholino nucleic acids, cyclohexenyl nucleic acids (CeNA), anhydrohexitol nucleic acids, locked nucleic acids (LNA and ENA), bicyclic and tricyclic nucleic acids, phosphonomonoester nucleic acids and cyclobutyl nucleic acids. A representative example of one type of oligomer synthesis that utilizes the coupling of an activated phosphorus group with a reactive hydroxyl group is the widely used phosphoramidite approach. A phosphoramidite synthon is reacted under appropriate conditions with a reactive hydroxyl group to form a phosphite linkage that is further oxidized to a phosphodiester or phosphorothioate linkage. This approach commonly utilizes nucleoside phosphoramidites of the formula:

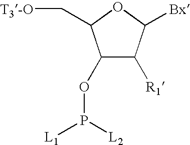

### Wherein

each Bx′ is an optionally protected heterocyclic base moiety;

each R₁′ is, independently, H, OH, or an optionally protected sugar substituent group;

T₃′ is H, a hydroxyl protecting group, a nucleoside, a nucleotide, an oligonucleoside or an oligonucleotide;

L₁ is N(R₁)R₂ or a group referred to below;

each R₂ and R₃ is, independently, C₁-C₁₀ straight or branched chain alkyl;

or R₂ and R₃ are joined together to form a 4- to 7-membered heterocyclic ring system including the nitrogen atom to which R₂ and R₃ are attached, wherein said ring system optionally includes at least one additional heteroatom selected from O, N and S;

L₂ is Pg-O—, Pg-S—, C₁-C₁₀ straight or branched chain alkyl, CH₃(CH₂)₀₋₁₀—O—, —NR₅R₆, or a group referred to below;

Pg is a protecting/blocking group; and

each R₅ and R₆ is, independently, hydrogen, C₁-C₁₀ straight or branched chain alkyl, cycloalkyl or aryl;

or optionally, R₅ and R₆, together with the nitrogen atom to which they are attached form a cyclic moiety that may include an additional heteroatom selected from O, S and N; or

L₁ and L₂ together with the phosphorus atom to which L₁ and L₂ are attached form a chiral auxiliary.

Groups that are attached to the phosphorus atom of internucleotide linkages before and after oxidation (L₁ and L₂) can include nitrogen containing cyclic moieties such as morpholine. Such oxidized internucleoside linkages include a phosphoromorpho-lidothioate linkage (Wilk et al., Nucleosides and nucleotides, 1991, 10, 319-322). Further cyclic moieties amenable to the present invention include mono-, bi- or tricyclic ring moieties which may be substituted with groups such as oxo, acyl, alkoxy, alkoxycarbonyl, alkyl, alkenyl, alkynyl, amino, amido, azido, aryl, heteroaryl, carboxylic acid, cyano, guanidino, halo, haloalkyl, haloalkoxy, hydrazino, ODMT, alkylsulfonyl, nitro, sulfide, sulfone, sulfonamide, thiol and thioalkoxy. A preferred bicyclic ring structure that includes nitrogen is phthalimido.

The term “protecting group,” as used herein, refers to a labile chemical moiety which is known in the art to protect reactive groups including without limitation, hydroxyl, amino and thiol groups, against undesired reactions during synthetic procedures. Protecting groups are typically used selectively and/or orthogonally to protect sites during reactions at other reactive sites and can then be removed to leave the unprotected group as is or available for further reactions. Protecting groups as known in the art are described generally in Greene and Wuts, Protective Groups in Organic Synthesis, 3rd edition, John Wiley & Sons, New York (1999).

Examples of hydroxyl protecting groups include, but are not limited to, benzyloxycarbonyl, 4-nitrobenzyloxycarbonyl, 4-bromobenzyloxycarbonyl, 4-methoxybenzyloxycarbonyl, methoxycarbonyl, tert-butoxycarbonyl (BOC), isopropoxycarbonyl, di-phenylmethoxycarbonyl, 2,2,2-trichloroethoxycarbonyl, 2-(trimethylsilyl)ethoxycarbonyl, 2-furfuryloxycarbonyl, allyloxycarbonyl (Alloc), acetyl (Ac), formyl, chloroacetyl, trifluoroacetyl, methoxyacetyl, phenoxyacetyl, benzoyl (Bz), methyl, t-butyl, 2,2,2-trichloroethyl, 2-trimethylsilyl ethyl, 1,1-dimethyl-2-propenyl, 3-methyl-3-butenyl, allyl, benzyl (Bn), para-methoxybenzyldiphenylmethyl, triphenylmethyl (trityl), 4,4′-dimethoxytriphenylmethyl (DMT), substituted or unsubstituted 9-(9-phenyl)xanthenyl (pixyl), tetrahydrofuryl, methoxymethyl, methylthiomethyl, benzyloxymethyl, 2,2,2-trichloroethoxymethyl, 2-(trimethylsilyl)ethoxymethyl, methanesulfonyl, para-toluene-sulfonyl, trimethylsilyl, triethylsilyl, triisopropylsilyl, and the like. Preferred hydroxyl protecting groups for the present invention are DMT and substituted or unsubstituted pixyl.

Examples of amino protecting groups include, but are not limited to, t-butoxy-carbonyl (BOC), 9-fluorenylmethoxycarbonyl (Fmoc), benzyloxycarbonyl, and the like.

Examples of thiol protecting groups include, but are not limited to, triphenylmethyl (Trt), benzyl (Bn), and the like.

The conjugated oligomeric compounds can be separated from a reaction mixture and further purified by various methods including but not limited to column chromatography, high pressure liquid chromatography, precipitation, or recrystallization. Further methods of synthesizing the conjugated oligomeric compounds of the formulae herein will be evident to those of ordinary skill in the art. Additionally, the various synthetic steps may be performed in an alternate sequence or order to give the desired compounds. Synthetic chemistry transformations and protecting group methodologies (protection and deprotection) useful in synthesizing the compounds described herein are known in the art and include, for example, those such as described in R. Larock, Comprehensive Organic Transformations, VCH Publishers (1989); T. W. Greene and P. G. M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

The conjugated oligomeric compounds described herein contain one or more asymmetric centers and thus give rise to enantiomers, diastereomers, and other stereoisomeric forms that may be defined, in terms of absolute stereochemistry, as (R)- or (S)-, or (D)- or (L)- for amino acids. The present invention is meant to include all such possible isomers, as well as their racemic and optically pure forms. Optical isomers may be prepared from their respective optically active precursors, or by resolution of racemic mixtures. The resolution of a racemic mixture can be carried out in the presence of a resolving agent, by chromatography or by repeated crystallization or by some combination of these techniques which are known to those skilled in the art. Further details regarding resolutions can be found in Jacques, et al., Enantiomers, Racemates, and Resolutions (John Wiley & Sons, 1981). When the compounds described herein contain olefinic double bonds, other unsaturation, or other centers of geometric asymmetry, and unless specified otherwise, it is intended that the compounds include both E and Z geometric isomers or cis- and trans-isomers. Likewise, all tautomeric forms are also intended to be included. The configuration of any carbon-carbon double bond appearing herein is selected for convenience only and is not intended to designate a particular configuration unless the text so states; thus a carbon-carbon double bond or carbon-heteroatom double bond depicted arbitrarily herein as trans may be cis, trans, or a mixture of the two in any proportion.

In one aspect of the present invention conjugated oligomeric compounds modulate gene expression by hybridizing to a nucleic acid target resulting in loss of its normal function. As used herein, the term “target nucleic acid” or “nucleic acid target” is used for convenience to encompass any nucleic acid capable of being targeted including without limitation DNA, RNA (including pre-mRNA and mRNA or portions thereof) transcribed from such DNA, and also cDNA derived from such RNA. In a preferred embodiment of the invention the target nucleic acid is a messenger RNA. In a further preferred embodiment the degradation of the targeted messenger RNA is facilitated by a RISC complex that is formed with oligomeric compounds of the invention. In another preferred embodiment the degradation of the targeted messenger RNA is facilitated by a nuclease such as RNaseH.

The hybridization of conjugated oligomeric compounds of the invention with their target nucleic acids is generally referred to as “antisense”. Consequently, the preferred mechanism in the practice of some preferred embodiments of the invention is referred to herein as “antisense inhibition.” Such antisense inhibition is typically based upon hydrogen bonding-based hybridization of oligonucleotide strands or segments such that at least one strand or segment is cleaved, degraded, or otherwise rendered inoperable. In this regard, it is presently preferred to target specific nucleic acid molecules and their functions for such antisense inhibition.

The functions of DNA to be interfered with can include replication and transcription. Replication and transcription, for example, can be from an endogenous cellular template, a vector, a plasmid construct or otherwise. The functions of RNA to be interfered with can include functions such as translocation of the RNA to a site of protein translation, translocation of the RNA to sites within the cell which are distant from the site of RNA synthesis, translation of protein from the RNA, splicing of the RNA to yield one or more RNA species, and catalytic activity or complex formation involving the RNA which may be engaged in or facilitated by the RNA. In the context of the present invention, “modulation” as applied to expression is meant to include either an increase (stimulation) or a decrease (inhibition) in the amount or levels of a nucleic acid molecule encoding the gene, e.g., DNA or RNA. Inhibition is often the preferred form of modulation of expression and mRNA is often a preferred target nucleic acid.

A commonly exploited antisense mechanism is RNase H-dependent degradation of the targeted RNA. RNase H is a ubiquitously expressed endonuclease that recognizes antisense DNA-RNA heteroduplexes, hydrolyzing the RNA strand. A further antisense mechanism involves the utilization of enzymes that catalyze the cleavage of RNA-RNA duplexes. These reactions are catalyzed by a class of RNAse enzymes including but not limited to RNAse III and RNAse L. The antisense mechanism known as RNA interference (RNAi) is operative on RNA-RNA hybrids and the like. Both RNase H-based antisense (usually using single-stranded compounds) and RNA interference (usually using double-stranded compounds known as siRNAs) are antisense mechanisms, typically resulting in loss of target RNA function.

Optimized siRNA and RNase H-dependent oligomeric compounds behave similarly in terms of potency, maximal effects, specificity and duration of action, and efficiency. Moreover it has been shown that in general, activity of dsRNA constructs correlated with the activity of RNase H-dependent single-stranded antisense oligomeric compounds targeted to the same site. One major exception is that RNase H-dependent antisense oligomeric compounds were generally active against target sites in pre-mRNA whereas siRNAs were not.

These data suggest that, in general, sites on the target RNA that were not active with RNase H-dependent oligonucleotides were similarly not good sites for siRNA. Conversely, a significant degree of correlation between active RNase H oligomeric compounds and siRNA was found, suggesting that if a site is available for hybridization to an RNase H oligomeric compound, then it is also available for hybridization and cleavage by the siRNA complex. Consequetly, once suitable target sites have been determined by either antisense approach, these sites can be used to design constructs that operate by the alternative antisense mechanism (Vickers et al., 2003, J. Biol. Chem. 278,7108). Moreover, once a site has been demonstrated as active for either an RNAi or an RNAse H oligomeric compound, a single-stranded RNAi oligomeric compound (ssRNAi or asRNA) can be designed.

In other embodiments of the present invention, single-stranded antisense oligomeric compounds are suitable. In some embodiments, the single-stranded oligomeric compounds may be “DNA-like”, in that the oligomeric compound has well characterized structural features, for example a plurality of unmodified H atoms at the 2′-positions or a stabilized backbone such as e.g., phosphorothioate, that is structurally suited for interaction with a target nucleic acid and recruitment and (activation) of RNase H.

The conjugated oligomeric compounds and associated methods of the present invention are also useful in the study, characterization, validation and modulation of small non-coding RNAs. These include, but are not limited to, microRNAs (miRNA), small nuclear RNAs (snRNA), small nucleolar RNAs (snoRNA), small temporal RNAs (stRNA) and tiny non-coding RNAs (tncRNA) or their precursors or processed transcripts or their association with other cellular components.

Small non-coding RNAs have been shown to function in various developmental and regulatory pathways in a wide range of organisms, including plants, nematodes and mammals. MicroRNAs are small non-coding RNAs that are processed from larger precursors by enzymatic cleavage and inhibit translation of mRNAs. stRNAs, while processed from precursors much like miRNAs, have been shown to be involved in developmental timing regulation. Other non-coding small RNAs are involved in events as diverse as cellular splicing of transcripts, translation, transport, and chromosome organization.

As modulators of small non-coding RNA function, the conjugated oligomeric compounds of the present invention find utility in the control and manipulation of cellular functions or processes such as regulation of splicing, chromosome packaging or methylation, control of developmental timing events, increase or decrease of target RNA expression levels depending on the timing of delivery into the specific biological pathway and translational or transcriptional control. In addition, the conjugated oligomeric compounds of the present invention can be further modified in order to optimize their effects in certain cellular compartments, such as the cytoplasm, nucleus, nucleolus or mitochondria.

The conjugated oligomeric compounds of the present invention can further be used to identify components of regulatory pathways of RNA processing or metabolism as well as in screening assays or devices.

The term “nucleoside,” as used herein, refers to a base-sugar combination. The base portion of the nucleoside is normally a heterocyclic base moiety. The two most common classes of such heterocyclic bases are purines and pyrimidines. Nucleotides are nucleosides that further include a phosphate group covalently linked to the sugar portion of the nucleoside. For those nucleosides that include a pentofuranosyl sugar, the phosphate group can be linked to either the 2′, 3′ or 5′ hydroxyl moiety of the sugar. Within the oligonucleotide structure, the phosphate groups are commonly referred to as forming the internucleoside linkages of the oligonucleotide, or in conjunction with the sugar ring, the backbone of the oligonucleotide. In forming oligonucleotides, the phosphate groups covalently link adjacent nucleosides to one another to form a linear polymeric compound. The normal internucleoside linkage of RNA and DNA is a 3′ to 5′ phosphodiester linkage.

In the context of this invention, the term “oligonucleoside” refers to a sequence of nucleosides that are joined by internucleoside linkages that do not have phosphorus atoms. Internucleoside linkages of this type are further described in the “modified internucleoside linkage” section below.

The term “oligonucleotide,” as used herein, refers to an oligomer or polymer of ribonucleic acid (RNA) or deoxyribonucleic acid (DNA) composed of naturally occurring nucleobases, sugars and phosphodiester internucleoside linkages.

The terms “oligomer” and “oligomeric compound,” as used herein, refer to a plurality of naturally occurring and/or non-naturally occurring nucleosides, joined together in a specific sequence, to form a polymeric structure. It is preferable that oligomeric compounds be capable of hybridizing a region of a target nucleic acid. Included in the terms “oligomer” and “oligomeric compound” are oligonucleotides, oligonucleotide analogs, oligonucleotide mimetics, oligonucleosides and chimeric combinations of these, and are thus intended to be broader than the term “oligonucleotide,” including all oligomers having all manner of modifications including but not limited to those known in the art. Oligomeric compounds are typically structurally distinguishable from, yet functionally interchangeable with, naturally-occurring or synthetic wild-type oligonucleotides. Thus, oligomeric compounds include all such structures that function effectively to mimic the structure and/or function of a desired RNA or DNA strand, for example, by hybridizing to a target. Such non-naturally occurring oligonucleotides are often desired over the naturally occurring forms because they often have enhanced properties, such as for example, enhanced cellular uptake, enhanced affinity for nucleic acid target and increased stability in the presence of nucleases.

Oligomeric compounds are typically prepared having enhanced properties compared to native oligonucleotides, against nucleic acid targets. A target is identified and an oligonucleotide is selected having an effective length and sequence that is complementary to a portion of the target sequence. Each nucleoside of the selected sequence is scrutinized for possible enhancing modifications. A preferred modification for one or more RNA like nucleosides would be the replacement of one or more of these RNA nucleosides with modified nucleosides that have the same 3′-endo conformational geometry. The modified nucleosides can enhance the chemical and nuclease stability of the parent oligomeric compound relative to the unmodified oligomer and may be much cheaper and easier to synthesize and/or incorporate into an oligonulceotide. The selected sequence can be further divided into regions and the nucleosides of each region evaluated for enhancing modifications that can provide an enhanced chimeric oligomeric compound such as blockmers, hemimers and gapmers (symmetric and asymmetric). Other chimeric oligomeric compounds that can used include positionally modified, alternating and fully modified motifs. Consideration is also given to the 5′- and 3′-termini as there are often advantageous modifications that can be made to one or more of the terminal nucleosides to enhance properties such as resistance to nucleases. Further modifications are also considered, such as modifying internucleoside linkages, addition of conjugate groups, addition of substituent groups to sugars or bases, replacing one or more nucleosides with nucleoside mimetics and other modifications that are known in the art that can enhance properties of the selected sequence for its intended target relative to an unmodified oligomer.

Oligomeric compounds are routinely prepared linearly but can be joined or otherwise prepared to be circular (by hybridization or by formation of a covalent bond) and may also include branching, however open linear structures are generally desired. In general, an oligomeric compound comprises a backbone of linked momeric subunits where each linked momeric subunit is directly or indirectly attached to a heterocyclic base moiety. Oligomeric compounds may also include monomeric subunits that are not linked to a heterocyclic base moiety thereby providing abasic sites. The linkages joining the monomeric subunits, the sugar moieties or surrogates and the heterocyclic base moieties can be independently modified giving rise to a plurality of motifs for the resulting oligomeric compounds such as blockmers, hemimers, gapmers and other chimeras.

Oligomeric compounds can include double-stranded constructs such as, for example, two oligomeric compounds forming a double stranded hybridized construct or a single strand with sufficient self complementarity to allow for hybridization and formation of a fully or partially double-stranded compound. In one embodiment of the invention, double-stranded oligomeric compounds encompass short interfering RNAs (siRNAs). As used herein, the term “siRNA” is defined as a double-stranded construct comprising a first and second strand and having a central complementary portion between the first and second strands and terminal portions that are optionally complementary between the first and second strands or with a target nucleic acid. Each strand in the complex may have a length as defined above and may further comprise a central complementary portion having one of these defined lengths. Each strand may further comprise a terminal unhybridized portion having from 1 to about 6 nucleobases in length. The siRNAs may also have no terminal portions (overhangs). The two strands of an siRNA can be linked internally leaving free 3′ or 5′ termini or can be linked to form a continuous hairpin structure or loop. The hairpin structure may contain an overhang on either the 5′ or 3′ terminus producing an extension of single-stranded character.

In one embodiment of the invention, double-stranded constructs are canonical siRNAs. As used herein, the term “canonical siRNA” is defined as a double-stranded oligomeric compound having a first strand and a second strand each strand being 21 nucleobases in length with the strands being complementary over 19 nucleobases and having on each 3′ termini of each strand a deoxy thymidine dimer (dTdT) which in the double-stranded compound acts as a 3′ overhang.

In another embodiment, the double-stranded constructs are blunt-ended siRNAs. As used herein the term “blunt-ended siRNA” is defined as an siRNA having no terminal overhangs. That is, at least one end of the double-stranded constructs is blunt. siRNAs whether canonical or blunt act to elicit dsRNAse enzymes and trigger the recruitment or activation of the RNAi antisense mechanism. In a further embodiment, single-stranded RNAi (ssRNAi) compounds that act via the RNAi antisense mechanism are contemplated. Further modifications can be made to the double-stranded compounds and may include conjugate groups attached to one of the termini, selected nucleobase positions, sugar positions or to one of the internucleoside linkages. Alternatively, the two strands can be linked via a non-nucleic acid moiety or linker group. When formed from only one strand, dsRNA can take the form of a self-complementary hairpin-type molecule that doubles back on itself to form a duplex. Thus, the dsRNAs can be fully or partially double-stranded. When formed from two strands, or a single strand that takes the form of a self-complementary hairpin-type molecule doubled back on itself to form a duplex, the two strands (or duplex-forming regions of a single strand) are complementary RNA strands that base pair in Watson-Crick fashion.

Further included in the present invention are oligomeric compounds including antisense oligonucleotides, external guide sequence (EGS) oligonucleotides, ribozymes, alternate splicers, primers, probes, and other oligomeric compounds which hybridize to at least a portion of the target nucleic acid. As such, these oligomeric compounds that are antisense to a nucleic acid target may be introduced in the form of single-stranded, double-stranded, circular or hairpin oligomeric compounds and may contain structural elements such as internal or terminal bulges, mismatches or loops. In general, nucleic acids (including oligonucleotides) may be described as “DNA-like” (i.e., having 2′-deoxy sugars and, generally, T rather than U bases) or “RNA-like” (i.e., having 2′-hydroxyl or 2′-modified sugars and, generally U rather than T bases). Once introduced to a system, the oligomeric compounds of the invention may elicit the action of one or more enzymes or structural proteins to effect modification of the target nucleic acid.

The oligomeric compounds in accordance with this invention comprise from about 8 to about 80 nucleobases (i.e. from about 8 to about 80 linked nucleosides). One of ordinary skill in the art will appreciate that this comprehends oligomeric compounds of 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleobases.

In one embodiment, the oligomeric compounds of the invention comprise 13 to 80 nucleobases. One having ordinary skill in the art will appreciate that this embodies oligomeric compounds of 13, 14, 15, 16, 17, 18, 19, 20, 21, 22, 23, 24, 25, 26, 27, 28, 29 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleobases.

In one embodiment, the oligomeric compounds of the invention comprise 13 to 50 nucleobases. One having ordinary skill in the art will appreciate that this embodies oligomeric compounds of 13, 14, 15, 16, 17, 18, 19, 20, 21, 22, 23, 24, 25, 26, 27, 28, 29 30, 31, 32, 33, 34, 35, 36, 37, 38, 39, 40, 41, 42, 43, 44, 45, 46, 47, 48, 49, or 50 nucleobases.

In one embodiment, the oligomeric compounds of the invention comprise 13 to 30 nucleobases. One having ordinary skill in the art will appreciate that this embodies oligomeric compounds of 13, 14, 15, 16, 17, 18, 19, 20, 21, 22, 23, 24, 25, 26, 27, 28, 29 or 30 nucleobases.

In one embodiment, the oligomeric compounds of the invention comprise 20 to 30 nucleobases. One having ordinary skill in the art will appreciate that this embodies oligomeric compounds of 20, 21, 22, 23, 24, 25, 26, 27, 28, 29, or 30 nucleobases.

In one embodiment, the oligomeric compounds of the invention comprise 15 to 25 nucleobases. One having ordinary skill in the art will appreciate that this embodies oligomeric compounds of 15, 16, 17, 18, 19, 20, 21, 22, 23, 24 or 25.

In one embodiment, the oligomeric compounds of the invention comprise 20 nucleobases.

In one embodiment, the oligomeric compounds of the invention comprise 19 nucleobases.

In one embodiment, the oligomeric compounds of the invention comprise 18 nucleobases.

In one embodiment, the oligomeric compounds of the invention comprise 17 nucleobases.

In one embodiment, the oligomeric compounds of the invention comprise 16 nucleobases.

In one embodiment, the oligomeric compounds of the invention comprise 15 nucleobases.

In one embodiment, the oligomeric compounds of the invention comprise 14 nucleobases.

In one embodiment, the oligomeric compounds of the invention comprise 13 nucleobases.

Oligomeric compounds can form double stranded structures by having a region of complementarity on one strand that forms a double stranded region or by hybridizing two oligomeric compounds having a degree of complementarity between the two to form a double stranded composition.

In one embodiment double stranded compositions comprise oligomeric compounds of 21 nucleobases in length with complementarity over 19 nucleobases and each having a 3′-dTdT dimer (deoxy thymidines) overhang.

In one embodiment double stranded compositions comprise oligomeric compounds of 15 to 25 nucleobases. One having ordinary skill in the art will appreciate that this embodies oligomeric compounds of 15, 16, 17, 18, 19, 20, 21, 22, 23, 24 or 25.

In one embodiment double stranded compositions comprise oligomeric compounds of 17 to 23 nucleobases. One having ordinary skill in the art will appreciate that this embodies oligomeric compounds of 17, 18, 19, 20, 21, 22 or 23 nucleobases.

In one embodiment double stranded compositions comprise oligomeric compounds of 19 to 21 nucleobases. One having ordinary skill in the art will appreciate that this embodies oligomeric compounds of 19, 20 or 21 nucleobases.

In one embodiment double stranded compositions comprise oligomeric compounds of 23 nucleobases each.

In one embodiment double stranded compositions comprise oligomeric compounds of 22 nucleobases each.

In one embodiment double stranded compositions comprise oligomeric compounds of 21 nucleobases each.

In one embodiment double stranded compositions comprise oligomeric compounds of 20 nucleobases each.

In one embodiment double stranded compositions comprise oligomeric compounds of 19 nucleobases each.

In one embodiment double stranded compositions comprise oligomeric compounds of 18 nucleobases each.

In one embodiment double stranded compositions comprise oligomeric compounds of 17 nucleobases each.

One having skill in the art armed with the oligomeric compounds illustrated herein will be able, without undue experimentation, to identify further oligomeric compounds.

Oligomeric compounds of the invention can comprise numerous chemical modifications that will enhance and or impart beneficial properties. One common method of chemical modification is to incorporate modified sugars at one or more positions within an oligomeric compound. The term “modified sugar,” as used herein, refers to modifications of native ribofuranose and deoxyribofuranose sugars used in the nucleosides and oligomeric compounds of the invention. Modified sugars comprise nucleosides where the heterocyclic base moiety or modified heterocyclic base moiety is typically maintained for hybridization with an appropriate target nucleic acid. Such “modified sugars” are often desired over the naturally occurring forms because of advantageous properties they can impart to an oligomeric compound such as, for example, enhanced cellular uptake, enhanced affinity for nucleic acid target and increased stability to nuclease degredation. The term “modified sugar” is intended to include all manner of modifications known in the art including without limitation modifications to ring atoms and/or addition of substituent groups.

In one aspect modified sugars include sugars comprising a substituent group. Such sugars can be referred to as a “substituted sugar” or “substituted sugar moiety. The substituent group can replace a hydrogen or other group thereby being an added substituent or a substituent that replaces a group such as the 2′-hydroxyl group of native RNA. Oligomeric compounds of the invention may contain one or more substituted sugar moieties and the substituent groups may vary from one nucleoside to another or may form regions or alternating motifs. These substituted sugar moieties may contain one, two, three, four or five substituents, at any position(s) on the sugar ring (namely 1′, 2′, 3′, 4′, or 5′). Oligomeric compounds are preferably modified at one or more positions including the 5′-position of the 5′-terminus, the 3′-position of the 3′-terminus, at any 2′-position of any nucleoside for 3′-5′-linked regions or at any 3′-position of any nucleoside for a 2′-5′-linked region. A more preferred substitution it the 2′-position of a 3′-5′-linked region.

The basic furanose ring system can be chemically manipulated in a number of different ways. The configuration of attachment of the heterocyclic base to the 1′-position can result in the α-anomer (down) or the β-anomer (up). The β-anomer is the anomer found in native DNA and RNA but both forms can be used to prepare oligomeric compounds. A further manipulation can be achieved through the substitution the native form of the furanose with the enantiomeric form e.g. replacement of a native D-furanose with its mirror image enantiomer, the L-furanose. Another way to manipulate the furanose ring system is to prepare stereoisomers such as for example substitution at the 2′-position to give either the ribofaranose (down) or the arabinofuranose (up) or substitution at the 3′-position to give the xylofuranose or by altering the 2′, and the 3′-position simultaneously to give a lyxofuranose. The use of stereoisomers of the same substituent can give rise to completely different conformational geometry such as for example 2′-F which is 3′-endo in the ribo configuration and 2′-endo in the arabino configuration.

One example of a non-ribofuranose sugar used to prepare oligomeric compounds is substitution of threose for ribose as shown below:

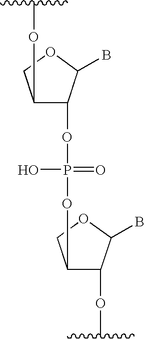

Initial interest in (3′,2′)-alpha-L-threose nucleic acid (TNA) was directed to the question of whether a DNA polymerase existed that would copy the TNA. It was found that certain DNA polymerases are able to copy limited stretches of a TNA template (reported in C&EN/Jan. 13, 2003). Further studies determined that TNA is capable of antiparallel Watson-Crick base pairing with complementary DNA, RNA and TNA oligonucleotides (Chaput et al, J. Am. Chem. Soc., 2003, 125, 856-857). When the (3′,2′)-alpha-L-threose nucleic acid was prepared and compared to the 2′ and 3′ amidate analogs (Wu et al., Organic Letters, 2002, 4(8), 1279-1282) the amidate analogs were shown to bind to RNA and DNA with comparable strength to that of RNA/DNA.

Suitable sugar substituent groups include, but are not limited to: hydroxyl, F, Cl, Br, SH, CN, OCN, CF₃, OCF₃, SOCH₃, SO₂CH₃, nitrate ester (ONO₂), NO₂, N₃, NH₂, O—, S—, or N(Rₖ)-alkyl; O—, S—, or N(Rₖ)-alkenyl; O—, S— or N(Rₖ)-alkynyl; O-alkylenyl-O-alkyl, alkynyl, alkaryl, aralkyl, O-alkaryl, O-aralkyl, a heterocyclic group linked to the sugar through an alkylenyl group, an aryl group further substituted with a heterocyclic group linked to the aryl group through an alkylenyl group, an amino group further substituted with an aminoalkylenyl group, polyalkylenylamino, substituted silyl, an RNA cleaving group, a reporter group, an intercalator, a group for improving the pharmacokinetic properties of an oligonucleotide, and a group for improving the pharmacodynamic properties of an oligonucleotide, wherein the substituent groups are optionally substituted with further substituent groups as previously defined.

Preferred sugar substituent groups are selected from: hydroxyl, F, O—, S—, or N(Rₖ)— alkyl; O—, S—, or N(Rₖ)-alkenyl; O—, S— or N(Rₖ)-alkynyl; or O-alkylene-O-alkyl, including O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙH]₂, where n and m are from 1 to about 10 and wherein the substituent groups are optionally substituted with further substituent groups as previously defined.

Preferred 2′-sugar substituent groups include F, methoxy (—O—CH₃), aminopropoxy (—O(CH₂)₃NIH₂), allyl (—CH₂—CH═CH₂), allyloxy (—O—CH₂—CH═CH₂), methoxyethoxy (—OCH₂CH₂OCH₃, also known as 2′-MOE) (Martin et al., Helv. Chim. Acta, 1995, 78, 486-504), dimethylaminooxyethoxy (—O(CH₂)₂ON(CH₃)₂ or DMAOE), and dimethyl-aminoethoxyethoxy (—O(CH₂)₂O(CH₂)₂N(CH₃)₂, also known as —O-dimethyl-aminoethoxyethyl or DMAEOE).

Oligonucleotides having the 2′-MOE side chain (Baker et al., J. Biol. Chem., 1997, 272, 11944-12000) demonstrate a very high binding affinity (greater than many similar 2′ modifications such as O-methyl, O-propyl, and O-aminopropyl), increased nuclease resistance, and have shown antisense inhibition of gene expression with promising features for in vivo use (Martin, Helv. Chim. Acta, 1995, 78, 486-504; Altmann et al., Chimia, 1996, 50, 168-176; Altmann et al., Biochem. Soc. Trans., 1996, 24, 630-637; and Altmann et al., Nucleosides and Nucleotides, 1997, 16, 917-926). Chimeric gapped oligonucleotides having 2′-MOE substituents in the wing nucleosides and an internal region of deoxyphosphorothioate nucleotides have shown effective reduction in the growth of tumors in animal models at low doses. 2′-MOE substituted oligonucleotides have also shown outstanding promise as antisense agents in several disease states. One such MOE substituted oligonucleotide Vitravene™ (Fomivirsen) has been approved for the treatment of cytomegalovirus (CMV)-induced retinitis in AIDS patients.

Further representative sugar substituents include groups of formula Ia or Ib:

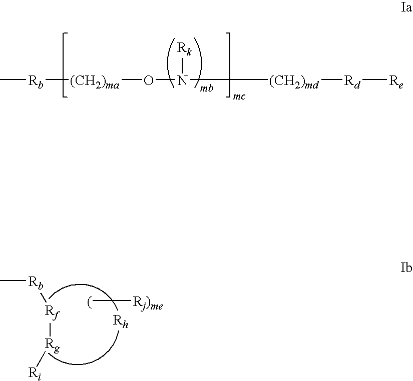

wherein:

R_{b} is O, S or NH;

R_{d} is a single bond linking (CH₂)_{md} to Rₑ, O, S or C(═O);

Rₑ is C₁-C₁₀ alkyl, N(Rₖ)(Rₘ), N(Rₖ)(Rₙ), N═C(Rₚ)(R_{q}), N═C(Rₚ)(Rᵣ) or has formula Ic;

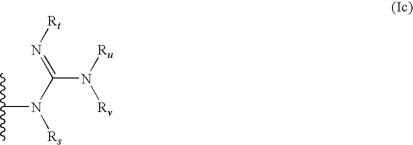

Rₚ and R_{q} are each independently hydrogen or C₁-C₁₀ alkyl;

Rᵣ is —Rₓ—R_{y};

each Rₛ, Rₜ, Rᵤ and Rᵥ is, independently, hydrogen, C(O)R_{w}, substituted or unsubstituted C₁-C₁₀ alkyl, substituted or unsubstituted C₂-C₁₀ alkenyl, substituted or unsubstituted C₂-C₁₀ alkynyl, alkylsulfonyl (alkyl-S(═O)(═O)—), arylsulfonyl, a chemical functional group or a conjugate group, wherein the substituent groups are selected from hydroxyl, amino, alkoxy, carboxy, benzyl, phenyl, nitro (—NO₂), thiol, thioalkoxy (—S-alkyl), halogen, alkyl, aryl, alkenyl and alkynyl;

or optionally, Rᵤ and Rᵥ, together form a phthalimido moiety with the nitrogen atom to which they are attached;

each R_{w} is, independently, substituted or unsubstituted C₁-C₁₀ alkyl, trifluoromethyl, cyanoethyloxy (—OCH₂CH₂CN), methoxy, ethoxy, t-butoxy, allyloxy, 9-fluorenylmethoxy, 2-(trimethylsilyl)-ethoxy, 2,2,2-trichloroethoxy, benzyloxy, butyryl, iso-butyryl, phenyl or aryl;

Rₖ is hydrogen, an amino protecting group or —Rₓ—R_{y};

Rₚ is hydrogen, an amino protecting group or —Rₓ—R_{y};

Rₓ is a bond or a linking moiety;

R_{y} is a chemical functional group, a conjugate group or a solid support medium;

each Rₘ and Rₙ is, independently, H, an amino protecting group, substituted or unsubstituted C₁-C₁₀ alkyl, substituted or unsubstituted C₂-C₁₀ alkenyl, substituted or unsubstituted C₂-C₁₀ alkynyl, wherein the substituent groups are selected from hydroxyl, amino, alkoxy, carboxy, benzyl, phenyl, nitro (—NO₂), thiol, thioalkoxy (—S-alkyl), halogen, alkyl, aryl, alkenyl, alkynyl; NH₃⁺, N(Rᵤ)(Rᵥ) guanidino and acyl where said acyl is an acid amide or an ester;

or Rₘ and Rₙ, together, are an amino protecting group, are joined in a ring structure that optionally includes an additional heteroatom selected from N and O or are a chemical functional group;

Rᵢ is OR_{z}, SR_{z}, or N(R_{z})₂;

each R_{z} is, independently, H, C₁-C₈ alkyl, C₁-C₈ haloalkyl, C(═NH)N(H)Rᵤ, C(═O)N(H)Rᵤ or OC(═O)N(H)Rᵤ;

R_{f}, R_{g} and Rₕ comprise a ring system having from about 4 to about 7 carbon atoms or having from about 3 to about 6 carbon atoms and 1 or 2 heteroatoms wherein said heteroatoms are selected from oxygen, nitrogen and sulfur and wherein said ring system is aliphatic, unsaturated aliphatic, aromatic, or saturated or unsaturated heterocyclic;

Rⱼ is alkyl or haloalkyl having 1 to about 10 carbon atoms, alkenyl having 2 to about 10 carbon atoms, alkynyl having 2 to about 10 carbon atoms, aryl having 6 to about 14 carbon atoms, N(Rₖ)(Rₘ) ORₖ, halo, SRₖ or CN;

ma is 1 to about 10;

each mb is, independently, 0 or 1;

mc is 0 or an integer from 1 to 10;

md is an integer from 1 to 10;

me is from 0, 1 or 2; and

provided that when mc is 0, md is greater than 1.

Representative substituent groups of Formula Ia, Formula Ib and Formula Ic are disclosed in U.S. patent application Ser. Nos. 09/130,973, 09/123,108, and 09/349,040 respectively. Representative acetamido (2′-O—CH₂C(O)N(Rₖ)(Rₙ)) substituent groups are disclosed in U.S. Pat. No. 6,147,200, and dimethylaminoethyloxyethyl substituent groups are disclosed in International Patent Application PCT/US99/17895.

Another 2′-substituent known to impart desirable properties (nuclease resistance, bioavailability, potency) to the parent oligomeric compound is the 2,4-dinitrophenyl (DNP) group. Poly[2′-O-(2,4-dinitrophenyl)]poly(A) [DNP-poly(A)] is a potent inhibitor for RNases A, B, S, T1, T2, and H, phosphodiesterases I and II (Rahman et. al., Anal. Chem. 1996, 68, 134-138) and reverse transcriptases, sang et. al., J. Biol. Chem. 1994, 269, 12024-12031). 2′-DNP substituted oligomers show good bioavailability as evidenced by DNP-poly[A] being spontaneously transported into isolated human lymphocytes and leukocytes, (Ashun, et. al., Antimicrobial Agents and Chemotherapy, 1996, 40 (10), 2311-2317). Increased potency was observed for Poly-DNP-siRNAs having lower IC₅₀ values and longer lasting growth inhibition than the corresponding unmodified siRNAs, against human cancer (lung adenocarcinoma A549) cells, (US Patent Application Publication US2004/0248841).

Some representative examples of substituted nucleosides amenable to the present invention include, but are not limited to those shown below:

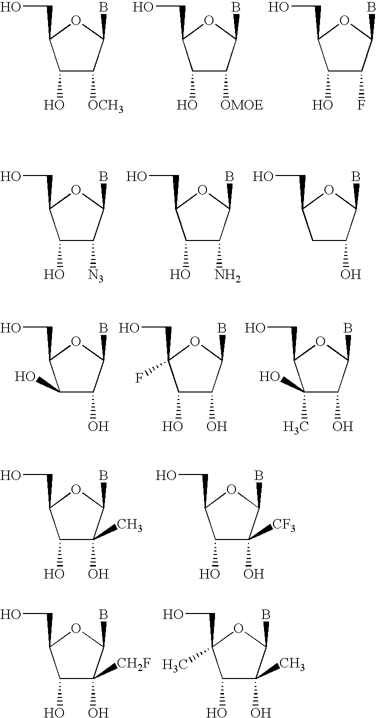

The terms “sugar mimetic” and “sugar surrogate,” as used herein, refer to non-furanosyl sugar substitutes that can mimic the native sugar when placed in an oligomeric compound and typically have one or more improved properties such as resistance to nuclease degredation or in combination with a non-native linking group may supply a neutral backbone. One of skill in the art can envisage many groups that can be interchanged with the native furanosyl group. Examples of sugar mimetics are shown below and are meant to be illustrative and not comprehensive.

Bicylco[3.1.0]hexane (methanocarba) nucleoside analogs, in which the furanose ring is replaced with a cylcopropane/cyclopentane bicyclic moiety can induce the 2′-exo or 3′-exo conformation, depending on structure, (Maier et al., Nucleic Acids Research. 2004, 32(12), 3642-3650). A 16-mer oligonucleotide, incorporating ten bicyclo[3.1.0]hexane pseudosugar rings fixed in a Northern conformation, resulted in an increase in Tm (Marquez et al., J. Med. Chem. 1996, 39, 3719-3747).

Oligomeric compounds have been prepared to include bicyclic and tricyclic sugar analogs (Steffens et al., Helv. Chim. Acta, 1997, 80, 2426-2439; Steffens et al., J. Am. Chem. Soc., 1999, 121, 3249-3255; and Renneberg et al., J. Am. Chem. Soc., 2002, 124, 5993-6002). The tricyclic analogs showed increased thermal stabilities (Tm's) when hybridized to DNA, RNA and itself, while the bicyclic analogs showed thermal stabilities approaching that of DNA duplexes.

Another oligonucleotide mimetic has been reported wherein the furanosyl ring has been replaced by a cyclobutyl moiety (see U.S. Pat. No. 3,539,044).

Representative U.S. patents that teach the preparation of such modified sugar structures include, but are not limited to, U.S. Pat. Nos. 4,981,957; 5,118,800; 5,319,080; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920.

Preferred sugar mimetics having bicyclic sugar moieties include “Locked Nucleic Acids” (LNAs) in which the 2′-hydroxyl group of the ribosyl sugar ring is linked to the 4′ carbon atom, thereby forming a 2′-C,4′-C-oxymethylene linkage to form a bicyclic sugar moiety (reviewed in Elayadi et al., Curr. Opinion Invens. Drugs, 2001, 2, 558-561; Braasch et al., Chem. Biol., 2001, 8, 1-7; and Orum et al., Curr. Opinion Mol. Ther., 2001, 3, 239-243; see also U.S. Pat. Nos. 6,268,490 and 6,670,461). The term locked nucleic acid has also been used in a broader sense in the literature to include any bicyclic structure that locks the sugar conformation. LNA's are commercially available from ProLigo (Paris, France and Boulder, Colo., USA).

The synthesis and preparation of the LNA monomers adenine, cytosine, guanine, 5-methyl-cytosine, thymine and uracil, along with their oligomerization, and nucleic acid recognition properties have been described (Koshkin et al., Tetrahedron, 1998, 54, 3607-3630 and WO 98/39352 and WO 99/14226).

Phosphorothioate-LNA, 2′-thio-LNA (Kumar et al., Bioorg. Med. Chem. Lett., 1998, 8, 2219-2222), and 2′-amino-LNA (Singh et al., J. Org. Chem., 1998, 63, 10035-10039) have also been prepared.

An isomer of LNA, is C-L-LNA which shows superior stability against a 3′-exo-nuclease (Frieden et al., Nucleic Acids Research, 2003, 21, 6365-6372), and when incorporated into antisense gapmers and chimeras showed potent antisense activity.

Preferred nucleosides having bicyclic sugar moieties also include ENA™ where an extra methylene group is added to the LNA bridge to give 2′-O,4′-ethylene-bridged nucleic acid ENA™, (Singh et al., Chem. Commun., 1998, 4, 455-456 and Morita et al., Bioorganic Medicinal Chemistry, 2003, 11, 2211-2226). ENA™'s have similar properties to LNA's showing enhanced affinity for DNA/RNA, high resistance to nuclease degradation and have been studied as antisense nucleic acids (see: Morita et al., Bioorg. Med. Chem., 2002, 12, 73-76; Morita et al., Bioorg. Med. Chem., 2003, 11, 2211-2226; Morita et al., Nucleic Acids Res. Suppl., 2002, Suppl. 2, 99-100; Morita et al., Nucleosides, Nucleotides & Nucleic Acids., 2003, 22, 1619-1621; and Takagi et al., Nucleic Acids Res. Supp., 2003, 3, 83-84). ENA™'s are commercially available from Sigma Genosys Japan.

A similar bicyclic sugar moiety that has been prepared and studied has the bridge going from the 3′-hydroxyl group via a single methylene group to the 4′ carbon atom of the sugar ring thereby forming a 3′-C,4′-C-oxymethylene linkage (3′,4′-BNA; see U.S. Pat. No. 6,043,060). The nitrogen containing analog (3′-amino-3′,4′-BNA) has also been prepared and shown to adopt a Southern type conformation (see Obika et al., Tetrahedron Lett., 2003, 44, 5267-5270). Another bicyclic sugar analog has the bridge going from the 2′-hydroxyl group via a single methylene group to the 1′-carbon atom of the sugar ring thereby forming a 2′-C,1′-C-oxymethylene linkage (1′,2′-oxetane; see Pushpangadan et al., J. Am. Chem. Soc., 2004, 126, 11484-11499).

These furanosyl sugar mimetics have the general structures shown below:

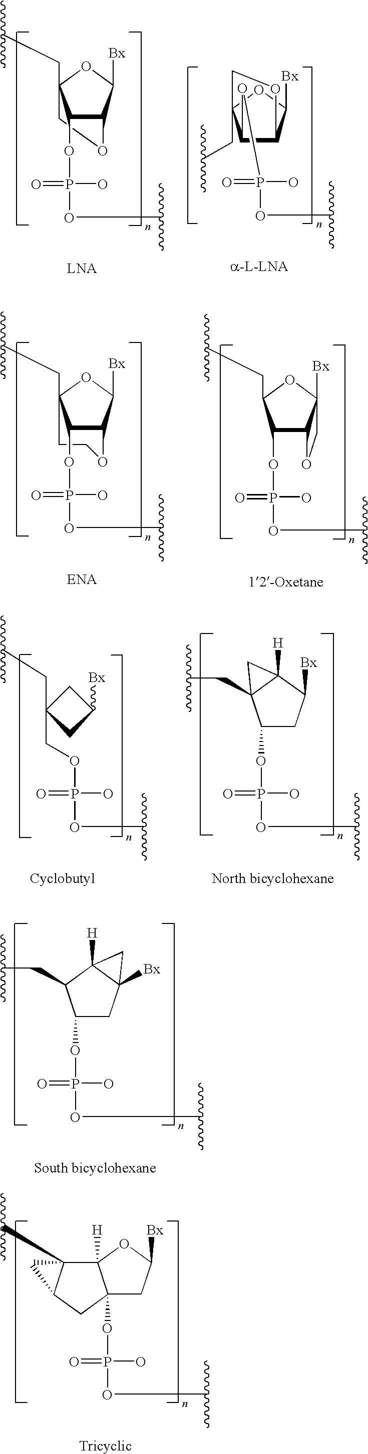

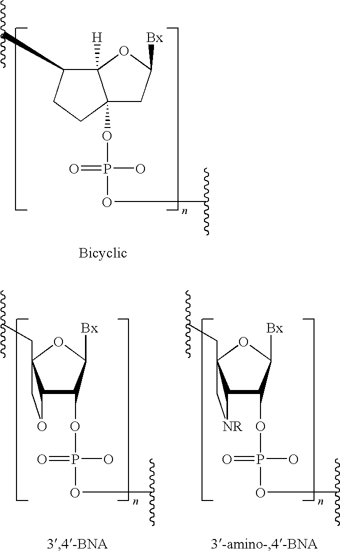

wherein

each Bx is, independently, a nucleobase or heterocyclic base moiety,

n is 1 when used as point modifiers at one or more locations whithin an oligomeric compound, from about 2 to about 6 when used as one or more regions within a chimeric oligomeric compound, and can be from 1 to about 50 when the oligomeric compound is prepared having uniformly modified sugar mimetics.

[symbol] represents an internucleotide linkage, a nucleoside, a nucleotide, an oligonucleotide, an oligonucleoside, H, a hydroxyl protecting group, a capping group, or an optionally linked conjugate group.

The sugar mimetics are shown above with phosphodiester groups but can be prepared having modified linkages such as for example phosphorothioate internucleoside linkages. The linkage group can be omitted for terminal nucleosides.

As used herein the term “heterocyclic base moiety” refers to nucleobases and modified or substitute nucleobases used to form nucleosides of the invention. The term “heterocyclic base moiety” includes unmodified nucleobases such as the native purine bases adenine (A) and guanine (G), and the pyrimidine bases thymine (T), cytosine (C) and uracil (U). The term is also intended to include all manner of modified or substitute nucleobases including but not limited to synthetic and natural nucleobases such as xanthine, hypoxanthine, 2-aminopyridine and 2-pyridone, 5-methylcytosine (5-me-C), 5-hydroxymethylenyl cytosine, 5-thiozolopyrimidine, 2-amino and 2-fluoroadenine, 2-propyl and other alkyl derivatives of adenine and guanine, 2-thio cytosine, uracil, thymine, 3-deaza guanine and adenine, 4-thiouracil, 5-uracil (pseudouracil), 5-propynyl (—C≡C—CH₃) uracil and cytosine and other alkynyl derivatives of pyrimidine bases, 5-halo particularly 5-bromo, 5-trifluoromethyl and other 5-substituted uracils and cytosines, 6-methyl and other alkyl derivatives of adenine and guanine, 6-azo uracil, cytosine and thymine, 7-methyl adenine and guanine, 7-deaza adenine and guanine, 8-halo, 8-amino, 8-aza, 8-thio, 8-thioalkyl, 8-hydroxyl and other 8-substituted adenines and guanines, universal bases, hydrophobic bases, promiscuous bases, size-expanded bases, and fluorinated bases as defined herein. Further modified nucleobases include tricyclic pyrimidines such as phenoxazine cytidine (1H-pyrimido[5,4-b][1,4]benzoxazin-2(3H)-one) and phenothiazine cytidine (1H-pyrimido[5,4-b][1,4]benzothiazin-2(3H)-one).

Further nucleobases (and nucleosides comprising the nucleobases) include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, those disclosed in Limbach et al., Nucleic. Acids Research, 1994, 22(12), 2183-2196, and those disclosed by Sanghvi, Y. S., Chapter 15, Antisense Research and Applications, pages 289-302, Crooke, S. T. and Lebleu, B., ed., CRC Press, 1993.

Certain of these nucleobases are particularly useful for increasing the binding affinity of the oligomeric compounds of the invention. These include 5-substituted pyrimidines, 6-azapyrimidines and N-2, N-6 and O-6 substituted purines, including 2-aminopropyl-adenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., eds., Antisense Research and Applications, CRC Press, Boca Raton, 1993, pp. 276-278) and are especially useful when combined with 2′-O-methoxyethyl (2′-MOE) sugar modifications.

Representative United States patents that teach the preparation of certain of the above noted modified nucleobases as well as other modified nucleobases include, but are not limited to, the above noted U.S. Pat. No. 3,687,808, as well as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,645,985; 5,830,653; 5,763,588; 6,005,096; 5,681,941, and 5,750,692.

The term “universal base” as used herein, refers to a moiety that may be substituted for any base. The universal base need not contribute to hybridization, but should not significantly detract from hybridization and typically refers to a monomer in a first sequence that can pair with a naturally occurring base, i.e A, C, G, T or U at a corresponding position in a second sequence of a duplex in which one or more of the following is true: (1) there is essentially no pairing (hybridization) between the two; or (2) the pairing between them occurs non-discriminantly with the universal base hybridizing one or more of the naturally occurring bases and without significant destabilization of the duplex. Exemplary universal bases include, without limitation, inosine, 5-nitroindole and 4-nitrobenzimidazole. For further examples and descriptions of universal bases see Survey and summary: the applications of universal DNA base analogs. Loakes, D. Nucleic Acids Research, 2001, 29, 12, 2437-2447.

The term “hydrophobic base” as used herein, refers to a heterocyclic base moiety that when used in a nucleoside monomer in a first sequence is able to pair with a naturally occurring base, i.e A, C, G, T or U at a corresponding position in a second sequence of a duplex in which one or more of the following is true: (1) the hydrophobic base acts as a non-polar close size and shape mimic (isostere) of one of the naturally occurring nucleosides; or (2) the hydrophobic base lacks all hydrogen bonding functionality on the Watson-Crick pairing edge.

The term “promiscuous base” as used herein, refers to a monomer in a first sequence that can pair with a naturally occurring base, i.e A, C, G, T or U at a corresponding position in a second sequence of a duplex in which the promiscuous base can pair non-discriminantly with more than one of the naturally occurring bases, i.e. A, C, G, T, U. Non-limiting examples of promiscuous bases are 6H,8H-3,4-dihydropyrimido-[4,5-c][1,2]oxazin-7-one and N⁶-methoxy-2,6-diaminopurine. For further information, see Polymerase recognition of synthetic oligodeoxyribonucleotides incorporating degenerate pyrimidine and purine bases. Hill, F.; Loakes, D.; Brown, D. M. Proc. Natl. Acad. Sci., 1998, 95, 4258-4263.

Other modified nucleobases include polycyclic heterocyclic moieties, which are routinely used in antisense applications to increase the binding properties of the modified strand to a target strand. The most studied modifications are targeted to guanosines hence they have been termed G-clamps or cytidine analogs.

Examples of G-clamps include substituted phenoxazine cytidine (e.g. 9-(2-aminoethoxy)-H-pyrimido[5,4-b][1,4]benzoxazin-2(3H)-one), carbazole cytidine (2H-pyrimido[4,5-b]indol-2-one) and pyridoindole cytidine (H-pyrido[3′,2′:4,5]pyrrolo[2,3-d]pyrimidin-2-one).

Representative cytosine analogs that make 3 hydrogen bonds with a guanosine in a second oligonucleotide include 1,3-diazaphenoxazine-2-one (Kurchavov, et al., Nucleosides and Nucleotides, 1997, 16, 1837-1846), 1,3-diazaphenothiazine-2-one (Lin, K.-Y.; Jones, R. J.; Matteucci, M. J. Am. Chem. Soc. 1995, 117, 3873-3874) and 6,7,8,9-tetrafluoro-1,3-diazaphenoxazine-2-one (Wang, J.; Lin, K.-Y., Matteucci, M. Tetrahedron Lett. 1998, 39, 8385-8388). When incorporated into oligonucleotides these base modifications hybridized with complementary guanine (the latter also hybridized with adenine) and enhanced helical thermal stability by extended stacking interactions (see U.S. patent application Ser. No. 10/013,295).

Further tricyclic, tetracyclic heteroaryl and polycyclic heterocyclic base moieties amenable to the present invention are disclosed in U.S. Pat. Nos. 5,434,257; 5,502,177; 5,646, 269; 6,028,183, and 6,007,992, and U.S. patent application Ser. No. 09/996,292.

The enhanced binding affinity of these derivatives together with their uncompromised sequence specificity makes them valuable heterocyclic base moieties for the development of more potent antisense-based drugs. In vitro experiments demonstrated that heptanucleotides containing phenoxazine substitutions are able to activate RNaseH, enhance cellular uptake and increase antisense activity (Lin, K.-Y.; Matteucci, M. J. Am. Chem. Soc. 1998, 120, 8531-8532). The activity enhancement was even more pronounced for a single G-clamp substitution, which significantly improved the in vitro potency of a 20-mer 2′-deoxyphosphorothioate oligonucleotide (Flanagan, W. M.; Wolf, J. J.; Olson, P.; Grant, D.; Lin, K.-Y.; Wagner, R. W.; Matteucci, M. Proc. Natl. Acad. Sci. USA, 1999, 96, 3513-3518).

The “term internucleoside linking group” as used herein is meant to include all manner of groups used to link monomer synthons in oligomer synthesis. The terms “modified internucleoside linkage” and “modified backbone,” or simply “modified linkage” as used herein, refer to modifications of the phosphodiester internucleoside linkage between two adjacent monomers such as nucleosides in an oligomeric compound. Modifications include but are not limited to substitution of atoms around the phosphate group or replacement of the phosphodiester linkage with a non-phosphorus internucleoside linkage.

Internucleoside linkages containing a phosphorus atom therein include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Oligonucleotides having inverted polarity can comprise a single 3′ to 3′ linkage at the 3′-most internucleotide linkage i.e. a single inverted nucleoside residue which may be abasic (the nucleobase is missing or has a hydroxyl group in place thereof). Various salts, mixed salts and free acid forms are also included. Representative U.S. patents that teach the preparation of the above phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050.

In the C. elegans system, modification of the internucleotide linkage (phosphorothioate in place of phosphodiester) did not significantly interfere with RNAi activity, indicating that oligomeric compounds of the invention can have one or more modified internucleoside linkages, and retain activity. Indeed, such modified internucleoside linkages are often desired over the naturally occurring phosphodiester linkage because of advantageous properties they can impart such as, for example, enhanced cellular uptake, enhanced affinity for nucleic acid target and increased stability in the presence of nucleases.

Another phosphorus containing modified internucleoside linkage is the phosphono-monoester (see U.S. Pat. Nos. 5,874,553 and 6,127,346). Phosphonomonoester nucleic acids have useful physical, biological and pharmacological properties in the areas of inhibiting gene expression (antisense oligonucleotides, ribozymes, sense oligonucleotides and triplex-forming oligonucleotides), as probes for the detection of nucleic acids and as auxiliaries for use in molecular biology.

As previously defined an oligonucleoside refers to a sequence of nucleosides that are joined by internucleoside linkages that do not have phosphorus atoms. Non-phosphorus containing internucleoside linkages include short chain alkyl, cycloalkyl, mixed heteroatom alkyl, mixed heteroatom cycloalkyl, one or more short chain heteroatomic and one or more short chain heterocyclic. These internucleoside linkages include but are not limited to siloxane, sulfide, sulfoxide, sulfone, acetyl, formacetyl, thioformacetyl, methylene formacetyl, thioformacetyl, alkeneyl, sulfamate; methyleneimino, methylenehydrazino, sulfonate, sulfonamide, amide and others having mixed N, O, S and CH₂ component parts. Representative United States patents that teach the preparation of the above oligonucleosides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439.

Some additional examples of modified internucleoside linkages that do not contain a phosphorus atom therein include, —CH₂—NH—O—CH₂—, —CH₂—N(CH₃)—O—CH₂— (known as a methylene (methylimino) or MMI backbone), —CH₂—O—N(CH₃)—CH₂—, —CH₂—N(CH₃)—N(CH₃)—CH₂— and —O—N(CH₃)—CH₂—CH₂— (wherein the native phosphodiester internucleotide linkage is represented as —O—P(═O)(OH)—O—CH₂—). The MMI type and amide internucleoside linkages are disclosed in the below referenced U.S. Pat. Nos. 5,489,677 and 5,602,240, respectively.

The terms “oligomer mimetic” and “oligonucleotide mimetic,” as used herein, refer to oligomeric compounds wherein the fliranose ring and the internucleoside linkage of the subunits are replaced with novel groups. The heterocyclic base moieties in the resulting oligonucleotide mimetic can hybridize to a nucleic acid target as would a native oligonucleotide of the same sequence but the modified sugar and linkage provides advantageous properties including but not limited to enhanced cellular uptake, enhanced affinity for nucleic acid target and increased stability in the presence of nucleases. Some non-limiting examples of “oligomer mimetics” are given below.

Replacing the sugar-backbone of an oligonucleotide with an amide containing backbone, provides peptide nucleic acids (PNA). The first PNA's reported (Nielsen et al., Science, 1991, 254, 1497-1500) consisted of nucleobases linked to the aza nitrogen atoms of the amide portion of an aminoethylglycine (aeg) backbone. These mimetics displayed favorable hybridization properties, high biological stability and are electrostatically neutral molecules. In one recent study PNA's were used to correct aberrant splicing in a transgenic mouse model (Sazani et al., Nat. Biotechnol., 2002, 20, 1228-1233). Since the first reports, numerous modifications have since been made to the basic PNA backbone, for example, incorporating a constrained cyclic aminoethylpropyl (aep) group, in place of the aeg group. Representative United States patents that teach the preparation of PNA oligomeric compounds include, but are not limited to, U.S. Pat. Nos. 5,539,082; 5,714,331; and 5,719,262. PNA's can be obtained commercially from Applied Biosystems (Foster City, Calif., USA).

Another class of oligonucleotide mimetic is based on nucleobases attached to linked morpholino units to form morpholino nucleic acid. A number of linking groups have been reported that link the morpholino monomeric units in a morpholino nucleic acid. A preferred class of linking groups has been selected to give a non-ionic oligomeric compound, which are less likely to have undesired interactions with cellular proteins, (Dwaine A. Braasch and David R. Corey, Biochemistry, 2002, 41(14), 4503-4510). Morpholino-based oligomeric compounds are disclosed in U.S. Pat. Nos. 5,034,506. 5,166,315, and 5,185,444 and several studies on them have been reported (see: Genesis, volume 30, issue 3, 2001 and Heasman, J., Dev. Biol., 2002, 243, 209-214, and Nasevicius et al., Nat. Genet., 2000, 26, 216-220; and Lacerra et al., Proc. Natl. Acad. Sci., 2000, 97, 9591-9596).

A further class of oligonucleotide mimetic is cyclohexenyl nucleic acids (CeNA), whereby the sugar-backbone is replaced with a cyclohexenyl ring. CeNA DMT protected phosphoramidite monomers have been prepared and used for oligomeric synthesis using standard phosphoramidite chemistry. Fully modified cyclohexenyl nucleic acids and oligonucleotides having specific positions modified with CeNA have been prepared and studied (see Wang et al., J. Am. Chem. Soc., 2000, 122, 8595-8602). In general, the incorporation of CeNA monomers into a DNA chain increases its stability in DNA/RNA hybrids, and was shown by NMR and circular dichroism to proceed with easy conformational adaptation. CeNA oligoadenylates formed complexes with RNA and DNA complements with similar stability to the native complexes. Furthermore, a sequence targeting RNA that incorporated CeNA, was stable to serum and able to activate E. Coli RNase resulting in cleavage of the target RNA strand.

Another class of oligonucleotide mimetic (anhydrohexitol nucleic acid) can be prepared from one or more anhydrohexitol nucleosides (see, Wouters and Herdewijn, Bioorg. Med. Chem. Lett., 1999, 9, 1563-1566). The above oligonucleotide mimetics can be considered as repeating units of the monomers depicted below:

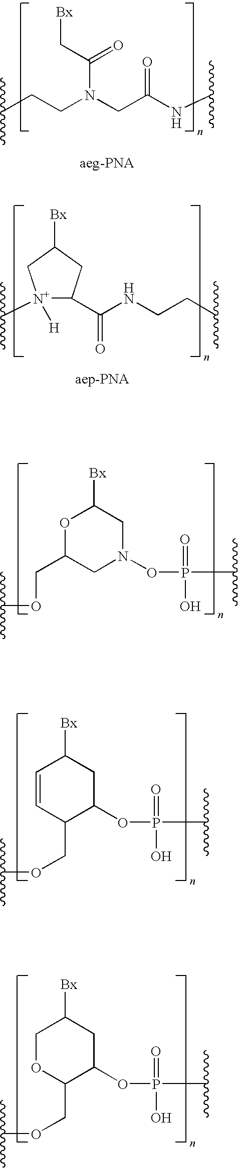

morpholino nucleic acid cyclohexenyl nucleic acid anhydrohexitol nucleic acid
(MF) (CeNA)

wherein,

each Bx is independently a nucleobase,

n is from 2 to about 50, and

[symbol] the squigly line represents connection to the next repeating monomer, or end terminus.

It is not necessary for all positions in an oligomeric compound to be uniformly modified, and in fact more than one of the modifications described herein may be incorporated in a single oligomeric compound or even at a single monomeric subunit such as a nucleoside within an oligomeric compound. The present invention also includes oligomeric compounds which are chimeric oligomeric compounds. “Chimeric” oligomeric compounds or “chimeras,” in the context of this invention, are oligomeric compounds which contain two or more chemically distinct regions, each made up of at least one monomer unit, i.e., a nucleotide in the case of a nucleic acid based oligomer.

Chimeric oligomeric compounds typically contain at least one region modified so as to confer increased resistance to nuclease degradation, increased cellular uptake, and/or increased binding affinity for the target nucleic acid. Chimeric oligomeric compounds are also being used in double stranded compositions wherein each strand is modified chimerically to have properties that will enhance its particular activity. An additional region of the oligomeric compound may serve as a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of inhibition of gene expression. Consequently, comparable results can often be obtained with shorter oligomeric compounds when chimeras are used, compared to for example unmodified or phosphorothioate deoxyoligonucleotides hybridizing to the same target region. Cleavage of the RNA target can be routinely detected by gel electrophoresis and, if necessary, associated nucleic acid hybridization techniques known in the art.

Chimeric oligomeric compounds of the invention may be formed as composite structures of two or more oligonucleotides, oligonucleotide analogs, oligonucleosides and/or oligonucleotide mimetics as described above. Routinely used chimeric compounds include but are not limited motifs selected from hemimer, blockmer, gapmer, alternating, fully modified or positional motifs. The modified nucleosides or nucleoside mimics that make up the point modifications or regional modifications that define a chimeric oligomeric compound include without limitation native or modified DNA and RNA, locked nucleic acids (LNA, which encompasses ENA™ as described below), peptide nucleic acids (PNA), morpholinos, and others described herein.

Representative United States patents that teach the preparation of such hybrid structures include, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, each of which is herein incorporated by reference in its entirety.

Oligomerization of modified and unmodified nucleosides is performed according to literature procedures for oligonucleotide synthesis (Protocols for Oligonucleotides and Analogs, Ed. Agrawal (1993), Humana Press). Additional methods for solid-phase synthesis may be found in Caruthers U.S. Pat. Nos. 4,415,732; 4,458,066; 4,500,707; 4,668,777; 4,973,679; and 5,132,418; and Koster U.S. Pat. Nos. 4,725,677 and Re. 34,069. In addition specific protocols for the synthesis of oligomeric compounds of the invention are illustrated in the examples below.

Oligonucleotides are generally prepared either in solution or on a support medium, e.g. a solid support medium. In general a first synthon (e.g. a monomer, such as a nucleoside) is first attached to a support medium, and the oligonucleotide is then synthesized by sequentially coupling monomers to the support-bound synthon. This iterative elongation eventually results in a final oligomeric compound or other polymer such as a polypeptide. Suitable support medium can be soluble or insoluble, or may possess variable solubility in different solvents to allow the growing support bound polymer to be either in or out of solution as desired. Traditional support medium such as solid support media are for the most part insoluble and are routinely placed in reaction vessels while reagents and solvents react with and/or wash the growing chain until the oligomer has reached the target length, after which it is cleaved from the support and, if necessary further worked up to produce the final polymeric compound. More recent approaches have introduced soluble supports including soluble polymer supports to allow precipitating and dissolving the iteratively synthesized product at desired points in the synthesis (Gravert et al., Chem. Rev., 1997, 97, 489-510).

Commercially available equipment routinely used for the support medium based synthesis of oligomeric compounds and related compounds is sold by several vendors including, for example, Applied Biosystems (Foster City, Calif.). Any other means for such synthesis known in the art may additionally or alternatively be employed. Suitable solid phase techniques, including automated synthesis techniques, are described in F. Eckstein (ed.), Oligonucleotides and Analogues, a Practical Approach, Oxford University Press, New York (1991).

RNA and RNA analogs are synthesized much the same as DNA and DNA analogs are synthesized with the primary difference being that the 2′-OH group needs to be protected. The synthesis of RNA can be carried out following literature procedures (Scaringe, Methods (2001), 23, 206-217; Gait et al., Applications of Chemically synthesized RNA in RNA:Protein Interactions, Ed. Smith (1998), 1-36; and Gallo et al., Tetrahedron (2001), 57, 5707-5713). A number of orthogonal protecting schemes have been published for the synthesis of RNA. A current list of some of the major companies currently offering RNA products include Pierce Nucleic Acid Technologies, Dharmacon Research Inc., Ameri Biotechnologies Inc., and Integrated DNA Technologies, Inc. One company, Princeton Separations, is marketing an RNA synthesis activator advertised to reduce coupling times especially with TOM and TBDMS chemistries. Such an activator would also be amenable to the present invention. The primary groups being used for commercial RNA synthesis are:
TBDMS=5′-O-DMT-2′-O-t-butyldimethylsilyl;
TOM=2′-O-[(triisopropylsilyl)oxy]methyl;
DOD/ACE=5′-O-bis(trimethylsiloxy)cyclododecyloxysilylether-2′-O-bis(2-acetoxyethoxy)methyl;
FPMP=5′-O-DMT-2′-O-[1(2-fluorophenyl)-4-methoxypiperidin-4-yl].

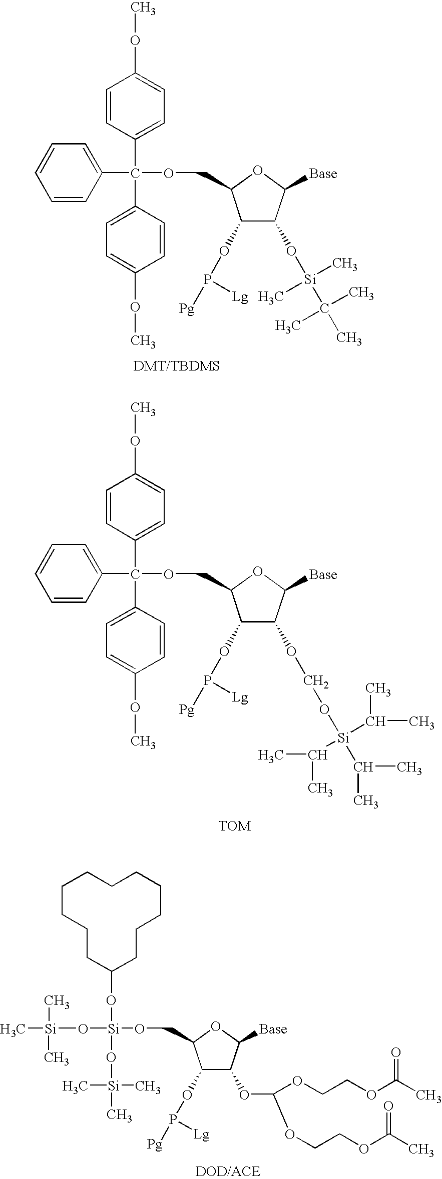

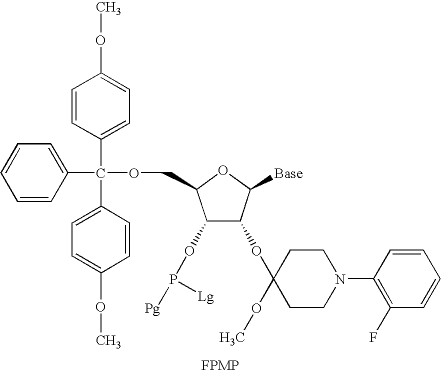

All of the aforementioned RNA synthesis strategies are amenable to the present invention. Strategies that would be a hybrid of the above e.g. using a 5′-protecting group from one strategy with a 2′-O-protecting from another strategy is also amenable to the present invention.

The term support medium is intended to include all forms of support known to one of ordinary skill in the art for the synthesis of oligomeric compounds and related compounds such as peptides. Some representative support medium that are amenable to the methods of the present invention include but are not limited to the following: controlled pore glass (CPG); oxalyl-controlled pore glass (see, e.g., Alul, et al., Nucleic Acids Research 1991, 19, 1527); silica-containing particles, such as porous glass beads and silica gel such as that formed by the reaction of trichloro-[3-(4-chloromethyl)-phenyl]propylsilane and porous glass beads (see Parr and Grohmann, Angew. Chem. Internal. Ed. 1972, 11, 314, sold under the trademark “PORASIL E” by Waters Associates, Framingham, Mass., USA); the mono ester of 1,4-dihydroxymethyl-enylbenzene and silica (see Bayer and Jung, Tetrahedron Lett., 1970, 4503, sold under the trademark “BIOPAK” by Waters Associates); TENTAGEL (see, e.g., Wright, et al., Tetrahedron Letters 1993, 34, 3373); cross-linked styrene/divinylbenzene copolymer beaded matrix or POROS, a copolymer of polystyrene/divinylbenzene (available from Perceptive Biosystems); soluble support medium, polyethylene glycol PEG's (see Bonora et al., Organic Process Research & Development, 2000, 4, 225-231).

Further support medium amenable to the present invention include without limitation PEPS support a polyethylene (PE) film with pendant long-chain polystyrene (PS) grafts (molecular weight on the order of 10⁶, (see Berg, et al., J. Am. Chem. Soc., 1989, 111, 8024 and International Patent Application WO 90/02749)). The loading capacity of the film is as high as that of a beaded matrix with the additional flexibility to accommodate multiple syntheses simultaneously. The PEPS film may be fashioned in the form of discrete, labeled sheets, each serving as an individual compartment. During all the identical steps of the synthetic cycles, the sheets are kept together in a single reaction vessel to permit concurrent preparation of a multitude of peptides at a rate close to that of a single peptide by conventional methods. Also, experiments with other geometries of the PEPS polymer such as, for example, non-woven felt, knitted net, sticks or microwellplates have not indicated any limitations of the synthetic efficacy.

Further support medium amenable to the present invention include without limitation particles based upon copolymers of dimethylacrylamide cross-linked with N,N′-bisacryloylethylenediamine, including a known amount of N-tertbutoxycarbonyl-beta-alanyl-N′-acryloylhexamethylenediamine. Several spacer molecules are typically added via the beta alanyl group, followed thereafter by the amino acid residue subunits. Also, the beta alanyl-containing monomer can be replaced with an acryloyl safcosine monomer during polymerization to form resin beads. The polymerization is followed by reaction of the beads with ethylenediamine to form resin particles that contain primary amines as the covalently linked functionality. The polyacrylamide-based supports are relatively more hydrophilic than are the polystyrene-based supports and are usually used with polar aprotic solvents including dimethylformamide, dimethylacetamide, N-methylpyrrolidone and the like (see Atherton, et al., J. Am. Chem. Soc., 1975, 97, 6584, Bioorg. Chem., 1979, 8, 351, and J. C. S. Perkin I 538 (1981)).

Further support medium amenable to the present invention include without limitation a composite of a resin and another material that is also substantially inert to the organic synthesis reaction conditions employed. One exemplary composite (see Scott, et al., J. Chrom. Sci., 1971, 9, 577) utilizes glass particles coated with a hydrophobic, cross-linked styrene polymer containing reactive chloromethyl groups, and is supplied by Northgate Laboratories, Inc., of Hamden, Conn., USA. Another exemplary composite contains a core of fluorinated ethylene polymer onto which has been grafted polystyrene (see Kent and Merrifield, Israel J. Chem. 1978, 17, 243 and van Rietschoten in Peptides 1974, Y. Wolman, Ed., Wiley and Sons, New York, 1975, pp. 113-116). Contiguous solid support media other than PEPS include without limitation cotton sheets (Lebl and Eichler, Peptide Res. 1989, 2, 232) and hydroxypropylacrylate-coated polypropylene membranes (Daniels, et al., Tetrahedron Lett. 1989, 4345). Acrylic acid-grafted polyethylene-rods and 96-microtiter wells are generally used to immobilize the growing peptide chains and to perform the compartmentalized synthesis. (Geysen, et al., Proc. Natl. Acad. Sci. USA, 1984, 81, 3998). A “tea bag” containing traditionally-used polymer beads. (Houghten, Proc. Natl. Acad. Sci. USA, 1985, 82, 5131). Simultaneous use of two different supports with different densities (Tregear, Chemistry and Biology of Peptides, J. Meienhofer, ed., Ann Arbor Sci. Publ., Ann Arbor, 1972 pp. 175-178). Combining of reaction vessels via a manifold (Gorman, Anal. Biochem., 1984, 136, 397). Multicolnum solid-phase synthesis (e.g., Krchnak, et al., Int. J. Peptide Protein Res., 1989, 33, 209), and Holm and Meldal, in “Proceedings of the 20th European Peptide Symposium”, G. Jung and E. Bayer, eds., Walter de Gruyter & Co., Berlin, 1989 pp. 208-210). Cellulose paper (Eichler, et al., Collect. Czech. Chem. Commun., 1989, 54, 1746). Support mediumted synthesis of peptides have also been reported (see, Synthetic Peptides: A User's Guide, Gregory A. Grant, Ed. Oxford University Press 1992; U.S. Pat. Nos. 4,415,732; 4,458,066; 4,500,707; 4,668,777; 4,973,679; 5,132,418; 4,725,677 and Re-34,069.)

Capping reagents are routinely used in oliogmeric compound synthesis to block reactive sites on compounds as well as on support media. Capping reagents are meant to include all manner of reagents used in oligomer synthesis including without mixtures of Cap A and Cap B. Representative mixtures include: Cap A: acetic anhydride in acetonitrile or tetrahydrofuran; chloroacetic anhydride in acetonitrile or tetrahydrofuran; Cap B: N-methylimidazole and pyridine in acetonitrile or tetrahydrofuran; 4-dimethylaminopyridine (DMAP) and pyridine in acetonitrile or tetrahydrofuran; 2,6-lutidine and N-methylimidazole in acetonitrile or tetrahydrofuran. A more detailed description capping reagents is discussed in U.S. Pat. No. 4,816,571, issued Mar. 28, 1989 which is incorporated herein by reference. A preferred capping reagent is acetic anhydride routinely used as a mixture of cap A and cap B.

In the context of this invention, “hybridization” means hydrogen bonding, which may be Watson-Crick, Hoogsteen or reversed Hoogsteen hydrogen bonding, between the heterocyclic base moieties of complementary nucleosides. For example, adenine and thymine are complementary nucleobases which pair through the formation of hydrogen bonds. “Complementary,” as used herein, refers to the capacity for precise pairing between two nucleotides. For example, if a nucleotide at a certain position of an oligonucleotide is capable of hydrogen bonding with a nucleotide at the same position of a DNA or RNA molecule, then the oligonucleotide and the DNA or RNA are considered to be complementary to each other at that position. The oligonucleotide and the DNA or RNA are complementary to each other when a sufficient number of corresponding positions in each molecule are occupied by nucleotides which can hydrogen bond with each other. Thus, “specifically hybridizable” and “complementary” are terms which are used to indicate a sufficient degree of complementarity or precise pairing such that stable and specific binding occurs between the oligonucleotide and the DNA or RNA target. It is understood in the art that the sequence of an antisense oligomeric compound need not be 100% complementary to that of its target nucleic acid to be specifically hybridizable. An antisense oligomeric compound is specifically hybridizable when binding of the compound to the target DNA or RNA molecule interferes with the normal function of the target DNA or RNA to cause a complete or partial loss of function, and there is a sufficient degree of complementarity to avoid non-specific binding of the antisense oligomeric compound to non-target sequences under conditions in which specific binding is desired, i.e., under physiological conditions in the case of therapeutic treatment, or under conditions in which in vitro or in vivo assays are performed. Moreover, an oligonucleotide may hybridize over one or more segments such that intervening or adjacent segments are not involved in the hybridization event (e.g., a loop structure, mismatch or hairpin structure).

The oligomeric compounds of the present invention comprise at least 70%, at least 75%, at least 80%, at least 85%, at least 90%, at least 95%, at least 99%, or 100% sequence complementarity to a target region within the target nucleic acid sequence to which they are targeted. For example, an antisense oligomeric compound in which 18 of 20 nucleobases of the antisense oligomeric compound are complementary to a target region, and would therefore specifically hybridize, would represent 90 percent complementarity. In this example, the remaining noncomplementary nucleobases may be clustered or interspersed with complementary nucleobases and need not be contiguous to each other or to complementary nucleobases. As such, an antisense oligomeric compound which is 18 nucleobases in length having 4 (four) noncomplementary nucleobases which are flanked by two regions of complete complementarity with the target nucleic acid would have 77.8% overall complementarity with the target nucleic acid and would thus fall within the scope of the present invention.

Percent complementarity of an antisense oligomeric compound with a region of a target nucleic acid can be determined routinely using BLAST programs (basic local alignment search tools) and PowerBLAST programs known in the art (Altschul et al., J. Mol. Biol., 1990, 215, 403-410; Zhang and Madden, Genome Res., 1997, 7, 649-656). Percent homology, sequence identity or complementarity, can be determined by, for example, the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489). In some embodiments, homology, sequence identity or complementarity, between the antisense oligomeric compound and target is between about 50% to about 60%. In some embodiments, homology, sequence identity or complementarity, is between about 60% to about 70%. In some embodiments, homology, sequence identity or complementarity, is between about 70% and about 80%. In some embodiments, homology, sequence identity or complementarity, is between about 80% and about 90%. In some embodiments, homology, sequence identity or complementarity, is about 90%, about 92%, about 94%, about 95%, about 96%, about 97%, about 98%, about 99% or about 100%.

In some embodiments, “suitable target segments” may be employed in a screen for additional oligomeric compounds that modulate the expression of a selected protein. “Modulators” are those oligomeric compounds that decrease or increase the expression of a nucleic acid molecule encoding a protein and which comprise at least an 8-nucleobase portion which is complementary to a suitable target segment. The screening method comprises the steps of contacting a suitable target segment of a nucleic acid molecule encoding a protein with one or more candidate modulators, and selecting for one or more candidate modulators which decrease or increase the expression of a nucleic acid molecule encoding a protein. Once it is shown that the candidate modulator or modulators are capable of modulating (e.g. either decreasing or increasing) the expression of a nucleic acid molecule encoding a peptide, the modulator may then be employed in further investigative studies of the function of the peptide, or for use as a research, diagnostic, or therapeutic agent in accordance with the present invention.

The suitable target segments of the present invention may also be combined with their respective complementary antisense oligomeric compounds of the present invention to form stabilized double stranded (duplexed) oligonucleotides. Such double stranded oligonucleotide moieties have been shown in the art to modulate target expression and regulate translation as well as RNA processsing via an antisense mechanism. Moreover, the double stranded moieties may be subject to chemical modifications (Fire et al., Nature, 1998, 391, 806-811; Timmons and Fire, Nature 1998, 395, 854; Timmons et al., Gene, 2001, 263, 103-112; Tabara et al., Science, 1998, 282, 430-431; Montgomery et al., Proc. Natl. Acad. Sci. USA, 1998, 95, 15502-15507; Tuschl et al., Genes Dev., 1999, 13, 3191-3197; Elbashir et al., Nature, 2001, 411, 494-498; Elbashir et al., Genes Dev. 2001, 15, 188-200). For example, such double stranded moieties have been shown to inhibit the target by the classical hybridization of antisense strand of the duplex to the target, thereby triggering enzymatic degradation of the target (Tijsterman et al., Science, 2002, 295, 694-697).

The oligomeric compounds of the present invention can also be applied in the areas of drug discovery and target validation. The present invention comprehends the use of the oligomeric compounds and targets identified herein in drug discovery efforts to elucidate relationships that exist between proteins and a disease state, phenotype, or condition. These methods include detecting or modulating a target peptide comprising contacting a sample, tissue, cell, or organism with the oligomeric compounds of the present invention, measuring the nucleic acid or protein level of the target and/or a related phenotypic or chemical endpoint at some time after treatment, and optionally comparing the measured value to a non-treated sample or sample treated with a further oligomeric compound of the invention. These methods can also be performed in parallel or in combination with other experiments to determine the function of unknown genes for the process of target validation or to determine the validity of a particular gene product as a target for treatment or prevention of a particular disease, condition, or phenotype.

The oligomeric compounds of the present invention can be utilized for diagnostics, therapeutics, prophylaxis and as research reagents and kits. Furthermore, antisense oligonucleotides, which are able to inhibit gene expression with exquisite specificity, are often used by those of ordinary skill to elucidate the function of particular genes or to distinguish between functions of various members of a biological pathway.

For use in kits and diagnostics, the oligomeric compounds of the present invention, either alone or in combination with other oligomeric compounds or therapeutics, can be used as tools in differential and/or combinatorial analyses to elucidate expression patterns of a portion or the entire complement of genes expressed within cells and tissues.

As one nonlimiting example, expression patterns within cells or tissues treated with one or more antisense oligomeric compounds are compared to control cells or tissues not treated with antisense oligomeric compounds and the patterns produced are analyzed for differential levels of gene expression as they pertain, for example, to disease association, signaling pathway, cellular localization, expression level, size, structure or function of the genes examined. These analyses can be performed on stimulated or unstimulated cells and in the presence or absence of other compounds and or oligomeric compounds which affect expression patterns.

Examples of methods of gene expression analysis known in the art include DNA arrays or microarrays (Brazma and Vilo, FEBS Lett., 2000, 480, 17-24; Celis, et al., FEBS Lett., 2000, 480, 2-16), SAGE (serial analysis of gene expression)(Madden, et al., Drug Discov. Today, 2000, 5, 415-425), READS (restriction enzyme amplification of digested cDNAs) (Prashar and Weissman, Methods Enzymol., 1999, 303, 258-72), TOGA (total gene expression analysis) (Sutcliffe, et al., Proc. Natl. Acad. Sci. U.S.A., 2000, 97, 1976-81), protein arrays and proteomics (Celis, et al., FEBS Lett., 2000, 480, 2-16; Jungblut, et al., Electrophoresis, 1999, 20, 2100-10), expressed sequence tag (EST) sequencing (Celis, et al., FEBS Lett., 2000, 480, 2-16; Larsson, et al., J. Biotechnol., 2000, 80, 143-57), subtractive RNA fingerprinting (SuRF) (Fuchs, et al., Anal. Biochem., 2000, 286, 91-98; Larson, et al., Cytometry, 2000, 41, 203-208), subtractive cloning, differential display (DD) (Jurecic and Belmont, Curr. Opin. Microbiol., 2000, 3, 316-21), comparative genomic hybridization (Carulli, et al., J. Cell Biochem. Suppl., 1998, 31, 286-96), FISH (fluorescent in situ hybridization) techniques (Going and Gusterson, Eur. J. Cancer, 1999, 35, 1895-904) and mass spectrometry methods (To, Comb. Chem. High Throughput Screen, 2000, 3, 235-41).

The oligomeric compounds of the invention are useful for research and diagnostics, in one aspect because they hybridize to nucleic acids encoding proteins. For example, oligonucleotides that are shown to hybridize with such efficiency and under such conditions as disclosed herein as to be effective protein inhibitors will also be effective primers or probes under conditions favoring gene amplification or detection, respectively. These primers and probes are useful in methods requiring the specific detection of nucleic acid molecules encoding proteins and in the amplification of the nucleic acid molecules for detection or for use in further studies. Hybridization of the antisense oligonucleotides, particularly the primers and probes, of the invention with a nucleic acid can be detected by means known in the art. Such means may include conjugation of an enzyme to the oligonucleotide, radiolabelling of the oligonucleotide or any other suitable detection means. Kits using such detection means for detecting the level of selected proteins in a sample may also be prepared.

The specificity and sensitivity of antisense is also harnessed by those of skill in the art for therapeutic uses. Antisense oligomeric compounds have been employed as therapeutic moieties in the treatment of disease states in animals, including humans. Antisense oligonucleotide drugs, including ribozymes, have been safely and effectively administered to humans and numerous clinical trials are presently underway. It is thus established that antisense oligomeric compounds can be useful therapeutic modalities that can be configured to be useful in treatment regimes for the treatment of cells, tissues and animals, especially humans.

For therapeutics, an animal, such as a human, suspected of having a disease or disorder which can be treated by modulating the expression of a selected protein is treated by administering antisense oligomeric compounds in accordance with this invention. For example, in one non-limiting embodiment, the methods comprise the step of administering to the animal in need of treatment, a therapeutically effective amount of a protein inhibitor. The protein inhibitors of the present invention effectively inhibit the activity of the protein or inhibit the expression of the protein. In some embodiments, the activity or expression of a protein in an animal or cell is inhibited by at least about 10%, at least about 20%, at least about 30%, at least about 40%, at least about 50%, at least about 60%, at least about 70%, at least about 80%, at least about 90%, at least about 95%, at least about 99%, or by 100%.

For example, the reduction of the expression of a protein may be measured in serum, adipose tissue, liver or any other body fluid, tissue or organ of the animal. The cells contained within the fluids, tissues or organs being analyzed can contain a nucleic acid molecule encoding a protein and/or the protein itself.

The oligomeric compounds of the invention can be utilized in pharmaceutical compositions by adding an effective amount of an oligomeric compound to a suitable pharmaceutically acceptable diluent or carrier. Use of the oligomeric compounds and methods of the invention may also be useful prophylactically.

In another embodiment, the present invention provides for the use of an oligomeric compound(s) of the invention in the manufacture of a medicament for the treatment of any and all diseases and conditions disclosed herein.

### EXAMPLES GENERAL

The sequences listed in the examples have been annotated to indicate where there are modified nucleosides or internucleoside linkages. All non-annotated nucleosides are β-D-ribonucleosides linked by phosphodiester internucleoside linkages. Phosphorothioate internucleoside linkages are indicated by underlining or subscript “s”. Modified nucleosides are indicated by a subscripted letter following the capital letter indicating the nucleoside. In particular, subscript “f” indicates 2′-fluoro; subscript “m” indicates 2′-O-methyl; subscript “1” indicates LNA; subscript “e” indicates 2′-O-methoxyethyl (MOE); and subscript “t” indicates 4′-thio. For example Um is a modified uridine having a 2′-OCH₃ group. A “d” preceding a nucleoside indicates a deoxynucleoside such as dT which is deoxythymidine. Some of the strands have a 5′-phosphate group designated as “P-”. Bolded and italicized “C” indicates a 5-methyl C ribonucleoside. Where noted next to the ISIS number of a compound, “as” designates the antisense strand, and “s” designates the sense strand of the duplex, with respect to the target sequence.

### Example 1

### Synthesis of Nucleoside Phosphoramidites

The following compounds, including amidites and their intermediates were prepared as described in U.S. Pat. No. 6,426,220 and published PCT WO 02/36743; 5′-O-DMT-thymidine intermediate for 5-methyl dC amidite, 5′-O-DMT-2′-deoxy-5-methyl-cytidine intermediate for 5-methyl-dC amidite, 5′-O-DMT-2′-deoxy-N4-benzoyl-5-methyl-cytidine penultimate intermediate for 5-methyl dC amidite, [5′-O-DMT-2′-deoxy-N⁴-benzoyl-5-methylcytidin-3′-O-yl]-2-cyanoethyl-N,N-diisopropylphosphoramidite (5-methyl dC amidite), 2′-fluorodeoxyadenosine, 2′-fluorodeoxyguanosine, 2′-fluorouridine, 2′-fluorodeoxycytidine, 2′-O-(2-methoxyethyl) modified amidites, 2′-O-(2-methoxyethyl)-5-methyluridine intermediate, 5′-O-DMT-2′-O-(2-methoxyethyl)-5-methyluridine penultimate intermediate, [5′-O-DMT-2′-O-(2-methoxyethyl)-5-methyluridin-3′-O-yl]-2-cyanoethyl-N,N-diisopropylphosphoramidite (MOE T amidite), 5′-O-DMT-2′-O-(2-methoxyethyl)-5-methylcytidine intermediate, 5′-O-DMT-2′-O-(2-methoxyethyl)-N⁴-benzoyl-5-methyl-cytidine penultimate intermediate, [5′-O-DMT-2′-O-(2-methoxyethyl)-N⁴-benzoyl-5-methylcytidin-3′-O-yl]-2-cyanoethyl-N,N-diisopropylphosphoramidite (MOE 5-Me-C amidite), [5′-O-DMT-2′-O-(2-methoxyethyl)-N⁶-benzoyladenosin-3′-O-yl]-2-cyanoethyl-N,N-diisopropylphosphoramidite (MOE A amdite), [5′-O-DMT-2′-O-(2-methoxyethyl)-N⁴-isobutyrylguanosin-3′-O-yl]-2-cyanoethyl-N,N-diisopropylphosphoramidite (MOE G amidite), 2′-O-(aminooxyethyl) nucleoside amidites and 2′-O-(dimethyl-aminooxyethyl) nucleoside amidites, 2′-(dimethylaminooxyethoxy) nucleoside amidites, 5′-O-tert-butyldiphenylsilyl-O²-2′-anhydro-5-methyluridine, 5′-O-tert-Butyldiphenylsilyl-2′-O-(2-hydroxyethyl)-5-methyluridine, 2′-O-([2-phthalimidoxy)ethyl]-5′-t-butyldiphenylsilyl-5-methyluridine, 5′-O-tert-butyldiphenylsilyl-2′-O-[(2-formadoximinooxy)ethyl]-5-methyluridine, 5′-O-tert-butyldiphenylsilyl-2′-O—[N,N dimethylaminooxyethyl]-5-methyl-uridine, 2′-O-(dimethylaminooxyethyl)-5-methyluridine, 5′-O-DMT-2′-O-(dimethylaminooxyethyl)-5-methyluridine, 5′-O-DMT-2′-O-(2-N,N-dimethylaminooxyethyl)-5-methyluridine-3′-[(2-cyanoethyl)-N,N-diisopropylphosphoramidite], 2′-(aminooxyethoxy) nucleoside amidites, N2-isobutyryl-6-O-diphenylcarbamoyl-2′-O-(2-ethylacetyl)-5′-O-DMT-guanosine-3′-[(2-cyanoethyl)-N,N-diisopropylphosphoramidite], 2′-dimethylamino-ethoxyethoxy (2′-DMAEOE) nucleoside amidites, 2′-O-[2(2-N,N-dimethylaminoethoxy)-ethyl]-5-methyl uridine, 5′-O-DMT-2′-O-[2(2-N,N-dimethylaminoethoxy)-ethyl)]-5-methyl uridine and 5′-O-DMT-2′-O-[2(2-N,N-dimethylaminoethoxy)-ethyl)]-5-methyl uridine-3′-O-(cyanoethyl-N,N-diisopropyl)phosphoramidite.

### Example 2

### Oligonucleotide and Oligonucleoside Synthesis

The oligomeric compounds used in accordance with this invention may be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed. It is well known to use similar techniques to prepare oligonucleotides such as the phosphorothioates and alkylated derivatives.

Oligonucleotides: Unsubstituted and substituted phosphodiester (P═O) oligonucleotides are synthesized on an automated DNA synthesizer (Applied Biosystems model 394) using standard phosphoramidite chemistry with oxidation by iodine.

Phosphorothioates (P═S) are synthesized similar to phosphodiester oligonucleotides with the following exceptions: thiation was effected by utilizing a 10% w/v solution of 3,H-1,2-benzodithiole-3-one 1,1-dioxide in acetonitrile for the oxidation of the phosphite linkages. The thiation reaction step time was increased to 180 sec and preceded by the normal capping step. After cleavage from the CPG column and deblocking in concentrated ammonium hydroxide at 55° C. (12-16 hr), the oligonucleotides were recovered by precipitating with >3 volumes of ethanol from a 1 M NH₄OAc solution. Phosphinate oligonucleotides are prepared as described in U.S. Pat. No. 5,508,270, herein incorporated by reference.

Alkyl phosphonate oligonucleotides are prepared as described in U.S. Pat. No. 4,469,863, herein incorporated by reference.

3′-Deoxy-3′-methylene phosphonate oligonucleotides are prepared as described in U.S. Pat. No. 5,610,289 or 5,625,050, herein incorporated by reference.

Phosphoramidite oligonucleotides are prepared as described in U.S. Pat. No., 5,256,775 or U.S. Pat. No. 5,366,878, herein incorporated by reference.

Alkylphosphonothioate oligonucleotides are prepared as described in published PCT applications PCT/US94/00902 and PCT/US93/06976 (published as WO 94/17093 and WO 94/02499, respectively), herein incorporated by reference.

3′-Deoxy-3′-amino phosphoramidate oligonucleotides are prepared as described in U.S. Pat. No. 5,476,925, herein incorporated by reference.

Phosphotriester oligonucleotides are prepared as described in U.S. Pat. No. 5,023,243, herein incorporated by reference.

Borano phosphate oligonucleotides are prepared as described in U.S. Pat. Nos. 5,130,302 and 5,177,198, both herein incorporated by reference.

Oligonucleosides: Methylenemethylimino linked oligonucleosides, also identified as MMI linked oligonucleosides, methylenedimethylhydrazo linked oligonucleosides, also identified as MDH linked oligonucleosides, and methylenecarbonylamino linked oligonucleosides, also identified as amide-3 linked oligonucleosides, and methyleneaminocarbonyl linked oligonucleosides, also identified as amide-4 linked oligonucleosides, as well as mixed backbone oligomeric compounds having, for instance, alternating MMI and P═O or P═S linkages are prepared as described in U.S. Pat. Nos. 5,378,825, 5,386,023, 5,489,677, 5,602,240 and 5,610,289, all of which are herein incorporated by reference.

Formacetal and thioformacetal linked oligonucleosides are prepared as described in U.S. Pat. Nos. 5,264,562 and 5,264,564, herein incorporated by reference.

Ethylene oxide linked oligonucleosides are prepared as described in U.S. Pat. No. 5,223,618, herein incorporated by reference.

### Example 3

### RNA Synthesis

In general, RNA synthesis chemistry is based on the selective incorporation of various protecting groups at strategic intermediary reactions. Although one of ordinary skill in the art will understand the use of protecting groups in organic synthesis, a useful class of protecting groups includes silyl ethers. In particular bulky silyl ethers are used to protect the 5′-hydroxyl in combination with an acid-labile orthoester protecting group on the 2′-hydroxyl. This set of protecting groups is then used with standard solid-phase synthesis technology. It is important to lastly remove the acid labile orthoester protecting group after all other synthetic steps. Moreover, the early use of the silyl protecting groups during synthesis ensures facile removal when desired, without undesired deprotection of 2′ hydroxyl.

Following this procedure for the sequential protection of the 5′-hydroxyl in combination with protection of the 2′-hydroxyl by protecting groups that are differentially removed and are differentially chemically labile, RNA oligonucleotides were synthesized.

RNA oligonucleotides are synthesized in a stepwise fashion. Each nucleotide is added sequentially (3′- to 5′-direction) to a solid support-bound oligonucleotide. The first nucleoside at the 3′-end of the chain is covalently attached to a solid support. The nucleotide precursor, a ribonucleoside phosphoramidite, and activator are added, coupling the second base onto the 5′-end of the first nucleoside. The support is washed and any unreacted 5′-hydroxyl groups are capped with acetic anhydride to yield 5′-acetyl moieties. The linkage is then oxidized to the more stable and ultimately desired P(V) linkage. At the end of the nucleotide addition cycle, the 5′-silyl group is cleaved with fluoride. The cycle is repeated for each subsequent nucleotide.

Following synthesis, the methyl protecting groups on the phosphates are cleaved in 30 minutes utilizing 1 M disodium-2-carbamoyl-2-cyanoethylene-1,1-dithiolate trihydrate (S₂Na₂) in DMF. The deprotection solution is washed from the solid support-bound oligonucleotide using water. The support is then treated with 40% methylamine in water for 10 minutes at 55° C. This releases the RNA oligonucleotides into solution, deprotects the exocyclic amines, and modifies the 2′-groups. The oligonucleotides can be analyzed by anion exchange HPLC at this stage.

The 2′-orthoester groups are the last protecting groups to be removed. The ethylene glycol monoacetate orthoester protecting group developed by Dharmacon Research, Inc. (Lafayette, Colo.), is one example of a useful orthoester protecting group which, has the following important properties. It is stable to the conditions of nucleoside phosphoramidite synthesis and oligonucleotide synthesis. However, after oligonucleotide synthesis the oligonucleotide is treated with methylamine which not only cleaves the oligonucleotide from the solid support but also removes the acetyl groups from the orthoesters. The resulting 2-ethyl-hydroxyl substituents on the orthoester are less electron withdrawing than the acetylated precursor. As a result, the modified orthoester becomes more labile to acid-catalyzed hydrolysis. Specifically, the rate of cleavage is approximately 10 times faster after the acetyl groups are removed. Therefore, this orthoester possesses sufficient stability in order to be compatible with oligonucleotide synthesis and yet, when subsequently modified, permits deprotection to be carried out under relatively mild aqueous conditions compatible with the final RNA oligonucleotide product.

Additionally, methods of RNA synthesis are well known in the art (Scaringe, S. A. Ph.D. Thesis, University of Colorado, 1996; Scaringe, S. A., et al., J. Am. Chem. Soc., 1998, 120, 11820-11821; Mafteucci, M. D. and Caruthers, M. H. J. Am. Chem. Soc., 1981, 103, 3185-3191; Beaucage, S. L. and Caruthers, M. H. Tetrahedron Lett., 1981, 22, 1859-1862; Dahl, B. J., et al., Acta Chem. Scand. 1990, 44, 639-641; Reddy, M. P., et al., Tetrahedrom Lett., 1994, 25, 4311-4314; Wincott, F. et al., Nucleic Acids Res., 1995, 23, 2677-2684; Griffin, B. E., et al., Tetrahedron, 1967, 23, 2301-2313; Griffin, B. E., et al., Tetrahedron, 1967, 23, 2315-2331).

RNA antisense oligomeric compounds (RNA oligonucleotides) of the present invention can be synthesized by the methods herein or purchased from Dharmacon Research, Inc (Lafayette, Colo.). Once synthesized, complementary RNA antisense oligomeric compounds can then be annealed by methods known in the art to form double stranded (duplexed) antisense oligomeric compounds. For example, duplexes can be formed by combining 30 μl of each of the complementary strands of RNA oligonucleotides (50 uM RNA oligonucleotide solution) and 15 μL of 5× annealing buffer (100 mM potassium acetate, 30 mM HEPES-KOH pH 7.4, 2 mM magnesium acetate) followed by heating for 1 minute at 90° C., then 1 hour at 37° C. The resulting duplexed antisense oligomeric compounds can be used in kits, assays, screens, or other methods to investigate the role of a target nucleic acid.

### Example 4

### Synthesis of Chimeric Oligonucleotides

Chimeric oligonucleotides having 2′-O-alkyl phosphorothioate and 2′-deoxy phosphorothioate oligonucleotide segments are synthesized using an Applied Biosystems automated DNA synthesizer Model 394, as above. Oligonucleotides are synthesized using the automated synthesizer and 2′-deoxy-5′-dimethoxytrityl-3′-O-phosphoramidite for the DNA portion and 5′-dimethoxytrityl-2′-O-methyl-3′-O-phosphoramidite for 5′ and 3′ wings. The standard synthesis cycle is modified by incorporating coupling steps with increased reaction times for the 5′-dimethoxytrityl-2′-O-methyl-3′-O-phosphoramidite. The fully protected oligonucleotide is cleaved from the support and deprotected in concentrated ammonia (NH₄OH) for 12-16 hr at 55° C. The deprotected oligo is then recovered by an appropriate method (precipitation, column chromatography, volume reduced in vacuo and analyzed spetrophotometrically for yield and for purity by capillary electrophoresis and by mass spectrometry.

[2′-O-(2-methoxyethyl)]-[2′-deoxy]-[-2′-O-(methoxyethyl)] chimeric phosphorothioate oligonucleotides were prepared as per the procedure above for the 2′-O-methyl chimeric oligonucleotide, with the substitution of 2′-O-(methoxyethyl) amidites for the 2′-O-methyl amidites. [2′-O-(2-Methoxyethyl) Phosphodiester]-[2′-deoxy Phosphorothioate]-[2′-O-(2-Methoxyethyl) Phosphodiester] Chimeric Oligonucleotides

[2′-O-(2-methoxyethyl phosphodiester]-[2′-deoxy phosphorothioate]-[2′-O-(methoxyethyl) phosphodiester] chimeric oligonucleotides are prepared as per the above procedure for the 2′-O-methyl chimeric oligonucleotide with the substitution of 2′-O-(methoxyethyl) amidites for the 2′-O-methyl amidites, oxidation with iodine to generate the phosphodiester internucleotide linkages within the wing portions of the chimeric structures and sulfurization utilizing 3,H-1,2 benzodithiole-3-one 1,1 dioxide (Beaucage Reagent) to generate the phosphorothioate internucleotide linkages for the center gap. Other chimeric oligonucleotides, chimeric oligonucleosides and mixed chimeric oligonucleotides/oligonucleosides are synthesized according to U.S. Pat. No. 5,623,065, herein incorporated by reference.

### Example 5

### Preparation of Compound II

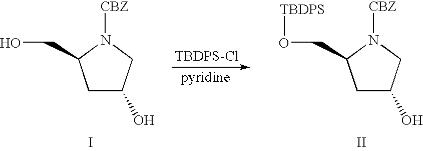

N-CBZ-trans-4-hydroxy-L-prolinol (Compound I, 1.5 g, 6 mmol) was dissolved in pryidine and cooled to 0° C. tert-Butyldiphenylsilyl chloride (1.8 g, 6.6 mmol) was added over 5 minutes and the mixture stirred at room temperature for 18 hours. The solvent was then removed in vacuo and the product purified by column chromatography (silica gel, hexane:ethyl acetate (1:1) eluent) to give Compound II (1.7 g, 58%).

### Example 6

### Preparation of Compound III

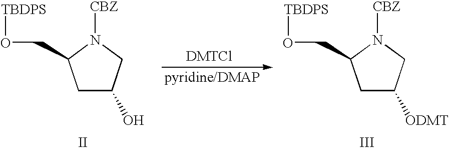

Compound II (1.7 g, 3.48 mmol) was dissolved in pyridine. Dimethoxytrityl chloride (DMTCl) (1.178 g, 3.48 mmol) and dimethylaminopyridine (DMAP, 10 mL) were added and the mixture stirred at room temperature overnight. The solvent was removed in vacuo and the product purified by column chromatography (silica gel, hexane:ethyl acetate (1:4) eluent) to give Compound III.

### Example 7

### Preparation of Compound IV

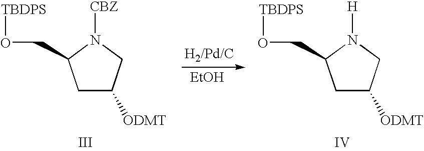

Palladium on carbon (70 mg, 10%) was added to a solution of Compound III (all the purified product from previous step) in ethanol (100 mL), under an atmosphere of hydrogen and the mixture stirred at room temperature overnight. The Pd/C was removed by filtration, the ethanol removed in vacuo and the product purified by column chromatography (silica gel, methanol:ethyl acetate (5:95) eluent) to give Compound IV (11.0 g, 81%).

### Example 8

### Preparation of Compound V

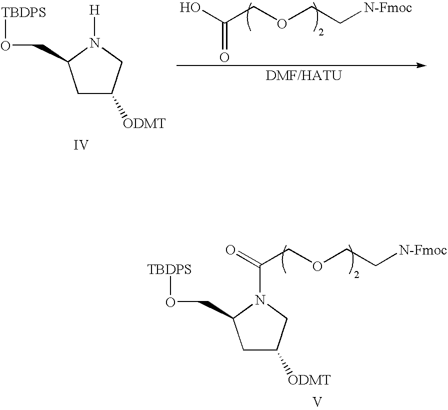

2-(7-Aza-1H-benzotriazole-1-yl)-1,1,3,3-tetramethyluronium hexafluorophosphate (HATU) (695 mg, 1.83 mmol) was added to a solution of 2-Fmoc-aminoethylether-(2-carboxymethylether (645 mg, 1.67 mmol) in dimethylformaminde (DMF, 3 mL), and allowed to stir at room temperature for 5 minutes. The mixture was added to a solution of Compound IV (˜1 g) in DMF (1 mL), and stirred at room temperature for 16 hours. The solvent was removed in vacuo, the residue dissolved in ethylacetate and filtered through a silica plug using hexane/ethylacetate (1/1) as the eluent. The ethyl acetate was removed in vacuo to give compound V.

### Example 9

### Preparation of Compound VI

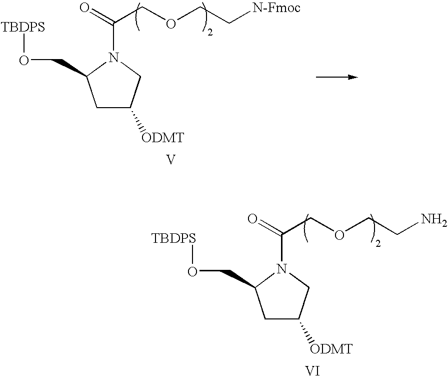

The crude product V from the previous step was dissolved in 20% piperidine/DMF (5 mL) and stirred at room temperature for 2 hours. The solvent was then removed in vacuo and the product purified by column chromatography (silica gel, 4% triethylamine in methanol/ethylacetate (15/85) eluent) to give the desired product VI (1.1 g, 82%).

### Example 10

### Preparation of Compound VII

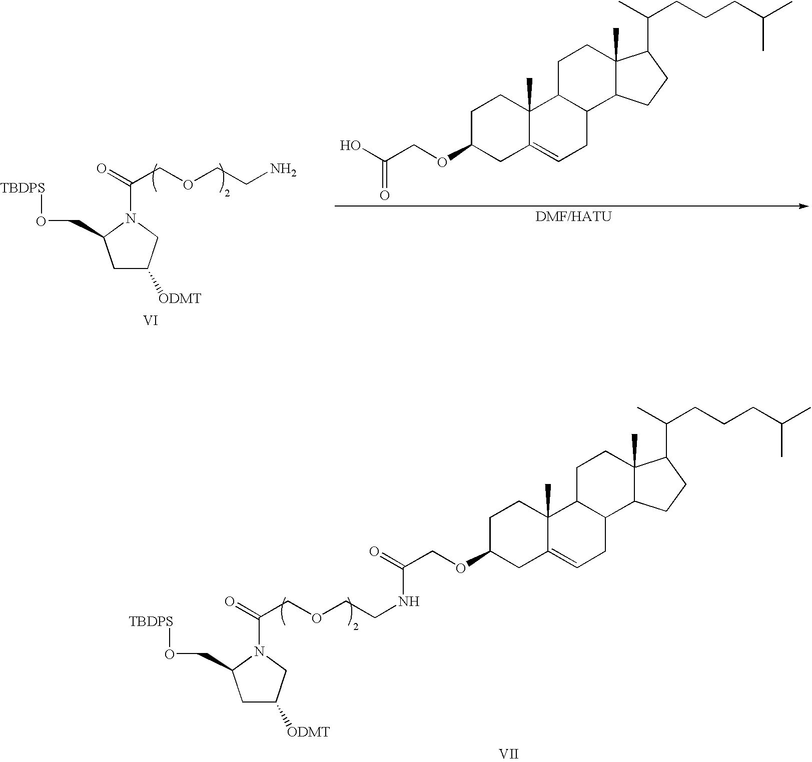

2-(7-Aza-1H-benzotriazole-1-yl)-1,1,3,3-tetramethyluronium hexafluorophosphate (HATU) (695 mg, 1.83 mg) was added to a solution of carboxymethyl cholesterol (1.37 mmol, 1.1 eq, 609 mg) (645 mg, 1.67 mmol) in dimethylformaminde (DMF, 3 mL), and allowed to stir at room temperature for 5 minutes. The mixture was added to a solution of Compound VI (˜1 g) in DMF (1 mL), and stirred at room temperature for 16 hours. The solvent was removed in vacuo, the residue dissolved in ethylacetate and filtered through a silica plug using hexane/ethylacetate (1/1) as the eluent. The ethyl acetate was removed in vacuo to give compound VII (0.98 mmol, 78%).

### Example 11

### Preparation of Compound VIII

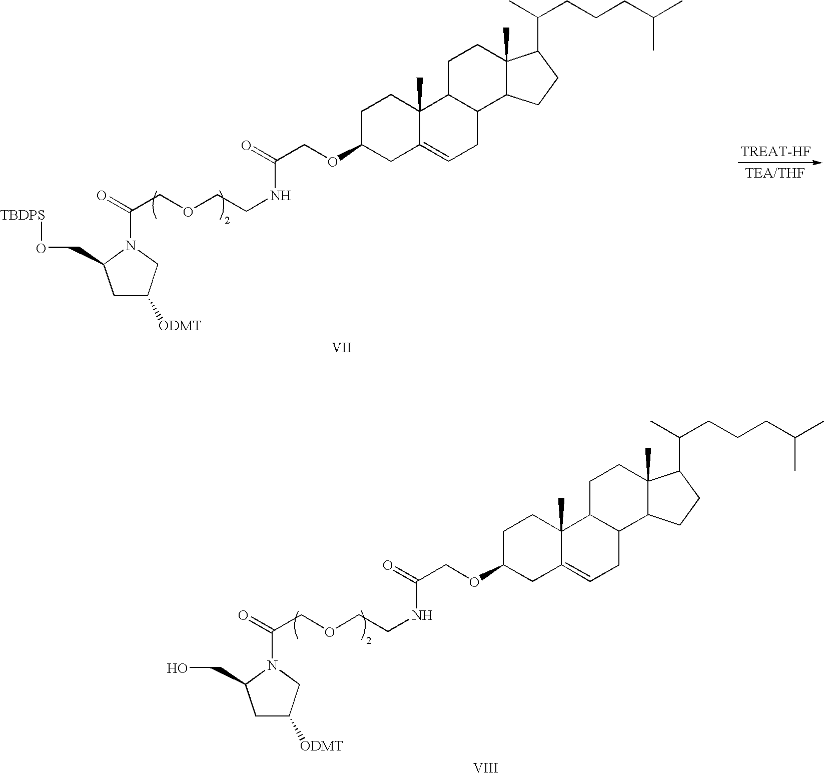

To a solution of TREAT-HF (815 μL, 5 mmol), triethylamine (350 μL, 2.5 mmol) and DMF (10 mL) was added to Compound VII (1.2 g, 1 mmol), and the mixture stirred at room temperature for 18 hours. After filtration through silica, using 5% methanol in ethylactetate as eluent, the solvent was removed in vacuo and the resulting oil purified by column chromatography on silica gel (methanol:ethyl acetate (5:95) eluent) to give Compound VIII as a white solid (0.71 g, 72%).

### Example 12

### Preparation of 1-N-[2-[2-[(cholesteroyl)acetyl]aminoethoxy]ethoxy]acetyl]-4-O-(dimethoxytrityl)-2-O-(succinyl-CPG-methyl)pyrrolidine (Compound IX)

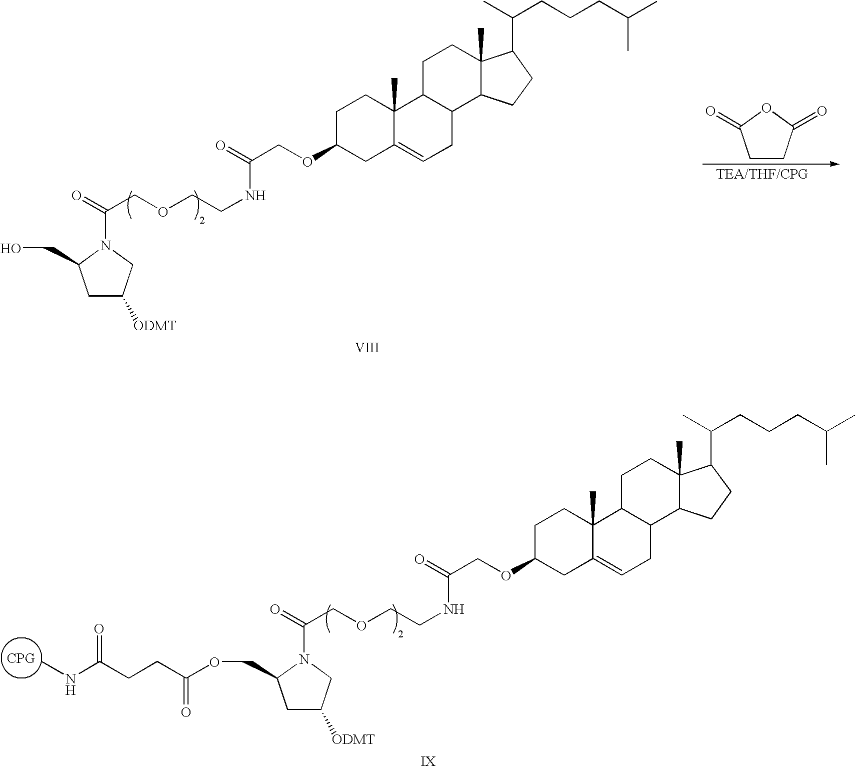

Compound VIII (0.4 g, 0.4 mmol) was mixed with succinic anhydride (0.8 g, 0.8 mmol) and DMAP (0.02 g, 0.2 mmol) and dried over P₂O₅ under reduced pressure over night. The mixture was dissolved in anhydrous 1,2-dichloroethane (1.2 mL) and triethyl amine (0.22 mL, 1.6 mmol) was added. The reaction mixture was heated at 60° C. for 4 h. The reaction mixture was diluted with dichloromethane (25 mL) and washed with 5% aqueous citric acid (25 mL) and brine (25 mL). The organic phase separated and dried over anhydrous Na₂SO₄ and concentrated under reduced pressure. The residue obtained was purified by silica gel column chromatography and eluted with 5% methanol in dichloromethane. The succinyl derivative (0.36 g) was loaded on to the aminoalkyl controlled pore glass (CPG) according to the standard synthetic procedure (TBTU mediated synthesis of functionalized CPG synthesis: Bayer, E.; Bleicher, K.; Maier, M. A.; Z. Naturforsch. 1995, 50b, 1096-1100) to yield the functionalized solid support (64.72 μmol/g).

### Example 13

### Preparation of 1-N-[2-[2-[(cholesteroyl)acetyl]aminoethoxy]ethoxy]acetyl]-4-O-(dimethoxytrityl)-2-methyl-[(2-cyanoethyl)-N,N-diisopropyl]phosphoramidite]-pyrrolidine (X)

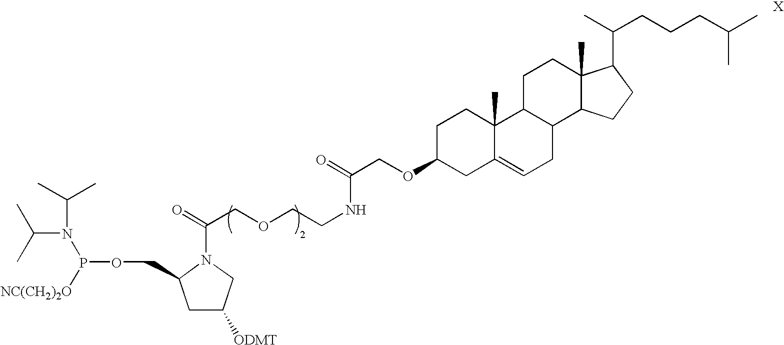

Compound VIII (1.0 g, 1.0 mmol) was mixed with N,N-diisopropylamine tetrazolide (0.7 g, 1.0 mmol) and dried over P₂O₅ in vacuum overnight at 40° C. The mixture was dissolved in anhydrous CH₃CN (5 mL) and 2-cyanoethyl N,N,N′N′-tetraisopropylphosphorodiamidite (0.63 mL, 2 mmol) was added dropwise. The reaction mixture was stirred at room temperature under an argon atmosphere for 6 hours. The solvent was removed under reduced pressure. The residue was purified by silica gel flash flash column chromatography (ethyl acetate) to afford Compound X (0.65 g, 54.7%) as an oil. ³¹P NMR (80 MHz, CDCl₃) δ 149.24, 149.17; MS (FAB) m/z 1175.9 (M−H)⁻.

### Example 14

Cholesterol Conjugated RNA

Oligoribonucleotide SEQ ID NO: 1 (ISIS No. 366559) having a cholesterol group conjugated using a pyrrolidinyl linker of the invention was synthesized using the cholesterol functionalized solid support prepared in a previous example using a DNA/RNA synthesizer. Solutions containing 0.12 M amidites in anhydrous acetonitrile were used for the synthesis of the modified oligoribonucleotides. The phosphoramidite solutions were delivered in two portions, each followed by a 5 min coupling wait time. The standard 2′-O-TBDMS phosphoramidites (Glen Research Inc.) were used for the incorporation of A, C, G and U residues. Oxidation of the internucleotide phosphite triester to phosphate triester was carried out using tert-butylhydroperoxide/acetonitrile/water (10:87:3) with a wait time of 10 min. All other steps in the protocol supplied by the manufacturer were used without modifications. The coupling efficiencies were more than 97%. After completion of the synthesis, solid support was suspended in aqueous ammonium hydroxide (30 wt. %): ethanol (2:1) and heated at 55° C. for 6 h to complete the removal of all protecting groups except the TBDMS group at 2′-position. The solid support was filtered and the filtrate was evaporated under reduced pressure. The residue obtained was re-suspended in anhydrous TEA.HF/TEA/NMP solution (1 mL of a solution of 1.5 mL N-methylpyrrolidine, 750 μl TEA and 1 ml of TEA 3HF to provide a 1.4 M HF concentration) and heated at 65° C. for 1.5 h to remove the 2′-TBDMS groups. The reaction was quenched with 1.5 M ammonium bicarbonate (1 mL) and the mixture was loaded on to a Sephadex G-25 column (NAP Columns, Amersham Biosciences Inc.). The oligonucleotides were eluted with water and the fractions containing the oligonucleotides were pooled together and purified by HPLC (Waters, C-4, 7.8×300 mm, delta pack, 15 μm, 300 A°, A=100 mM ammonium acetate, pH=7, B=acetonitrile, 5 to 20% B in 70 min, then 80% Bin 85 min, Flow 2.5 mL min⁻¹, λ=260 nm). Fractions containing full-length oligonucleotides were pooled together (assessed by CGE analysis >90%) and evaporated. The residue was dissolved in sterile water (0.3 mL) solution. Ethanol (1 mL) was added and cooled to −78° C. for 1 h to get a precipitate which was pelleted out using a microfuge (NYCentrifuge 5415C; Eppendorf, Westbury, N.Y.) at 3000 rpm (735 g) for 15 min. The pellets were collected by decanting the supernatant. The oligonucleotides were characterized by ES MS analysis and purity was assessed by capillary gel electrophoresis.

### Example 15

### Cholesterol Conjugated Alternating 2′-F 2′-O-Methyl Modified Oligonucleotides

The oligonucleotides SEQ ID NO: 2 (ISIS No. 366561) and SEQ ID NO: 1 (ISIS No. 366667) were synthesized using solid support linked cholesterol conjugated pyrrolidinyl group and the phosphoramidite functionalized cholesterol conjugated pyrrolidinyl group respectively, both prepared in a previous example, on DNA/RNA synthesizer. The 2′-deoxy-2′-fluoro and 2′-O-methyl modified A, C, G and U phosphoramidites were used for the incorporation of corresponding residues. A universal linker loaded solid support (Guzaev, A. P; Manoharan, M. J. Am. Chem. Soc. 2003, 125, 2380-2381) was used for the synthesis of oligonucleotide ISIS No. 366667. A 0.1 M solution of amidites in anhydrous acetonitrile was used for the synthesis of the modified oligoribonucleotides. The phosphoramidite solutions were delivered in two portions, each followed by a 5 min coupling wait time. Oxidation of the internucleotide phosphite triester to phosphate triester was carried out using tert-butylhydroperoxide/acetonitrile/water (10:87:3) with a wait time of 10 min. All other steps in the protocol supplied by instrument manual were used without modifications. The coupling efficiencies were more than 97%. The final DMT group was group was removed during the synthesis on the synthesizer. The solid supports were suspended in aqueous ammonium hydroxide (30 wt. %): ethanol (2:1) and heated at 55° C. for 6 h to complete the removal of all protecting groups. The solid support was filtered and the filtrate was concentrated. The residue obtained was purified by HPLC (Waters, C-4, 7.8×300 mm, delta pack, 15 μm, 300 A°, A=100 mM ammonium acetate, pH=7, B=acetonitrile, 5 to 20% B in 70 min, then 80% Bin 85 min, Flow 2.5 mL min⁻¹, λ=260 nm). Fractions containing full-length oligonucleotides were pooled together (assessed by CGE analysis >90%) and evaporated. The oligonucleotides were characterized by ES MS analysis and purity was assessed by capillary gel electrophoresis.

| SEQ ID NO/ | |
|---|---|
| ISIS No. | Sequence |
| 1/366559 s | 5′-AAGUAAGGACCAGAGACAA-Chol-3′ |
| 1/366667 s | 5′-Chol-A_{f}AₘG_{f}UₘA_{f}AₘG_{f}GₘA_{f}CₘC_{f}AₘG_{f}Aₘ |
| | G_{f}AₘC_{f}AₘA_{f}-3′ |
| 2/366561 as | 3′-Chol-UₘU_{f}CₘA_{f}UₘU_{f}CₘC_{f}UₘG_{f}GₘU_{f}CₘU_{f} |
| | CₘU_{f}GₘU_{f}Uₘ-P-5′ |
| 1/341401 s | 5′-AAGUAAGGACCAGAGACAA-3′ |
| 2/341391 as | 3′-UUCAUUCCUGGUCUCUGUU-5′ |
| 1/359996 s | 5′-A_{f}AₘG_{f}UₘA_{f}AₘG_{f}GₘA_{f}CₘC_{f}AₘG_{f}AₘG_{f}Aₘ |
| | C_{f}AₘA_{f}-3′ |
| 2/359995 as | 3′-UₘU_{f}CₘA_{f}UₘU_{f}CₘC_{f}UₘG_{f}GₘU_{f}CₘU_{f}CₘU_{f} |
| | GₘU_{f}Uₘ-5′ |
| 1/366667 s | 5′-Chol-A_{f}AₘG_{f}UₘA_{f}AₘG_{f}GₘA_{f}CₘC_{f}AₘG_{f}Aₘ |
| | G_{f}AₘC_{f}AₘA_{f}-3′ |
| 2/35999 as5 | 3′-UₘU_{f}CₘA_{f}UₘU_{f}CₘC_{f}UₘG_{f}GₘU_{f}CₘU_{f}CₘU_{f}Gₘ |
| | U_{f}Uₘ-5′ |
| 1/3599965 | 5′-A_{f}AₘG_{f}UₘA_{f}AₘG_{f}GₘA_{f}CₘC_{f}AₘG_{f}AₘG_{f}AₘC_{f} |
| | AₘA_{f}-3′ |
| 2/366561 as | 3′-Chol-UₘU_{f}CₘA_{f}UₘU_{f}CₘC_{f}UₘG_{f}GₘU_{f}CₘU_{f} |
| | CₘU_{f}GₘU_{f}Uₘ-P-5′ |
| 1/366559 s | 5′-AAGUAAGGACCAGAGACAA-Chol-3′ |
| 2/359550 as | 3′-UUₛCAₛUUₛCCₛUGₛGUₛCUₛCUₛGUₛU-5′ |

(alternating P = S, subscript s) s = sense strand, as = antisense strand

| | Calcd | Found | Purity % |
|---|---|---|---|
| SEQ ID NO | Mass | Mass | full length |
| 1/366559 s | 6927.9 | 6928.8 | 96 |
| 1/366667 s | 7075.0 | 7074.8 | 93 |
| 2/366561 as | 6798.6 | 6799.6 | 95 |

Thermal Stability of Cholesterol Conjugated siRNA Duplexes

| SEQ ID NOs/ISIS Nos. | Tm |
|---|---|
| 1/341401 s/2/341391 as | 72.8 |
| 1/359996 s/2/359995 as | 93.9 |
| 1/366667 s/2/35999 as | 94.1 |
| 1/359996 s/2/366561 as | 96.6 |
| 1/366559 s/2/359550 as | 74.0. |

### Example 16

### PTEN Assay in Primary Mouse Hepatocytes

Primary mouse hepatocytes were prepared from female Balb/c mice 4-6 weeks of age or older. Primary mouse hepatocytes were cultured in William's E media supplemented to contain about 10% FBS, about 1% penicillin/streptomycin/anti-mitotic, about 1% of a 1M HEPES solution, and about 1% of a 200 mM L-glutamine solution (all GEBCO® cell culture reagents available from Invitrogen Life Technologies, Carlsbad Calif.). Cells were seeded into 96-well plates (Falcon-Primaria #3872) coated with 0.1 mg/mL collagen at a density of approximately 10,000 cells/well for use in oligomeric compound transfection experiments.

Selected oligomeric compounds are prepared and for double stranded compositions, the complementary strands of the duplex are annealed. The single strands are aliquoted and diluted to a concentration of 50 μM. Once diluted, 30 μL of each strand is combined with 15 μL of a 5× solution of annealing buffer. The final concentration of the buffer is 100 mM potassium acetate, 30 mM HEPES-KOH pH 7.4, and 2 mM magnesium acetate. The final volume is 75 μL. This solution is incubated for 1 minute at 90° C. and then centrifuged for 15 seconds. The tube is allowed to sit for 1 hour at 37° C. at which time the duplexes are ready for use in a selected assay. The final concentration of the duplex is 20 μM.

Selected duplex compositions were evaluated for their ability to modulate target PTEN levels. Mouse primary hepatocytes plated at a density of about 10,000 cells/well in 96-well plates were washed once with OPTI-MEM-1™ reduced-serum medium (Gibco BRL) and then treated with duplexes with or without a transfection reagent. For free-uptake experiments, cells were treated with naked duplexes in complete media overnight. For lipid-mediated transfection, cells are treated with 130 μL of OPTI-MEM-1™ containing a ratio of 6 μL of LIPOFECTIN™ per 100 nM duplex per mL of OPTI-MEM medium. After about 5 hours of treatment, the transfection medium is replaced with fresh medium.

Cells were harvested approximately 16 hours after treatment, at which time RNA was isolated and target reduction measured by real-time RT-PCR using the following primer probe set designed to human PTEN: forward primer: AATGGCTAAGTGAAG-ATGACAATCAT (SEQ ID NO: 31, reverse primer: TGCACATATCATTACACCAGTTCGT (SEQ ID NO: 32), and the PCR probe was: FAM-TTGCAGCAATTCACTGTAAAGCTGGAAAGG-TAMRA, where FAM is the fluorescent dye and TAMRA is the quencher dye.

| SEQ ID NO./ | |
|---|---|
| ISIS NO. | Sequence |
| 1/341401 s | 5′-AAGUAAGGACCAGAGACAA-3′ |
| 2/341391 as | 3′-UUCAUUCCUGGUCUCUGUU-5′ |
| 1/366559 s | 5′-AAGUAAGGACCAGAGACAA-Chol-3′ |
| 2/341391 as | 3′-UUCAUUCCUGGUCUCUGUU-5′ |
| 1/366559 s | 5′-AAGUAAGGACCAGAGACAA-Chol-3′ |
| 2/359995 as | 3′-UₘU_{f}CₘA_{f}UₘU_{f}CₘC_{f}UₘG_{f}GₘU_{f}CₘU_{f}CₘU_{f}GₘU_{f}Uₘ5′ |
| 1/366667 s | 5′-Chol-A_{f}AₘG_{f}UₘA_{f}AₘG_{f}GₘA_{f}CₘC_{f}AₘG_{f}AₘG_{f}AₘC_{f}AₘA_{f}-′3′ |
| 2/341391 as | 3 -UUCAUUCCUGGUCUCUGUU-5′ |
| 1/359467 s | 5′-AAGUAAGGACCAGAGACAA-3′ |
| 2/366561 as | 3′-Chol-UₘU_{f}CₘA_{f}UₘU_{f}CₘC_{f}UₘG_{f}GₘU_{f}CₘU_{f}CₘU_{f}GₘU_{f}Uₘ-P-5′ |
| 1/341401 s | 5′-AAGUAAGGACCAGAGACAA-3′ |
| 2/366561 as | 3′-Chol-UₘU_{f}CₘA_{f}UₘU_{f}CₘC_{f}UₘG_{f}GₘU_{f}CₘU_{f}CₘU_{f}GₘU_{f}Uₘ-P-5′ |

| SEQ ID NO./ | Activity (% untreated control) |
|---|---|
| ISIS NO. | (LIPOFECTIN™) |
| 1/341401 s/ 2/341391 as | 20 (300 nM) |
| 1/366559 s/ 2/341391 as | 46 (300 nM) |
| 1/366559 s/ 2/359995 as | 38 (300 nM) |
| 1/366667 s/ 2/341391 as | 24 (300 nM) |
| 1/359467 s/ 2/366561 as | 40 (100 nM) |
| 1/341401 s/ 2/366561 as | 21 (300 nM) |
| SEQ ID NO./ | |
| ISIS NO. | Free uptakeActivity (IC₅₀₎ |
| 1/341401 s/ 2/341391 as | n/a |
| 1/366559 s/ 2/341391 as | n/a |
| 1/366559 s/ 2/359995 as | n/a |
| 1/366667 s/ 2/341391 as | 654.4 |
| 1/359467 s/ 2/366561 as | n/a |
| 1/341401 s/ 2/366561 as | n/a |

| SEQ ID NO./ | |
|---|---|
| ISIS NO. | Sequence |
| 1/359996 s | 5′-A_{f}AₘG_{f}UₘA_{f}AₘG_{f}GₘA_{f}CₘC_{f}AₘG_{f}AₘG_{f}AₘC_{f}AₘA_{f}-3′ |
| 2/359995 as | 3′-UₘU_{f}CₘA_{f}UₘU_{f}CₘC_{f}UₘG_{f}GₘU_{f}CₘU_{f}CₘU_{f}GₘU_{f}Uₘ-5′ |
| 1/366667 s | 5′-Chol-A_{f}AₘG_{f}UₘA_{f}AₘG_{f}GₘA_{f}CₘC_{f}AₘG_{f}AₘG_{f}AₘC_{f}AₘA_{f}-3′ |
| 2/359995 as | 3′-UₘU_{f}CₘA_{f}UₘU_{f}CₘC_{f}UₘG_{f}GₘU_{f}CₘU_{f}CₘU_{f}GₘU_{f}Uₘ-5′ |
| 1/366667 s | 5′-Chol-A_{f}AₘG_{f}UₘA_{f}AₘG_{f}GₘA_{f}CₘC_{f}AₘG_{f}AₘG_{f}AₘC_{f}AₘA_{f}-3′ |
| 2/352820 as | 3′-UₘU_{f}CₘA_{f}UₘU_{f}CₘC_{f}UₘG_{f}GₘU_{f}CₘU_{f}CₘU_{f}GₘU_{f}Uₘ-P-5′ |
| 1/359996 s | 5-′A_{f}AₘG_{f}UₘA_{f}AₘG_{f}GₘA_{f}CₘC_{f}AₘG_{f}AₘG_{f}AₘC_{f}AₘA_{f}-3′ |
| 2/366561 as | 3′-Chol-UₘU_{f}CₘA_{f}UₘU_{f}CₘC_{f}UₘG_{f}GₘU_{f}CₘU_{f}CₘU_{f}GₘU_{f}Uₘ-P-5′ |
| 4/344178 s | 5′-AAGUAAGGACCAGAGACAAA-3′ |
| 5/303912 as | 3′-UUCAUUCCUGGUCUCUGUUA-5′ |
| 3/29592 as | 3′-d(TₑTₑCₑAₑdTdTdCdCdTdGdGdTdCdTCₑTₑGₑTₑ-)5′ |

| SEQ ID NO./ | Activity (transfection with LIPOFECTIN™) |
|---|---|
| ISIS NO. | (data from 300 uM data point as % untreated control) |
| 1/359996 s/ 2/359995 as | 13 |
| 1/366667 s/ 2/359995 as | 20 |
| 1/366667 s/ 2/352820 as | 20 |
| 1/359996 s/ 2/366561 as | 14 |
| 4/344178 s/ 5/303912 as | 41 |
| 3/29592 as | 29 |
| SEQ ID NO./ | |
| ISIS NO. | Free uptakeActivity (IC₅₀ ) |
| 1/359996 s/ 2/359995 as | n/a |
| 1/366667 s/ 2/359995 as | 248.3 |
| 1/366667 s/ 2/352820 as | 212.1 |
| 1/359996 s/ 2/366561 as | 717.9 |
| 4/344178 s/ 5/303912 as | 319.6 |
| 3/29592 as | n/a. |

### Example 17

### In Vitro PTEN Assay of siRNAs Having a Pyrrolidinyl Conjugated Cholesterol Group in Hela Cells

In accordance with the present invention, a series of oligomeric compounds were synthesized and tested for their ability to reduce target expression over a range of doses relative to an unmodified compound. Various double strand siRNA's and a single strand 18 mer 4-10-4 MOE gapmer (MOE=2′-methoxyethoxy modified) were prepared for comparison. Cholesterol was conjugated to selected siRNA's using the pyrrolidinyl group. The siRNA's were modified having alternating 2′-OCH₃ and 2′-F modified nucleosides.

HeLa cells were treated with the single and double stranded oligomeric compounds (siRNA constructs) shown below at concentrations of 0, 0.15, 1.5, 15, and 150 nM using methods described herein. The nucleosides are annotated as to chemical modification as per the legend at the beginning of the examples. Expression levels of human PTEN were determined by quantitative real-time PCR and normalized to RIBOGREEN™ as described in other examples herein. Resulting dose-response curves were used to determine the IC₅₀ for each treatment.

| SEQ ID NO./ | | |
|---|---|---|
| ISIS NO. | Sequence | IC50 |
| 3/29592 as | 3′-d(TₑTₑCₑAₑdTdTdCdCdTdGdGdTdCdTCₑTₑGₑTₑ-)5′ | 71.6(ss) |
| 1/359996 s | 5′-A_{f}AₘG_{f}UA_{f}AG_{f}GA_{f}CC_{f}AG_{f}AG_{f}AC_{f}AA_{f}-3′ | 0.035 |
| 2/359995 as | 3′-UₘUCₘA_{f}UₘU_{f}CₘC_{f}UₘG_{f}GₘU_{f}CₘU_{f}CₘU_{f}GₘU_{f}Uₘ-5′ | |
| 4/344178 s | 5′-AAGUAAGGACCAGAGACAAA-3′ | 1.41 |
| 5/303912 as | 3′-UUCAUUCCUGGUCUCUGUUA-5′ | |
| 1/366667 s | 5′-Chol-A_{f}AₘG_{f}UₘA_{f}AₘG_{f}GₘA_{f}CₘC_{f}AₘG_{f}AₘG_{f}AₘC_{f}AₘA_{f}-3′ | 0.077 |
| 2/352820 as | 3′-UₘU_{f}CₘA_{f}UₘU_{f}CₘC_{f}UₘG_{f}GₘU_{f}CₘU_{f}CₘU_{f}GₘU_{f}Uₘ-P-5′ | |
| 1/359996 s | 5′-A_{f}AₘG_{f}UₘA_{f}AₘG_{f}GₘA_{f}CₘC_{f}AₘG_{f}AₘG_{f}AₘC_{f}AₘA_{f}-3′ | 0.262 |
| 2/366561 as | 3′-Chol-UₘU_{f}CₘA_{f}UₘU_{f}CₘC_{f}UₘG_{f}GₘU_{f}CₘU_{f}CₘU_{f}GₘU_{f}Uₘ-5′ | |
| 1/366667 s | 5′-Chol-A_{f}AₘG_{f}UₘA_{f}AₘG_{f}GₘA_{f}CₘC_{f}AₘG_{f}AₘG_{f}AₘC_{f}AₘA_{f}-3′ | 0.229 |
| 2/359995 as | 3′-UₘU_{f}CₘA_{f}UₘU_{f}CₘC_{f}UₘG_{f}GₘU_{f}CₘU_{f}CₘU_{f}GₘU_{f}Uₘ-5′ | |

s = sense strand, as = antisense strand, ss = single strand.

### Example 18

### Synthesis of Pyrrolidine CPG Support

In some aspects of the invention the solid support is attached to the pyrrolidinyl group prior to the addition of the conjugate group as per the following scheme:

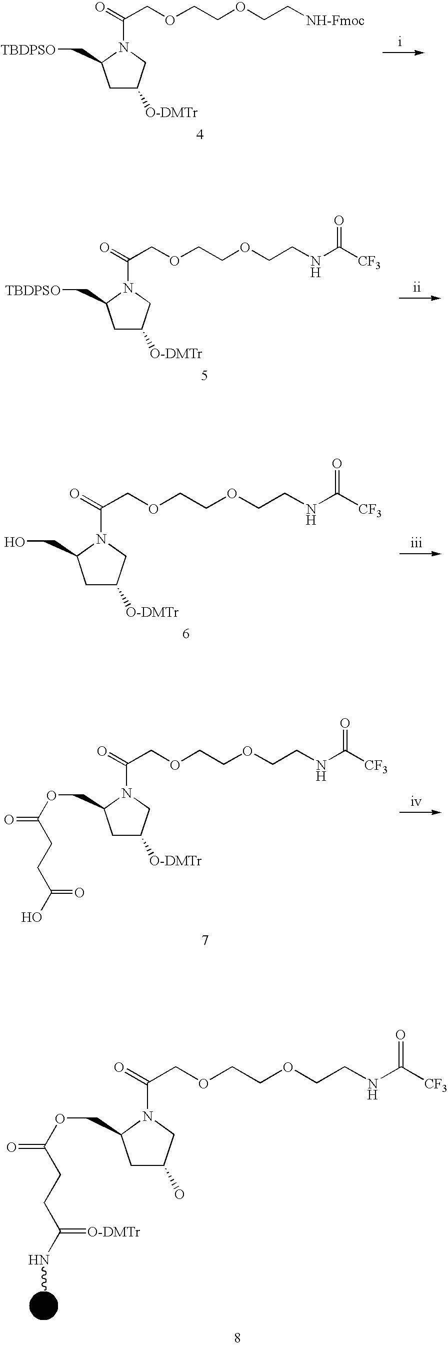

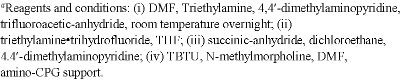

Compound 5 was prepared starting with compound 4 (1 g) which was dissolved in N′N-dimethylformamide (DMF, 10 mL) containing triethylamine (1 mL) and 4,4′-dimethylaminopyridine (100 mg) with stirring under a nitrogen atmosphere for 4 hrs. Trifluoroacetic-anhydride (0.4 mL) was added and the reaction was stirred overnight at room temperature. The reaction was quenched by poring onto ice (50 mL) and partitioned between ethyl acetate (50 mL) and a saturated sodium bicarbonate solution (100 mL). The ethyl acetate layer was washed with brine (10 mL) and dried over anhydrous sodium sulfate. The solvents were removed under reduced pressure and the residue (Compound 5) was used without further purification in the next step.

Compound 5 from the previous step was dissolved in anhydrous tetrahydrofuran (10 mL) and stirred under a nitrogen atmosphere. Triethylamine (0.5 mL) and triethylaminetrihydrofluoride (1 mL) were added and the reaction was stirred at room temperature overnight. The reaction mixture was partitioned between ethyl acetate (50 mL) and a saturated sodium bicarbonate solution (100 mL). The ethyl acetate layer was washed with brine (10 mL) and dried over anhydrous sodium sulfate. The solvents were removed under reduced pressure and the residue was purified by silica gel flash chromatography (10% methanol in methylene chloride) to give Compound 6 (420 mg, yield=65% over two steps). Electrospray MS: mass calc. for C₃₄H₃₉F₃N₂O₈=660.27, found=661.14 [M+H]⁺.

Compound 6 (400 mg) was dissolved in anhydrous 1,2-dichloroethane. 4,4′-dimethylaminopyridine (20 mg), and triethylamine (500 μL) were added followed by succinic anhydride (100 mg). The reaction was stirred under a nitrogen atmosphere overnight and then partitioned between ethyl acetate (20 mL) and ice-cold water (50 mL). The ethyl acetate layer was washed with ice-cold 10% citric acid solution (10 mL), brine (10 mL), and dried over anhydrous sodium sulfate. The solvents were evaporated under reduced pressure to give Compound 7 as a white powder which was dried overnight under high vacuum. This material needed no further purification and was used as such in the next step. Electrospray MS: mass calc. for C₃₈H₄₃F₃N₂O₁₁, found=761.28 [M+H]⁺.

Compound 7 (400 mg), TBTU (200 mg), and N-methylmorpholine (120 μL) were dissolved in dry DMF (6 mL). Controlled pore glass (CPG) (500 mg, pore size=500° A, loading=125 μM/g) was added and the mixture shaken for 18 hours using a mechanical shaker. The CPG was then transferred to a filtration funnel and washed with dry DMF (4×40 mL) and then with dry diethyl ether (2×40 mL). The CPG was then transferred to a 10 mL round bottom flask and treated with a 1:1 mix of Cap mix A:Cap mix B for 2 hrs. The CPG was then transferred to a filtration funnel and washed with dry DMF (4×40 mL) and then with dry diethyl ether (2×40 mL) to give the pyridinyl functionalized support medium 8. The degree of loading was determined by DMT-cleavage assay (treating the CPG with 3% trichloroacetic acid in dichloromethane) and measuring the absorbance at 502-nM. The loading was determined to be 48 μM/g.

### Example 19

### Synthesis of 3′-Amine Bearing Oligonucleotides

Oligonucleotides were synthesized on a solid phase DNA/RNA synthesizer using the 2′-O-TBS RNA phosphoramidites (TBS=tert-butyl dimethyl-silyl) according to the reported protocols. 2′-F, 2′-O-Me, and 2′-O-MOE phosphoramidites with exocyclic amino groups protected with benzoyl (Bz for A and C) protecting groups were used for the synthesis of the RNA chimera. 0.12 M solution of the phosphoramidites in anhydrous acetonitrile was used for the synthesis. 5′-Fluorescein phosphoramidite from Glen Research Corp. was used to synthesize 5′-fluorescein-labelled oligonucleotides. Oxidation of the internucleosidic phosphite to the phosphate was carried out using tert-butyl hydroperoxide/acetonitrile/water (10:87:3) with a 10 min oxidation time. 3-H-1,2-benzodithiol-3-one 1,1-dioxide (the Beaucage reagent, 0.15 M solution in anhydrous acetonitrile) was used as the sulfur-transfer agent for the synthesis of oligoribonucleotide phosphorothioates. Twelve equivalents of phosphoramidite solutions were delivered in two portions, each followed by a 6 min coupling wait time. All other steps in the protocol supplied by the manufacturer were used without modification. The step-wise coupling efficiencies were more than 97%. After completion of the synthesis, solid support was suspended in aqueous ammonium hydroxide (30 wt. %): ethanol (3:1) and heated at 55° C. for 6 h to complete the removal of all protecting groups except TBS group at 2′-position. The solid support was filtered and the filtrate was concentrated to dryness. The residue obtained was re-suspended in anhydrous triethylamine trihydrofluoride/triethylamine/1-methyl-2-pyrrolidinone solution (0.75 mL of a solution of 1 ml of triethylamine-trihydrofluoride, 750 μl triethylamine and 1.5 mL 1-methyl-2-pyrrolidine, to provide a 1.4 M HF concentration) and heated at 65° C. for 1.5 h to remove the TBDMS groups at the 2′-position. The reaction was quenched with 1.5 M ammonium bicarbonate (0.75 mL) and the mixture was loaded on to a Sephadex G-25 column (NAP Columns, Amersham Biosciences Inc.).

The oligonucleotides were eluted with water and the fractions containing the oligonucleotides were pooled together and purified by HPLC on a strong anion exchange column (Mono Q, Pharmacia Biotech, 16/10, 20 mL, 10 μm, ionic capacity 0.27-0.37 mmole/mL, A=100 mM ammonium acetate, 30% aqueous acetonitrile, B=1.5 M NaBr in A, 0 to 60% B in 40 min, Flow 1.5 mL min⁻¹, λ=260 nm). Fractions containing full-length oligonucleotides were pooled together (assessed by CGE analysis >90%) and evaporated. The residue was dissolved in sterile water (0.3 mL) and absolute ethanol (1 mL) was added and cooled in dry ice (−20° C.) for 1 h and the precipitate formed was pelleted out by centrifugation (NYCentrifuge 5415C; Eppendorf, Westbury, N.Y.) at 3000 rpm. The supernatant was decanted and the pellet was re-dissolved in 10 M ammonium acetate (0.3 mL) solution. Ethanol (1 mL) was added and cooled to −20° C. for 1 h to get a precipitate which was pelleted out in a centrifuge (NYCentrifuge 5415C; Eppendorf, Westbury, N.Y.) at 3000-rpm for 15 min. The pellet was collected by decanting the supernatant. The pellet was re-dissolved in sterile water (0.3 mL) and precipitated by adding ethanol (1 mL) and cooling the mixture at −20° C. for 1 h. The precipitate formed was pelleted out and collected as described above. The oligonucleotides were characterized by ES MS and purity was assessed by capillary gel electrophoresis.

Following this procedure

### Oligonucleotide Sequences Synthesized:

| SEQ ID NO./ | |
|---|---|
| ISIS NO. | Sequence |
| 6/371313 | 5′-GₑGₑAₑGAUCAACAUUUCAₑAₑAₑ- |
| | pyrrolidine-NH₂-3′ |
| 6/355714 | 5′-G_{f}GₘA_{f}GₘA_{f}UₘC_{f}AₘA_{f}CₘA_{f}UₘU_{f}UₘU_{f}CₘA_{f}Aₘ |
| | A_{f}-pyrrolidine-NH₂-3′ |

5′-Fluoresceinated versions of these oligonucleotides were also synthesized.

### Example 20

### Coupling of mPEG (20K)-NHS Esters to 3′-Mino-Bearing Oligomeric Compounds

Oligomeric compounds having a 3′-free amino moiety attached through a pyrrolidinyl group were further functionalized with mPEG groups as per the following scheme:

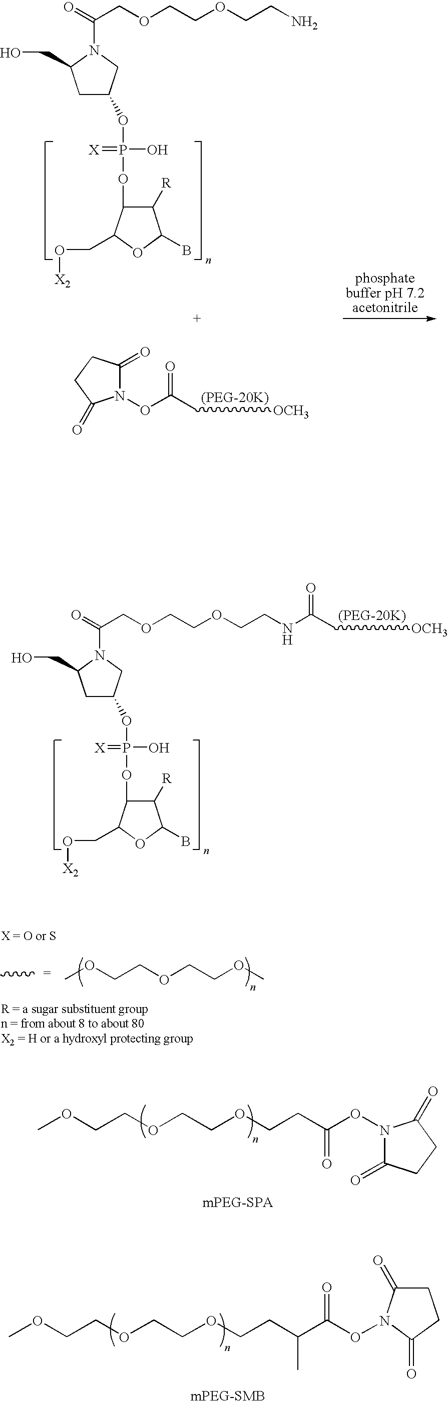

Initial studies indicated that a pH from about 7.2 to about 7.4 is the ideal pH for coupling between the MPEG-NHS (NHS=n-Hydroxysuccinimidyl) ester and the amine bearing oligonucleotide. At lower pHs the rate of reaction decreased appreciably while at higher pH values (pH 8.0 and above) the rate of aqueous hydrolysis of the PEG-MHS ester increased dramatically and decreased no productive coupling was observed.

Two different NHS esters were evaluated, MPEG-SPA (mPEG-Succinimidyl Propionate) and MPEG-SMB (Succinimidyl α-methylbutanoate). mPEG-SMB is slower reacting than the mPEG-SPA but is more selective for aliphatic amine coupling and has a longer half-life during the coupling reaction.

General Procedures: oligonucleotide (100 O.Ds in 2 mL final volume) was dissolved in a 1:1 mixture of 0.1 M phosphate buffer pH 7.2 and acetonitrile. polyethylene glycol (20,000 mol. Wt.) monomethyl ether n-hydroxysuccinimidyl ester (30 equivalents) was added in two batches at 10 hr intervals. After shaking for 20 hrs on a mechanical shaker the reaction mixture was transferred to a Labconco centrivap and concentrated to dryness. The residue was dissolved in 15 mL DNAse/RNAse free water (15 mL) and purified by ion exchange chromatography (Mono Q, Pharmacia Biotech, 16/10, 20 mL, 10 μm, ionic capacity 0.27-0.37 mmole/mL, A=100 mM ammonium acetate, 30% aqueous acetonitrile, B=1.5 M NaBr in A, 0 to 60% B in 40 min, Flow 1.5 mL min⁻¹, λ=260 nm) followed by desalting using reverse phase HPLC (Phenomenex Jupiter, 10×250 MM, water/acetonitrile gradient, flow=5 mL/min, λ=260 nm). The desired fractions were concentrated and analyzed by reverse-phase HPLC and MALDI mass spectrometry.

### Example 21

### Synthesis of C-16 Conjugated Pyrrolidinyl Amidite Group

C-16 conjugated to a pyrrolidinyl amidite group was prepared as per the following scheme:

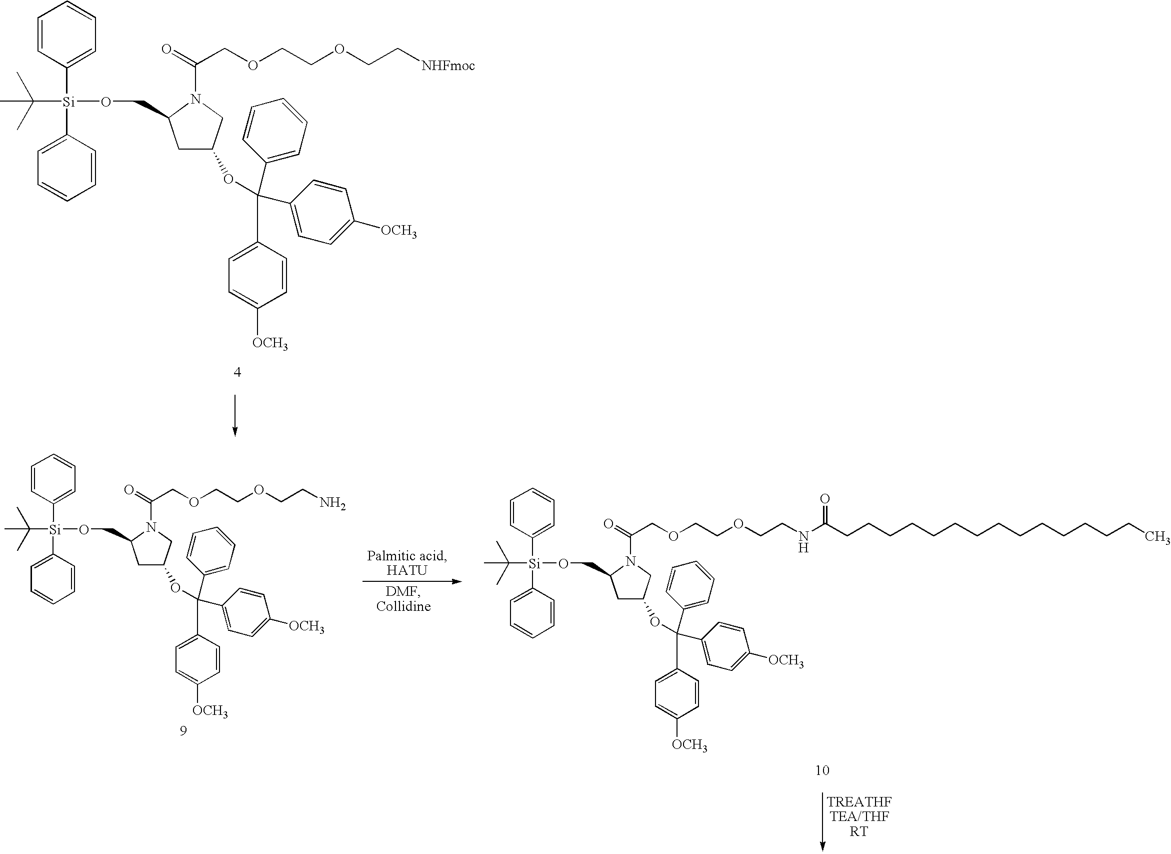

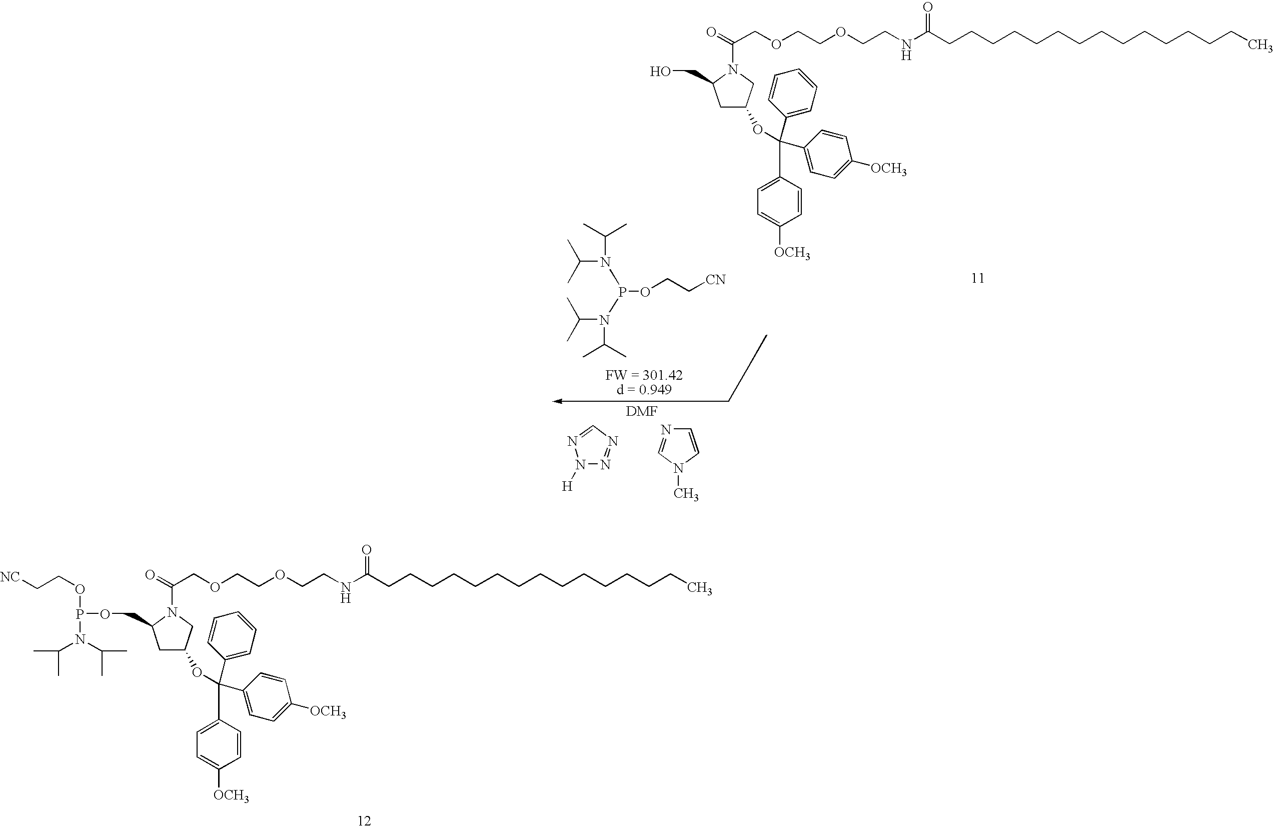

Compound 4 (1.5 g) was dissolved in N′N-dimethylformamide (DMF, 5 mL). To this was added piperidine (0.37 g, 3 equivalents) with stirring under a nitrogen atmosphere for 2 hrs. The reaction as observed by TLC was complete. The reaction mixture was diluted with ethyl acetate (50 mL), washed with water (5×20 mL), washed with brine (10 mL) and dried over anhydrous sodium sulfate. The solvents were removed under reduced pressure and the residue was purified on a short silica gel column using 20% MeOH in CH₂Cl₂ containing 0.5% Et₃N. Appropriate fractions were concentrated to an oil to give Compound 9 (1.1 g). The structure was confirmed by 1HNMR and ESMS.

Palmitic acid (360 mg) was dissolved in anhydrous DMF (5 mL) with cooling to 0° C. and HATU (360 mg, one portion) was added. The reaction mixture was allowed to stir for 10 minutes. DIPEA (490 uL) was added followed by Compound 9 (0.96 g). The reaction mixture was stirred for 3 h. TLC (5% MeOH/CH₂Cl₂) indicated the reaction was complete. The reaction mixture was dissolved in EtOAc (30 mL) and washed with water (3×30 mL). The organic layer was dried over anhydrous Na₂SO₄ and concentrated to an oil which was purified on silica gel using 5% MeOH/CH₂Cl₂ as eluant. Appropriate fractions were collected and concentrated to glassy foam which was further dried under vacuum to give Compound 10 (70% yield). The structure was confirmed by 1H NMR and ESMS.

Compound 10 (0.8 g) was dissolved in anhydrous tetrahydrofuran (15 mL) and stirred under a nitrogen atmosphere. Triethylamine (0.8 mL) and triethylaminetrihydrofluoride (1 mL) were added and the reaction was stirred at room temperature overnight. The reaction mixture was partitioned between ethyl acetate (50 mL) and a saturated sodium bicarbonate solution (100 mL). The ethyl acetate layer was washed with brine (10 mL) and dried over anhydrous sodium sulfate. The solvents were removed under reduced pressure and the residue was purified by silica gel flash chromatography (10% methanol in methylene chloride) to give Compound II (0.6 g). The Structure was confirmed by 1H NMR and mass spectral analysis.

Compound II (0.295 g) was mixed with 1-H tetrazole (21 mg, 0.8 equivalents1) and dried over P₂O₅ in vacuum overnight at 40° C. The mixture was dissolved in anhydrous DMF (2 mL) and 2-cyanoethyl N,N,N′N′-tetraisopropylphosphorodiamidite (180 μL, 1.5 equivalents) was added drop-wise followed by the addition of N-methylimidazole (7 uL, 0.25 equivalents). The reaction mixture was stirred at room temperature under an argon atmosphere for 8 h. The solvent was removed under reduced pressure. The residue was purified by silica gel flash column chromatography (20% ethyl acetate in hexane) to give Compound 12 (195 mg as an oil). The structure was confirmed by 1H NMR and 31P NMR.

### Example 22

### Synthesis of C16-Conjugated Oligomeric Compounds

The C-16 conjugated oligomeric compounds illustrated below were prepared with the C-16 conjugate attached to either the 5′ or 3′ end

| SEQ ID NO./ | |
|---|---|
| ISIS NO. | Sequence |
| 6/388455 | 5′-GₑGₑAₑGAUCAACAUUUUCAₑAₑAₑ-C16-3′ |
| 6/388456 | 5′-GₑGₑAₑGAUCAACAUUUUCAₑAₑAₑ-C16-3′ |
| 6/388457 | 5′-(6-carboxyfluorescein)-GₑGₑAₑGAUCAAC |
| | AUUUUCAₑAₑAₑs-C16-3′ |
| 6/388458 | 5′-C16-sGₑGₑAₑGAUCAACAUUUUCAₑAₑAₑ-3′ |

C16 alkyl chain is conjugated through a pyrrolidine linker, the phosphoramidite of which was synthesized as described as above. This single phosphoramidite can be used to modify either the 5′ or 3′ termini of the oligonucleotide.

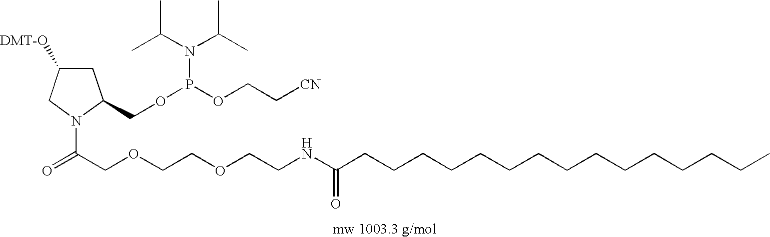

### Synthesis of oligonucleotides on a 2 μmol scale was carried out on an ABI 394 DNA/RNA synthesizer. All phosphoramidites, including the C16-pyrrolidine phosphoramidite, were prepared as 0.1 M solutions in acetonitrile. Incorporation of phosphorothioates was achieved by thiolation with a 0.15 M solution of Beaucage reagent in acetonitrile, while oxidation of phosphodiesters was achieved using 10% tert-butylhydroperoxide in wet acetonitrile. All other reagents were standard.

All oligonucleotides were synthesized using UnyLinker CPG (loading of 48 μmol/gram). The initial dimethoxytrityl group was removed from the UnyLinker CPG using a 45 second treatment with 3% (w/v) trichloroacetic acid in dichloromethane. Other than doubling the length of the detritylation step, the C16-pyrrolidine phosphoramidite was coupled using standard RNA coupling conditions (double-delivery of phosphoramidite with a total coupling time of 12 minutes).

Following synthesis, each CPG-bound oligonucleotide was treated first with 1:1 triethylamine:acetonitrile for 1 hour at room temperature, then with 3:1 aqueous ammonium hydroxide:ethanol at 55° C. for 8 hours. Following ammonia deprotection, samples were dried and treated for two hours at 65° C. with 0.4 mL of a mixture of 1.5 mL N-methylpyrrolidinone, 0.75 mL triethylamine, and 1.0 mL triethylammonium-trihydrofluoride to facilitate removal of the 2′-O-tertbutyldimethylsilyl protecting groups. After desalting, unpurified oligonucleotides were analyzed by ESI-MS to confirm the presence of the correct product. The results of this analysis are presented below:

| SEQ ID NO. Sequence/ | Molecular Weight | |
|---|---|---|
| ISIS NO. | Da Calcd. | Observed |
| 6/388455 | 6964.8 | 6964.3 |
| 6/388456 | 6980.9 | 6980.2 |
| 6/388457 | 7548.4 | 7547.8 |
| 6/388458 | 6980.9 | 6980.4 |

Constructs 388455, 388456, 388457 and 388458 were purified by methods previously disclosed.

### Example 23

### Oligonucleotide Isolation

After cleavage from the controlled pore glass solid support and deblocking in concentrated ammonium hydroxide at 55° C. for 12-16 hours, the oligonucleotides or oligonucleosides are recovered by precipitation out of 1 M NH₄OAc with >3 volumes of ethanol. Synthesized oligonucleotides were analyzed by electrospray mass spectroscopy (molecular weight determination) and by capillary gel electrophoresis and judged to be at least 70% full length material. The relative amounts of phosphorothioate and phosphodiester linkages obtained in the synthesis was determined by the ratio of correct molecular weight relative to the −16 amu product (+/−32+/−48). For some studies oligonucleotides were purified by HPLC, as described by Chiang et al., J. Biol. Chem. 1991, 266, 18162-18171. Results obtained with HPLC-purified material were similar to those obtained with non-HPLC purified material.

### Example 24

### Oligonucleotide Synthesis

### 96 Well Plate Format

Oligonucleotides are synthesized via solid phase P(III) phosphoramidite chemistry on an automated synthesizer capable of assembling 96 sequences simultaneously in a 96-well format. Phosphodiester internucleotide linkages are afforded by oxidation with aqueous iodine. Phosphorothioate internucleotide linkages are generated by sulfinurization utilizing 3,H-1,2 benzodithiole-3-one 1,1 dioxide (Beaucage Reagent) in anhydrous acetonitrile. Standard base-protected beta-cyanoethyl-diiso-propyl phosphoramidites are purchased from commercial vendors (e.g. PE-Applied Biosystems, Foster City, Calif., or Pharmacia, Piscataway, N.J.). Non-standard nucleosides are synthesized as per standard or patented methods. They are utilized as base protected beta-cyanoethyldiisopropyl phosphoramidites.

Oligonucleotides are cleaved from support and deprotected with concentrated NH₄OH at elevated temperature (55-60° C.) for 12-16 hours and the released product then dried in vacuo. The dried product is then re-suspended in sterile water to afford a master plate from which all analytical and test plate samples are then diluted utilizing robotic pipettors.

### Example 25

### Cell Culture and Oligonucleotide Treatment

The effect of oligomeric compounds on target nucleic acid expression can be tested in any of a variety of cell types provided that the target nucleic acid is present at measurable levels. This can be routinely determined using, for example, PCR or Northern blot analysis. The following cell types are provided for illustrative purposes, but other cell types can be routinely used, provided that the target is expressed in the cell type chosen. This can be readily determined by methods routine in the art, for example Northern blot analysis, ribonuclease protection assays, or RT-PCR.

### T-24 Cells:

The human transitional cell bladder carcinoma cell line T-24 was obtained from the American Type Culture Collection (ATCC) (Manassas, Va.). T-24 cells were routinely cultured in complete McCoy's 5A basal media (Invitrogen Corporation, Carlsbad, Calif.) supplemented with 10% fetal calf serum (Invitrogen Corporation, Carlsbad, Calif.), penicillin 100 units per mL, and streptomycin 100 micrograms per mL (Invitrogen Corporation, Carlsbad, Calif.). Cells were routinely passaged by trypsinization and dilution when they reached 90% confluence. Cells were seeded into 96-well plates (Falcon-Primaria #353872) at a density of 7000 cells/well for use in RT-PCR analysis.

For Northern blotting or other analysis, cells may be seeded onto 100 mm or other standard tissue culture plates and treated similarly, using appropriate volumes of medium and oligonucleotide.

### A549 Cells:

The human lung carcinoma cell line A549 was obtained from the American Type Culture Collection (ATCC) (Manassas, Va.). A549 cells were routinely cultured in DMEM basal media (Invitrogen Corporation, Carlsbad, Calif.) supplemented with 10% fetal calf serum (Invitrogen Corporation, Carlsbad, Calif.), penicillin 100 units per mL, and streptomycin 100 micrograms per mL (Invitrogen Corporation, Carlsbad, Calif.). Cells were routinely passaged by trypsinization and dilution when they reached 90% confluence.

### NHDF Cells:

Human neonatal dermal fibroblast (NHDF) were obtained from the Clonetics Corporation (Walkersville, Md.). NHDFs were routinely maintained in Fibroblast Growth Medium (Clonetics Corporation, Walkersville, Md.) supplemented as recommended by the supplier. Cells were maintained for up to 10 passages as recommended by the supplier.

### HEK Cells:

Human embryonic keratinocytes (HEK) were obtained from the Clonetics Corporation (Walkersville, Md.). HEKs were routinely maintained in Keratinocyte Growth Medium (Clonetics Corporation, Walkersville, Md.) formulated as recommended by the supplier. Cells were routinely maintained for up to 10 passages as recommended by the supplier.

Treatment with Oligomeric Compounds:

When cells reached 65-75% confluency, they were treated with oligonucleotide. For cells grown in 96-well plates, wells were washed once with 100 μL OPTI-MEM™-1 reduced-serum medium (Invitrogen Corporation, Carlsbad, Calif.) and then treated with 130 μL of OPTI-MEM™-1 containing 3.75 μg/mL LIPOFECTIN™ (Invitrogen Corporation, Carlsbad, Calif.) and the desired concentration of oligonucleotide. Cells are treated and data are obtained in triplicate. After 4-7 hours of treatment at 37° C., the medium was replaced with fresh medium. Cells were harvested 16-24 hours after oligonucleotide treatment.

The concentration of oligonucleotide used varies from cell line to cell line. To determine the optimal oligonucleotide concentration for a particular cell line, the cells are treated with a positive control oligonucleotide at a range of concentrations. For human cells the positive control oligonucleotide is selected from either ISIS 13920 (TₑCₑCₑGTCATCGCTCₑCₑTₑCₑAₑGₑGₑGₑ, SEQ ID NO: 7) which is targeted to human H-ras, or ISIS 18078, (GₑTₑGₑCₑGₑCGCGAGCCCGₑAₑAₑAₑTₑCₑ, SEQ ID NO: 8) which is targeted to human Jun-N-terminal kinase-2 (JNK2). Both controls are 2′-O-methoxyethyl gapmers (2′-O-methoxyethyls shown in bold) with a phosphorothioate backbone. For mouse or rat cells the positive control oligonucleotide is ISIS 15770, AₑTₑGₑCₑAₑTTCTGCCCCCAₑAₑGₑGₑAₑ, SEQ ID NO: 9, a 2′-O-methoxyethyl gapmer (2′-O-methoxyethyls shown in bold) with a phosphorothioate backbone which is targeted to both mouse and rat c-raf. The concentration of positive control oligonucleotide that results in 80% inhibition of c-H-ras (for ISIS 13920), JNK2 (for ISIS 18078) or c-raf (for ISIS 15770) mRNA is then utilized as the screening concentration for new oligonucleotides in subsequent experiments for that cell line. If 80% inhibition is not achieved, the lowest concentration of positive control oligonucleotide that results in 60% inhibition of c-H-ras, JNK2 or c-raf mRNA is then utilized as the oligonucleotide screening concentration in subsequent experiments for that cell line. If 60% inhibition is not achieved, that particular cell line is deemed as unsuitable for oligonucleotide transfection experiments. The concentrations of antisense oligonucleotides used herein are from 50 nM to 300 nM.

### Example 26

### Analysis of Oligonucleotide Inhibition of a Target Expression

Antisense modulation of a target expression can be assayed in a variety of ways known in the art. For example, a target mRNA levels can be quantitated by, e.g., Northern blot analysis, competitive polymerase chain reaction (PCR), or real-time PCR(RT-PCR). Real-time quantitative PCR is presently preferred. RNA analysis can be performed on total cellular RNA or poly(A)+ mRNA. The preferred method of RNA analysis of the present invention is the use of total cellular RNA as described in other examples herein. Methods of RNA isolation are well known in the art. Northern blot analysis is also routine in the art. Real-time quantitative (PCR) can be conveniently accomplished using the commercially available ABI PRISM™ 7600, 7700, or 7900 Sequence Detection System, available from PE-Applied Biosystems, Foster City, Calif. and used according to manufacturer's instructions.

Protein levels of a target can be quantitated in a variety of ways well known in the art, such as immunoprecipitation, Western blot analysis (immunoblotting), enzyme-linked immunosorbent assay (ELISA) or fluorescence-activated cell sorting (FACS). Antibodies directed to a target can be identified and obtained from a variety of sources, such as the MSRS catalog of antibodies (Aerie Corporation, Birmingharn, Mich.), or can be prepared via conventional monoclonal or polyclonal antibody generation methods well known in the art.

### Example 27

### RNA Isolation

Poly(A)+ mRNA Isolation

Poly(A)+ mRNA was isolated according to Miura et al., (Clin. Chem., 1996, 42, 1758-1764). Other methods for poly(A)+ mRNA isolation are routine in the art. Briefly, for cells grown on 96-well plates, growth medium was removed from the cells and each well was washed with 200 μL cold PBS. 60 μL lysis buffer (10 mM Tris-HCl, pH 7.6, 1 mM EDTA, 0.5 M NaCl, 0.5% NP-40, 20 mM vanadyl-ribonucleoside, complex) was added to each well, the plate was gently agitated and then incubated at room temperature for five minutes. 55 μL of lysate was transferred to Oligo d(T) coated 96-well plates (AGCT Inc., Irvine Calif.). Plates were incubated for 60 minutes at room temperature, washed 3 times with 200 μL of wash buffer (10 mM Tris-HCl pH 7.6, 1 mM EDTA, 0.3 M NaCl). After the final wash, the plate was blotted on paper towels to remove excess wash buffer and then air-dried for 5 minutes. 60 μL of elution buffer (5 mM Tris-HCl pH 7.6), preheated to 70° C., was added to each well, the plate was incubated on a 90° C. hot plate for 5 minutes, and the eluate was then transferred to a fresh 96-well plate.

Cells grown on 100 mm or other standard plates may be treated similarly, using appropriate volumes of all solutions.

### Total RNA Isolation

Total RNA was isolated using an RNEASY 96™ kit and buffers purchased from Qiagen Inc. (Valencia, Calif.) following the manufacturer's recommended procedures. Briefly, for cells grown on 96-well plates, growth medium was removed from the cells and each well was washed with 200 μL cold PBS. 150 μL Buffer RLT was added to each well and the plate vigorously agitated for 20 seconds. 150 μL of 70% ethanol was then added to each well and the contents mixed by pipetting three times up and down. The samples were then transferred to the RNEASY 96™ well plate attached to a QIAVAC™ manifold fitted with a waste collection tray and attached to a vacuum source. Vacuum was applied for 1 minute. 500 μL of Buffer RW1 was added to each well of the RNEASY 96™ plate and incubated for 15 minutes and the vacuum was again applied for 1 minute. An additional 500 μL of Buffer RW1 was added to each well of the RNEASY 96™ plate and the vacuum was applied for 2 minutes. 1 mL of Buffer RPE was then added to each well of the RNEASY 96™ plate and the vacuum applied for a period of 90 seconds. The Buffer RPE wash was then repeated and the vacuum was applied for an additional 3 minutes. The plate was then removed from the QIAVAC™ manifold and blotted dry on paper towels. The plate was then re-attached to the QIAVAC™ manifold fitted with a collection tube rack containing 1.2 mL collection tubes. RNA was then eluted by pipetting 140 μL of RNAse free water into each well, incubating 1 minute, and then applying the vacuum for 3 minutes.

The repetitive pipetting and elution steps may be automated using a QIAGEN Bio-Robot 9604 (Qiagen, Inc., Valencia Calif.). Essentially, after lysing of the cells on the culture plate, the plate is transferred to the robot deck where the pipetting, DNase treatment and elution steps are carried out.

### Example 28

### Real-Time Quantitative PCR Analysis of a Target mRNA Levels

Quantitation of a target mRNA levels was accomplished by real-time quantitative PCR using the ABI PRISM™ 7600, 7700, or 7900 Sequence Detection System (PE-Applied Biosystems, Foster City, Calif.) according to manufacturer's instructions. This is a closed-tube, non-gel-based, fluorescence detection system which allows high-throughput quantitation of polymerase chain reaction (PCR) products in real-time. As opposed to standard PCR in which amplification products are quantitated after the PCR is completed, products in real-time quantitative PCR are quantitated as they accumulate. This is accomplished by including in the PCR reaction an oligonucleotide probe that anneals specifically between the forward and reverse PCR primers, and contains two fluorescent dyes. A reporter dye (e.g., FAM or JOE, obtained from either PE-Applied Biosystems, Foster City, Calif., Operon Technologies Inc., Alameda, Calif. or Integrated DNA Technologies Inc., Coralville, Iowa) is attached to the 5′ end of the probe and a quencher dye (e.g., TAMRA, obtained from either PE-Applied Biosystems, Foster City, Calif., Operon Technologies Inc., Alameda, Calif. or Integrated DNA Technologies Inc., Coralville, Iowa) is attached to the 3′ end of the probe. When the probe and dyes are intact, reporter dye emission is quenched by the proximity of the 3′ quencher dye. During amplification, annealing of the probe to the target sequence creates a substrate that can be cleaved by the 5′-exonuclease activity of Taq polymerase. During the extension phase of the PCR amplification cycle, cleavage of the probe by Taq polymerase releases the reporter dye from the remainder of the probe (and hence from the quencher moiety) and a sequence-specific fluorescent signal is generated. With each cycle, additional reporter dye molecules are cleaved from their respective probes, and the fluorescence intensity is monitored at regular intervals by laser optics built into the ABI PRISM™ Sequence Detection System. In each assay, a series of parallel reactions containing serial dilutions of mRNA from untreated control samples generates a standard curve that is used to quantitate the percent inhibition after antisense oligonucleotide treatment of test samples.

Prior to quantitative PCR analysis, primer-probe sets specific to the target gene being measured are evaluated for their ability to be “multiplexed” with a GAPDH amplification reaction. In multiplexing, both the target gene and the internal standard gene GAPDH are amplified concurrently in a single sample. In this analysis, mRNA isolated from untreated cells is serially diluted. Each dilution is amplified in the presence of primer-probe sets specific for GAPDH only, target gene only (“single-plexing”), or both (multiplexing). Following PCR amplification, standard curves of GAPDH and target mRNA signal as a function of dilution are generated from both the single-plexed and multiplexed samples. If both the slope and correlation coefficient of the GAPDH and target signals generated from the multiplexed samples fall within 10% of their corresponding values generated from the single-plexed samples, the primer-probe set specific for that target is deemed multiplexable. Other methods of PCR are also known in the art.

PCR reagents were obtained from Invitrogen Corporation, (Carlsbad, Calif.). RT-PCR reactions were carried out by adding 20 μL PCR cocktail (2.5×PCR buffer minus MgCl₂, 6.6 mM MgCl₂, 375 μM each of DATP, dCTP, dCTP and dGTP, 375 nM each of forward primer and reverse primer, 125 mM of probe, 4 Units RNAse inhibitor, 1.25 Units PLATINUM® Taq, 5 Units MuLV reverse transcriptase, and 2.5× ROX dye) to 96-well plates containing 30 μL total RNA solution (20-200 ng). The RT reaction was carried out by incubation for 30 minutes at 48° C. Following a 10 minute incubation at 95° C. to activate the PLATINUM® Taq, 40 cycles of a two-step PCR protocol were carried out: 95° C. for 15 seconds (denaturation) followed by 60° C. for 1.5 minutes (annealing/extension).

Gene target quantities obtained by real time RT-PCR are normalized using either the expression level of GAPDH, a gene whose expression is constant, or by quantifying total RNA using RiboGreen™ (Molecular Probes, Inc. Eugene, Oreg.). GAPDH expression is quantified by real time RT-PCR, by being run simultaneously with the target, multiplexing, or separately. Total RNA is quantified using RiboGreen™ RNA quantification reagent (Molecular Probes, Inc. Eugene, Oreg.). Methods of RNA quantification by RiboGreen™ are taught in Jones, L. J., et al, (Analytical Biochemistry, 1998, 265, 368-374).

In this assay, 170 μL of RiboGreen™ working reagent (RiboGreen™ reagent diluted 1:350 in 10 mM Tris-HCl, 1 mM EDTA, pH 7.5) is pipetted into a 96-well plate containing 30 μL purified, cellular RNA. The plate is read in a CytoFluor 4000 (PE Applied Biosystems) with excitation at 485 nm and emission at 530 nm.

Probes and are designed to hybridize to a human a target sequence, using published sequence information.

### Example 29

### Northern Blot Analysis of a Target mRNA Levels

Eighteen hours after antisense treatment, cell monolayers were washed twice with cold PBS and lysed in 1 mL RNAZOL™ (TEL-TEST “B” Inc., Friendswood, Tex.). Total RNA was prepared following manufacturer's recommended protocols. Twenty micrograms of total RNA was fractionated by electrophoresis through 1.2% agarose gels containing 1.1% formaldehyde using a MOPS buffer system (AMRESCO, Inc. Solon, Ohio). RNA was transferred from the gel to HYBOND™-N+ nylon membranes (Amersham Pharmacia Biotech, Piscataway, N.J.) by overnight capillary transfer using a Northern/Southern Transfer buffer system (TEL-TEST “B” Inc., Friendswood, Tex.). RNA transfer was confirmed by UV visualization. Membranes were fixed by UV cross-linking using a STRATALINKER™ UV Crosslinker 2400 (Stratagene, Inc, La Jolla, Calif.) and then probed using QUICKHYB™ hybridization solution (Stratagene, La Jolla, Calif.) using manufacturer's recommendations for stringent conditions.

To detect human a target, a human a target specific primer probe set is prepared by PCR To normalize for variations in loading and transfer efficiency membranes are stripped and probed for human glyceraldehyde-3-phosphate dehydrogenase (GAPDH) RNA (Clontech, Palo Alto, Calif.).

Hybridized membranes were visualized and quantitated using a PHOSPHORIMAGER™ and IMAGEQUANT™ Software V3.3 (Molecular Dynamics, Sunnyvale, Calif.). Data was normalized to GAPDH levels in untreated controls.

### Example 30

### Inhibition of Human a Target Expression by Oligomeric Compounds

In accordance with the present invention, a series of oligomeric compounds are designed to target different regions of the human target RNA. The oligomeric compounds are analyzed for their effect on human target mRNA levels by quantitative real-time PCR as described in other examples herein. Data are averages from three experiments. The target regions to which these preferred sequences are complementary are herein referred to as “preferred target segments” and are therefore preferred for targeting by oligomeric compounds of the present invention. The sequences represent the reverse complement of the preferred oligomeric compounds.

As these “preferred target segments” have been found by experimentation to be open to, and accessible for, hybridization with the oligomeric compounds of the present invention, one of skill in the art will recognize or be able to ascertain, using no more than routine experimentation, further embodiments of the invention that encompass other oligomeric compounds that specifically hybridize to these preferred target segments and consequently inhibit the expression of a target.

According to the present invention, oligomeric compounds include antisense oligomeric compounds, antisense oligonucleotides, ribozymes, external guide sequence (EGS) oligonucleotides, alternate splicers, primers, probes, and other short oligomeric compounds which hybridize to at least a portion of the target nucleic acid.

### Example 31

### Western Blot Analysis of Target Protein Levels

Western blot analysis (immunoblot analysis) is carried out using standard methods. Cells are harvested 16-20 h after oligonucleotide treatment, washed once with PBS, suspended in Laemmli buffer (100 ul/well), boiled for 5 minutes and loaded on a 16% SDS-PAGE gel. Gels are run for 1.5 hours at 150 V, and transferred to membrane for western blotting. Appropriate primary antibody directed to a target is used, with a radiolabeled or fluorescently labeled secondary antibody directed against the primary antibody species. Bands are visualized using a PHOSPHORIMAGER™ (Molecular Dynamics, Sunnyvale Calif.).

### Example 32

### Liposome-Mediated Treatment with Oligomeric Compounds of the Invention

When cells reach the desired confluency, they can be treated with the oligomeric compounds of the invention by liposome-mediated transfection. For cells grown in 96-well plates, wells are washed once with 200 μL OPTI-MEM™-1 reduced-serum medium (Gibco BRL) and then treated with 100 μL of OPTI-MEM™-1 containing 2.5 μg/mL LIPOFECTIN™ (Gibco BRL) and the oligomeric compounds of the invention at the desired final concentration. After 4 hours of treatment, the medium is replaced with fresh medium. Cells are harvested 16 hours after treatment with the oligomeric compounds of the invention for target mRNA expression analysis by real-time PCR.

### Example 33

### Electroporation-Mediated Treatment with Oligomeric Compounds of the Invention

When the cells reach the desired confluency, they can be treated with the oligomeric compounds of the invention by electroporation. Cells are electroporated in the presence of the desired concentration of an oligomeric compound of the invention in 1 mm cuvettes at a density of 1×10⁷ cells/mL, a voltage of 75V and a pulse length of 6 ms. Following the delivery of the electrical pulse, cells are replated for 16 to 24 hours. Cells are then harvested for target mRNA expression analysis by real-time PCR.

1. A compound having the formula: 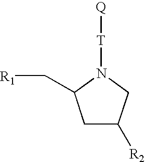
wherein:
R₁ is an activated phosphite group, X₁-Y or J-SM or when R₂ is X₂-Y then R₁ is hydroxyl, a protected hydroxyl an activated phosphite group, X₁-Y or J-SM;
J is a bivalent linking moiety;
SM is a support medium;
R₂ is hydroxyl, a protected hydroxyl or X₂-Y;
X₁ is an internucleoside linking group connecting a 5′-position of a nucleoside, nucleotide, an oligonucleoside, oligonucleotide or an oligomeric compound;
X₂ is an internucleoside linking group connecting a 3′-position of a nucleoside, nucleotide, an oligonucleoside, oligonucleotide or an oligomeric compound;
each Y is, independently, a nucleoside, nucleotide, an oligonucleoside, oligonucleotide or an oligomeric compound;
T is a bivalent tethering moiety; and
Q is a conjugate group.

2. The compound of claim 1 wherein R₁ is hydroxyl or a protected hydroxyl.

3. The compound of claim 2 wherein said protected hydroxyl comprises a protecting group selected from trityl, monomethoxytrityl, dimethoxytrityl, trimethoxytrityl, 9-phenylxanthin-9-yl (Pixyl) or 9-(p-methoxyphenyl)xanthin-9-yl (MOX).

4. The compound of claim 1 wherein R₁ is J-SM.

5. The compound of claim 4 wherein J-SM has the formula:
C(═O)—R₄—C(═O)—SM
wherein
R₄ is C₁-C₁₂ alkyl or substituted C₁-C₁₂ alkyl, wherein said alkyl group can be interrupted by one or more heteroatoms selected from N(Rₐ), S and O;
Rₐ is H, C₁-C₁₂ alkyl or substituted C₁-C₁₂ alkyl; and SM is a support medium.

6. The compound of claim 5 wherein R₄ is C₁-C₁₂ alkyl.

7. The compound of claim 6 wherein R₄ is CH₂CH₂.

8. The compound of claim 1 wherein R₁ is X₁-Y.

9. The compound of claim 8 wherein X₁ is phosphodiester, phosphorothioate or chiral phosphorothioate.

10. The compound of claim 9 wherein Y is an oligomeric compound.

11. The compound of claim 4 wherein SM is aminoalkyl controlled pore glass (CPG).

12. The compound of claim 1 wherein R₂ is hydroxyl or a protected hydroxyl.

13. The compound of claim 1 wherein R₂ is X₂-Y.

14. The compound of claim 13 wherein X₂ is a phosphodiester, phosphorothioate or a chiral phosphorothioate.

15. The compound of claim 14 wherein Y is an oligomeric compound.

16. The compound of claim 1 wherein said bivalent tethering moiety T has the formula:
*-C(═O)-E-N(Rₐ)—
wherein
* is attached to the N atom of the pyrrolidinyl group; and
E is a C₁-C₁₂ alkyl or substituted C₁-C₁₂ alkyl, wherein said alkyl groups are optionally further interrupted with from 1 to 5 heteroatoms selected from O, S or N(Rₐ), said substituent groups are selected from ═O and N(Rₐ).
each Rₐ is, independently, H or C₁-C₁₂ alkyl.

17. The compound of claim 16 wherein said bivalent tethering moiety is selected from —C(═O)—CH₂—O—(CH₂)₂—O—(CH₂)₂—N(Rₐ)— and —C(═O)—(CH₂)₅—N(Rₐ)—.

18. The compound of claim 1 wherein said activated phosphite moiety comprises a phosphoramidite, H-phosphonate, phosphate triester or a chiral auxiliary.

19. The compound of claim 1 having at least one Y group and wherein said at least one Y group is an oligomeric compound.

20. An oligomeric compound having the formula: 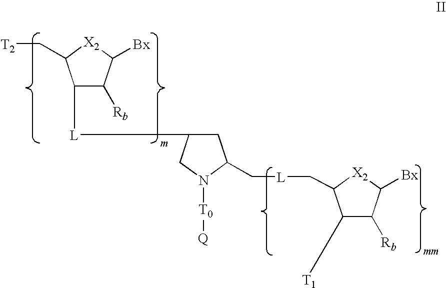
wherein:
T₁ and T₂ are each independently, hydroxyl, a protected hydroxyl or a linkage to a conjugate group;
each L is an internucleoside linking group;
each X₂ is independently, O or S;
each Bₓ is a heterocyclic base moiety;
each R_{b} is independently, H, OH or a 2′-sugar substituent group;
T₀ is a bivalent tethering moiety; and
Q is a conjugate group
m is 0 or from 1 to about 80;
mm is 0 or from 1 to about 80 and
wherein the sum of m plus mm is from 1 to about 80.

21. The oligomeric compound of claim 20 wherein m is 0.

22. The oligomeric compound of claim 20 wherein mm is 0.

23. The oligomeric compound of claim 20 wherein m is at least 1 and mm is at least 1.

24. The oligomeric compound of claim 20 wherein each L is independently, a phosphodiester or phosphorothioate internucleoside linking group.

25. A composition comprising first and second chemically synthesized oligomeric compounds wherein:
at least a portion of said first oligomeric compound is capable of hybridizing with at least a portion of said second oligomeric compound;
at least a portion of said first oligomeric compound is complementary to and capable of hybridizing to a selected nucleic acid target;
wherein at least one of said first and second oligomeric compounds is an oligomeric compound of claim 20; and
said first and said second oligomeric compounds optionally further comprise one or more overhangs, phosphate moieties or capping groups.

26. The composition of claim 25 wherein said first and said second oligomeric compounds comprise a siRNA duplex.

27. The oligomeric compound of claims 20 wherein Q is a lipophilic moiety, vitamin, polymer, peptide, protein, nucleic acid, small molecule, oligosaccharide, carbohydrate cluster, intercalator, minor groove binder, cleaving agent, or cross-linking agent.

28. The oligomeric compound of claim 20 wherein Q is a steroid.

29. The oligomeric compound of claim 20 wherein Q is cholesterol or a cholesterol derivative.

30. The oligomeric compound of claim 20 wherein Q binds to low-density lipoprotein.

31. The oligomeric compound of claim 20 wherein Q is folate or folate derivative.

32. The oligomeric compound of claim 20 comprising a water-soluble polymer.

33. The oligomeric compound of claim 20 wherein Q comprises polyethylene glycol or copolymer thereof.

34. The oligomeric compound of claim 20 wherein said polyentylene glycol or copolymer thereof has a molecular weight of about 20,000 daltons.

35. The oligomeric compound of claim 20 wherein Q comprises a fusogenic peptide or delivery peptide.

36. The oligomeric compound of claim 20 wherein Q comprises a drug.

37. The oligomeric compound of claim 20 wherein Q binds to human serum albumin.

38. The oligomeric compound of claim 20 wherein Q comprises a reporter group.

39. A method of inhibiting gene expression comprising contacting one or more cells, a tissue or an animal with an oligomeric compound of claim 20.

40. A method of inhibiting gene expression comprising contacting one or more cells, a tissue or an animal with a composition of claim 25.

41. The composition of claim 25 wherein said second oligomeric compound is the oligomeric compound of claim 20.
